(19) United States
(12) Patent Application Publication
(10) Pub. No.: US 2023/0095912 A1
(43) Pub. Date: Mar. 30, 2023

(54) Composition and Methods for Selective Degradation of Engineered Proteins

(71) Applicant: Celgene Corporation, Summit, NJ (US)

(72) Inventors: Christopher Walton Carroll, La Jolla, CA (US); Laura Akullian D'Agostino, Sudbury, MA (US); Haibo Liu, Lexington, MA (US); Veerabahu Shanmugasundaram, East Lyme, CT (US); Brook Barajas, Seattle, WA (US)

(73) Assignee: Celgene Corporation, Summit, NJ (US)

(21) Appl. No.: 17/817,777

(22) Filed: Aug. 5, 2022

Related U.S. Application Data
(60) Provisional application No. 63/230,225, filed on Aug. 6, 2021.

(51) Int. Cl. C07K 14/47; C07K 14/725

(52) U.S. Cl. CPC C07K 14/4702; C07K 14/7051; C07K 2319/03; C07K 2319/95; C07K 2319/02

(57) ABSTRACT

The present disclosure relates to engineered polypeptides comprising degradation domains, compounds, compositions, and methods for their preparation and use as for degrading engineered proteins in cells.

### 1. CROSS-REFERENCE TO RELATED APPLICATIONS

This application claims the benefit of priority of U.S. Provisional Application No. 63/230,225, filed Aug. 6, 2021, which is incorporated by reference here in its entirety for any purpose.

### 2. FIELD

The present disclosure relates to engineered polypeptides comprising degradation domains, compounds, compositions, and methods for their preparation and use as for degrading engineered proteins in cells.

### 3. BACKGROUND

Engineered cells comprising an engineered, heterologous polypeptide, such as chimeric antigen receptor T (CAR-T) cells, have been developed for therapeutic use. Modulation of the expression levels of such engineered, heterologous polypeptides may improve the therapeutic benefit of the engineered cells by, for example, decreasing side effects and/or increasing efficacy of the engineered cells.

Accordingly, in one aspect, provided herein are engineered polypeptides and degradation agents, wherein the engineered polypeptides comprise a degradation domain that mediates ubiquitination in cell when the degradation domain binds to a degradation agent.

### 4. SUMMARY

Described herein, in certain embodiments, are compounds and compositions thereof for modulating levels of a heterologous polypeptide in a cell. In various embodiments, the compounds and compositions thereof may be used to decrease the level of the heterologous polypeptide in the cell.

The present embodiments can be understood more fully by reference to the detailed description and examples, which are intended to exemplify non-limiting embodiments.

### 5. BRIEF DESCRIPTION OF THE DRAWINGS

FIG. 1A shows a sequence alignment of human IKZF family proteins IKZF1-IKZF5 (SEQ ID NOS: 48-52). FIG. 1B shows a table of putative G-motif-containing zinc finger sequences of human IKZF1-IKZF5. G-motif sequences are underlined.

FIG. 2 is a diagram of a chimeric antigen receptor (CAR) comprising a C-terminal IKZF1 ZNF2 degron.

FIG. 3 is a schematic of a Jurkat cell reporter-based model system for studying CAR activity and degradation.

FIG. 4A-4C shows that IKZF1 ZNF2-tagged CARs retain function (FIG. 4A) but are only partially degraded by high concentrations of Compound A (FIG. 4C). The structure of Compound A is shown in FIG. 4B.

FIG. 5A shows a schematic of CARs comprising C-terminal IKZF1 ZNF1, 2, and/or 3 degrons. Each of the CARs also comprises an N-terminal CD19-binding scFv. FIG. 5B shows the CARs with C-terminal IKZF1 degrons retain activity. FIG. 5C shows degradation of the CARs with C-terminal IKZF1 degrons in the presence of increasing concentrations of Compound A. FIG. 5D shows that degradation is specific to the wild-type IKZF1 degron.

FIG. 6A-6C show reduction in CAR levels and inhibition of CAR activity after degradation in a Jurkat reporter system.

FIG. 7 shows endogenous Erk signaling is attenuated by CAR degradation.

FIG. 8 shows alignment of G-motif containing C2H2 zinc finger of certain human IKZF family members (SEQ ID NOS: 21, 32, 27, 38, 40, 29, 47, 31, 23, 20, 26 and 37).

FIG. 9 shows predicted C2H2 zinc finger degrons from various human proteins (SEQ ID NOS: 72-109).

FIG. 10A shows the structure of Compound B. FIG. 10B shows ubiquitination of modified G-motifs from Ikaros ZNF2 using an in vitro ubiquitination assay.

FIG. 11A shows the structure of Compound C. FIG. 11B-11C show that degradation of the IKZF1 ZNF2_3 Q1F degron-tagged CARs is CRBN and ubiquitin-proteasome pathway (UPP) dependent.

FIG. 12A-12B shows IKZF1 ZNF2_3 Q1F-tagged CAR degradation decreases CAR levels and signaling in a Jurkat reporter assay.

FIG. 13A shows the structure of a IKZF1 ZNF2_3 Q1F tagged CAR. FIG. 13B shows that the IKZF1 ZNF2_3 Q1F tagged CAR expression is titratable with Compound C in primary T cells.

FIG. 14A-14D shows IKZF1 ZNF2_3 Q1F tagged CAR function is titratable with Compound C in primary T cells.

FIG. 15 is a schematic of the chronic antigen stimulation assay used to test functional persistence.

FIG. 16A shows the structure of Compound D. FIG. 16B-16C show transiently rested CAR T cells are less activated by chronic antigen exposure and maintain a more naïve-like phenotype.

FIG. 17A-17B shows transiently rested CAR T cells produce more proinflammatory cytokines and demonstrate better anti-tumor activity after chronic antigen exposure than cells without rest.

FIG. 18A-18C shows Q1F degron-tagged CAR can be reversibly downregulated in vivo.

FIG. 19A-19D shows Q1F degron-tagged CAR downregulation decreases tumor responsive expansion in vivo.

FIG. 20A-20D shows in-frame degron-tag knock-in to endogenous AURKA or TOX locus allows compound-mediated control of protein levels.

### 6. DETAILED DESCRIPTION

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated. As used herein, the terms “about” and “approximately” mean±20%, ±10%, ±5%, or ±1% of the indicated range, value, or structure, unless otherwise indicated.

As used herein, an “engineered polypeptide” is a polypeptide having an amino acid sequence that does not occur in nature. While portions of an engineered polypeptide may occur in nature, the engineered polypeptide as a whole does not. In some embodiments, an engineered polypeptide comprises a naturally-occurring amino acid sequence that has been modified, for example, by fusing it to, or inserting into it, a degradation domain. In some such embodiments, the resulting engineered polypeptide substantially retains the activity of the original naturally-occurring polypeptide. In some embodiments, an engineered polypeptide comprises two or more, or three or more, or four or more domains derived from two or more, or three or more, or four or more naturally-occurring polypeptides. In some embodiments, the engineered polypeptide comprises a degradation domain.

As used herein, “degron” and “degradation domain” are used interchangeably and mean an amino acid sequence that, when present in a polypeptide in a cell, results in ubiquitination of the polypeptide by a ubiquitin ligase in the presence of a compound that binds to both the degradation domain and the ubiquitin ligase. In some embodiments, the compound binds to the degradation domain and with cereblon. In some embodiments, an engineered polypeptide comprises a degradation domain. Following ubiquitination by the ubiquitin ligase, the polypeptide that comprises the degradation domain may be degraded.

### Exemplary Engineered Polypeptides

Engineered polypeptides comprising a degradation domain are provided herein. In some embodiments, the engineered polypeptide is a CAR. In some embodiments, such polypeptides comprise a transmembrane domain, extracellular domain, and intracellular domain. In some such embodiments, the degradation domain is located in the intracellular domain of engineered polypeptide. In some embodiments, the extracellular domain comprises a ligand, ligand-binding domain, or an antigen-binding domain. In some embodiments, the antigen-binding domain binds a cancer antigen. In some embodiments, the antigen-binding domain comprises an antibody light or heavy chain variable region, or a scFv. In some embodiments, the antigen-binding domain comprises a single-domain antibody antigen-binding domain. In some embodiments, the intracellular domain comprises at least one co-stimulatory domain. In some embodiments, the intracellular domain comprises at least one signaling domain, such as an ITAM signaling domain. In some embodiments, the engineered polypeptide is a CAR comprising a degradation domain, as further described below.

In some embodiments, an engineered polypeptide is based on a naturally-occurring protein in which a degradation domain has been inserted by genetic engineering or to which a degradation domain has been fused. The resulting engineered polypeptide may comprise additional naturally-occurring or non-naturally-occurring amino acid sequence. In some embodiments, the engineered polypeptide is based on a naturally-occurring nuclear or cytoplasmic protein. In some embodiments, the engineered polypeptide substantially retains the activity of the naturally-occurring protein. Degradation of the engineered polypeptide may be accomplished by contacting a cell that expresses the engineered polypeptide, such as by administering, a degradation agent. In some such embodiments, the degradation domain is derived from an Ikaros Family Zinc Finger (ZNF) amino acid sequence, and the degradation agent is a small molecule that binds to a ubiquitin ligase, such as an E3 ligase. Administration of the degradation agent to cells expressing the engineered polypeptide comprising the degradation domain results in ubiquitination of the engineered polypeptide comprising the degradation domain by the E3 ligase and degradation of the engineered polypeptide. In some embodiments, the degradation agent is a compound that binds cereblon and the degradation domain.

In some embodiments, the engineered polypeptide comprises a naturally-occurring protein and a degradation domain fused to, or inserted within, the naturally-occurring protein. When the degradation domain is “fused to” the protein, the engineered polypeptide may comprise a linker connecting the degradation domain to the protein, such as an amino acid linker. Such amino acid linkers may be any length, and for example, 1-50, 1-40, 1-30, 1-20, 1-10, or 1-5 amino acids. In some embodiments, amino acid linkers are composed of glycine and serine.

Nonlimiting exemplary proteins to which a degradation domain may be fused or into which a degradation domain may be inserted include PRDM1, TGFBR2, CASP8, CBLB, CD5, CISH, CGKA, DGKz, MAP4K1, ARID2, BACH2, CHX37, KLF2, KLF3, KLF6, MAF, SIGLEC9, TOX, ZBTB32, PTPN2, AKT1, PIK3CD, MT1E, MT2A, CSK, ITK, PAG1, PDCD4, ZC3H12A, DNMT1, DNMT3A, PRBM1, STK4, TET2, BNIP3, FAS, CBL, BGAT5, RNF128, STK17B, TRIB1, TXNIP, UBASH3A, BATF, FLI1, IKZF1, IKZF2, IRF4, NFATC1, NR4A1, MAP2K1, MAP2K2, MAP4K4, PPARGC1A, RELB, TMEM173, USP10, MT1A, PP2A family members, RASA2, NR4A2, NR4A3, AHR, CD70, LHALS1, SOCS1, SOCS2, SOCS3, TAZ, USP21, or YAP1. In some embodiments, the protein is a mammalian protein, such as a human protein.

In some embodiments, a degradation domain is fused to, or inserted into, an endogenous protein in a cell. In some such embodiments, a sequence encoding the degradation domain may be inserted into the genome of a cell that expresses the endogenous protein such that the engineered polypeptide is expressed, comprising the degradation domain fused to or inserted into the endogenous protein. Various methods of inserting a nucleic acid sequence, such as a sequence encoding a degradation domain, into the genome of a cell are known in the art, including, for example, Adeno-associated Virus (AAV)-mediated or non-viral homology-directed recombination via CRISPR/Cas, lentiviral transduction, or transposon delivery. In some embodiments, a nucleic acid sequence encoding a degradation domain is fused to or inserted into an endogenous protein in an immune cell, such as a T lymphocyte. In some such embodiments, T cells are isolated, engineered to express the engineered polypeptide, and administered to a patient. Following administration to the patient, a degradation agent may be subsequently administered when degradation of the engineered polypeptide is desired.

In some embodiments, a nucleic acid sequence encoding an engineered polypeptide is introduced into a cell. Methods of introducing nucleic acids into cells are known in the art, and include synthetic vectors, lentiviral or retroviral vectors, autonomously replicating plasmids, a virus {e.g., a retrovirus, lentivirus, adenovirus, or herpes virus), or the like, containing nucleic acid (polynucleotides) encoding the engineered polypeptides described herein.

Exemplary Degradation Domains

The engineered polypeptides provided herein comprise a degradation domain. In some embodiments, the degradation domain comprises an amino acid sequence that binds to a degradation agent. The degradation agent associates with the degradation domain and with a ubiquitin ligase, resulting in ubiquitination of the engineered polypeptide.

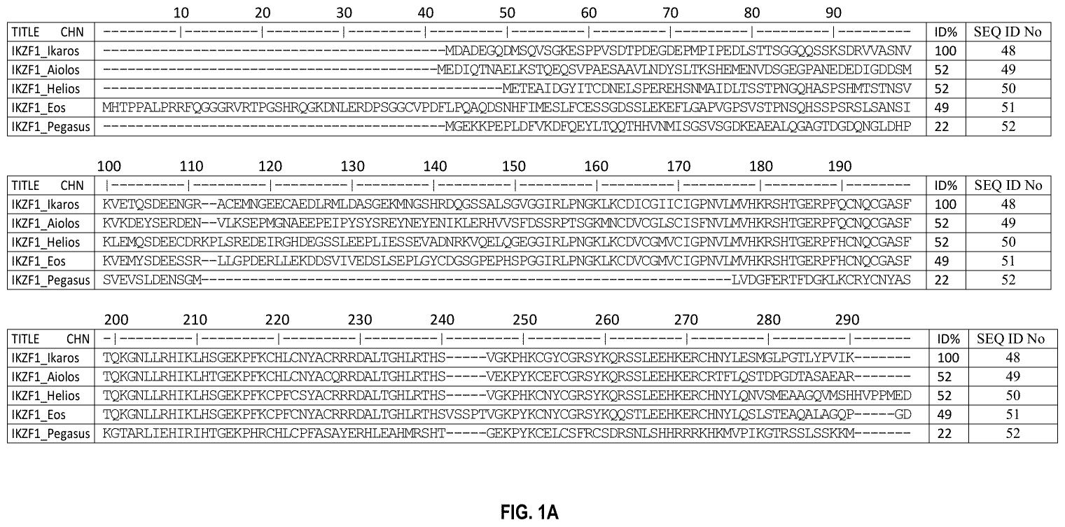

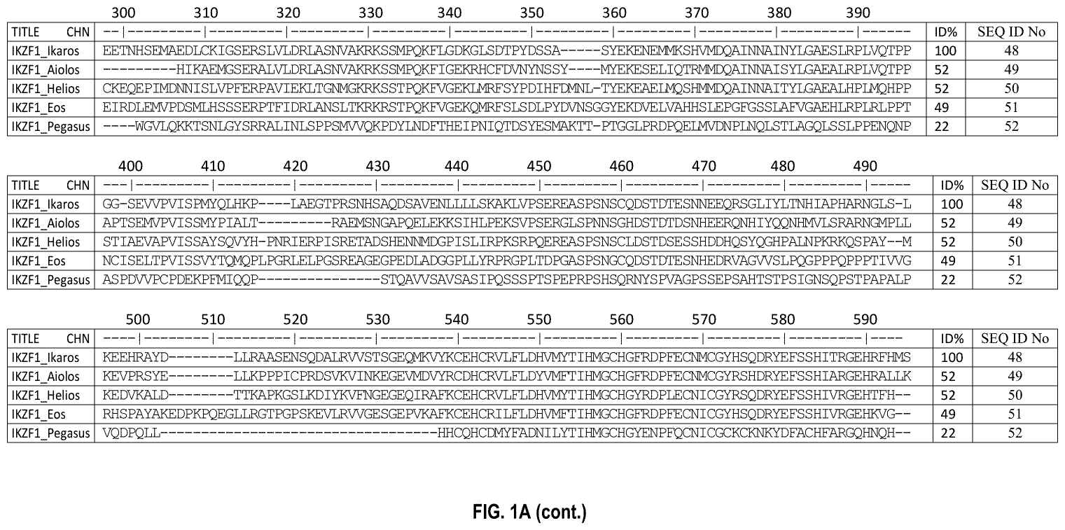

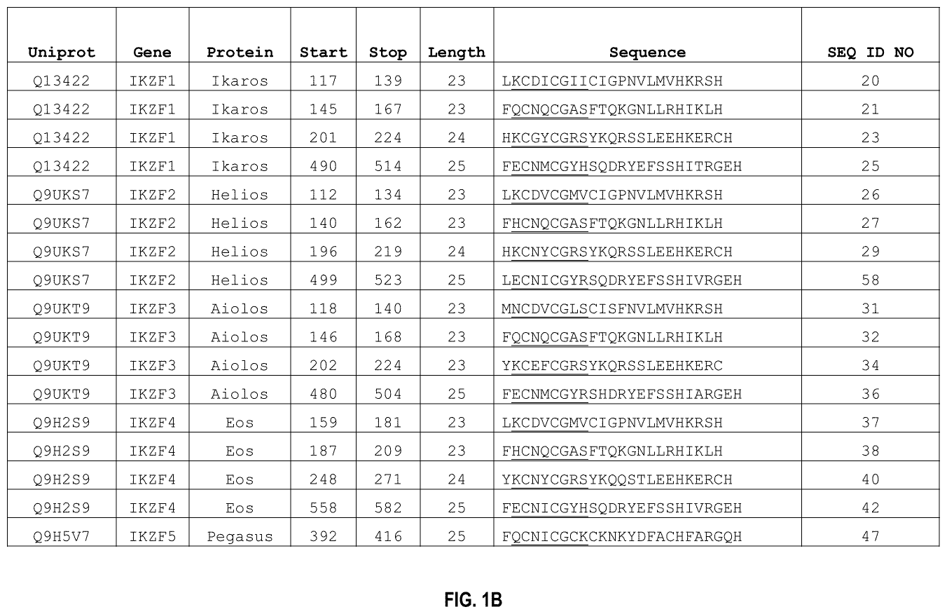

In some embodiments, the degradation domain comprises an amino acid sequence derived from a G-motif of an Ikaros family protein, such as Ikaros, Helios, Aiolos, Eos or Pegasus. Nonlimiting exemplary G-motifs are underlined in the sequences shown in FIG. 1B. A degradation domain provided herein, in some embodiments, comprises an amino acid sequence that is modified from a native G-motif sequence by replacing the amino acid in the first position with a phenylalanine (F). In some embodiments, the degradation domain is derived from a G-motif that naturally comprises Q in the first position, so the degradation domain comprises a Q1F substitution.

In some embodiments, the degradation domain comprises the amino acid sequence FCX₁X₂CGX₃X₄ (SEQ ID NO: 1). In some embodiments, X₁ is selected from asparagine, aspartate, glycine, glutamine, methionine, histidine, tryptophan, isoleucine, arginine, leucine, valine, threonine, and phenylalanine; X₂ is selected from glutamine, arginine, histidine, leucine, phenylalanine, tyrosine, tryptophan, isoleucine, valine, and methionine; X₃ is selected from alanine, serine, cysteine, arginine, leucine, isoleucine, methionine, and glycine; and X₄ is selected from serine, methionine, lysine, isoleucine, valine, histidine, glutamine, arginine, phenylalanine, and tryptophan. In some embodiments, X₁ is selected from asparagine, glutamine, methionine, histidine, tryptophan, isoleucine, arginine, leucine, valine, threonine, and phenylalanine. In some embodiments, X₂ is selected from glutamine, arginine, histidine, leucine, phenylalanine, tyrosine, tryptophan, isoleucine, and methionine. In some embodiments, X₃ is selected from alanine, serine, cysteine, and glycine. In some embodiments, X₄ is selected from serine, methionine, histidine, glutamine, arginine, phenylalanine, and tryptophan. In some embodiments, X₁ is asparagine. In some embodiments, X₂ is glutamine. In some embodiments, X₃ is alanine or serine. In some embodiments, X₃ is alanine. In some embodiments, X₄ is serine. In some embodiments, the degradation domain of the engineered polypeptide comprises the amino acid sequence FCNQCGAS (SEQ ID NO: 3).

In some embodiments, the degradation domain comprises the amino acid sequence FCX₁X₂CGX₃X₄X₅ (SEQ ID NO: 2), wherein X₁, X₂, X₃, and X₄ are as defined above. In some embodiments, X₅ is selected from phenylalanine, tryptophan, methionine, arginine, histidine, leucine, tyrosine, cysteine, and glutamine. In some embodiments, X₅ is selected from phenylalanine, tryptophan, methionine, arginine, histidine, leucine, tyrosine, and glutamine. In some embodiments, X₅ is selected from phenylalanine, tryptophan, methionine, leucine, tyrosine, and glutamine. In some embodiments, X₅ is phenylalanine.

In various embodiments, the degradation domain comprises at least one zinc finger domain that comprises the modified G-motif discussed above. In some embodiments, at least one zinc finger domain is derived from an Ikaros family protein, such as Ikaros, Helios, Aiolos, Eos or Pegasus. Nonlimiting exemplary zinc fingers comprising G-motifs are shown in FIG. 1B. In various embodiments, the degradation domain comprises one, two, three, or four zinc finger domains. In some embodiments, the degradation domain comprises one zinc finger domain that comprises a G-motif and at least one zinc finger domain that does not comprise a G-motif. The zinc finger domains may or may not be derived from the same protein. In some embodiments, the degradation domain comprises two zinc finger domains.

In some embodiments, the degradation domain comprises an amino acid sequence that is at least 85%, at least 90%, or at least 95% identical to zinc finger 2 (ZNF2) of human Ikaros. In some embodiments, the degradation domain comprises an amino acid sequence that is at least 85%, at least 90%, or at least 95% identical to zinc finger 2 (ZNF2) of human Ikaros, and at least one additional zinc finger domain, such as at least one additional zinc finger domain of an Ikaros family protein. In some embodiments, the degradation domain comprises an amino acid sequence that is at least 85%, at least 90%, or at least 95% identical to zinc finger 2 (ZNF2) of human Ikaros and ZNF1 or ZNF3 of human Ikaros. In some embodiments, the degradation domain comprises an amino acid sequence that is at least 85%, at least 90%, or at least 95% identical to zinc finger 2 (ZNF2) and zinc finger 3 (ZNF3) of human Ikaros.

In some embodiments, the degradation domain comprises an amino acid sequence that is at least 85%, at least 90%, or at least 95% identical to amino acids 145-167 of human Ikaros (FQCNQCGASFTQKGNLLRHIKLH; SEQ ID NO: 21). In some embodiments, the degradation domain comprises an amino acid sequence that is at least 85%, at least 90%, or at least 95% identical to amino acids 140-162 of human Helios (FHCNQCGASFTQKGNLLRHIKLH; SEQ ID NO: 27). In some embodiments, the degradation domain comprises an amino acid sequence that is at least 85%, at least 90%, or at least 95% identical to amino acids 146-168 of human Aiolos (FQCNQCGASFTQKGNLLRHIKLH; SEQ ID NO: 32). In some embodiments, the degradation domain comprises an amino acid sequence that is at least 85%, at least 90%, or at least 95% identical to amino acids 187-209 of human Eos (FHCNQCGASFTQKGNLLRHIKLH; SEQ ID NO: 38).

In some embodiments, the degradation domain comprises an amino acid sequence that is at least 85%, at least 90%, or at least 95% identical to amino acids 141-168 of human Ikaros (GERPFQCNQCGASFTQKGNLLRHIKLHS; SEQ ID NO: 15). In some embodiments, the degradation domain comprises an amino acid sequence that is at least 85%, at least 90%, or at least 95% identical to amino acids 136-163 of human Helios (GERPFHCNQCGASFTQKGNLLRHIKLHS; SEQ ID NO: 60). In some embodiments, the degradation domain comprises an amino acid sequence that is at least 85%, at least 90%, or at least 95% identical to amino acids 142-169 of human Aiolos (GERPFQCNQCGASFTQKGNLLRHIKLHT; SEQ ID NO: 61). In some embodiments, the degradation domain comprises an amino acid sequence that is at least 85%, at least 90%, or at least 95% identical to amino acids 183-210 of human Eos (GERPFHCNQCGASFTQKGNLLRHIKLHS; SEQ ID NO: 62).

In some embodiments, the degradation domain comprises an amino acid sequence that is at least 85%, at least 90%, or at least 95% identical to amino acids 141-196 of human Ikaros (GERPFQCNQC GASFTQKGNL LRHIKLHSGE KPFKCHLCNY ACRRRDALTG HLRTHS; SEQ ID NO: 6). In some embodiments, the degradation domain comprises an amino acid sequence that is at least 85%, at least 90%, or at least 95% identical to amino acids 136-191 of human Helios (GERPFHCNQC GASFTQKGNL LRHIKLHSGE KPFKCPFCSY ACRRRDALTG HLRTHS; SEQ ID NO: 63). In some embodiments, the degradation domain comprises an amino acid sequence that is at least 85%, at least 90%, or at least 95% identical to amino acids 142-197 of human Aiolos (GERPFQCNQC GASFTQKGNL LRHIKLHTGE KPFKCHLCNY ACQRRDALTG HLRTHS; SEQ ID NO: 64). In some embodiments, the degradation domain comprises an amino acid sequence that is at least 85%, at least 90%, or at least 95% identical to amino acids 183-238 of human Eos (GERPFHCNQC GASFTQKGNL LRHIKLHSGE KPFKCPFCNY ACRRRDALTG HLRTHS; SEQ ID NO: 65).

In some embodiments, the degradation domain comprises the amino acid sequence: GERPFFCX₁X₂CGX₃X₄X₅TQKGNLLRHIKLHSGEKPFKCHLCNYACRRRDALTGHLRTHS (SEQ ID NO: 5), wherein X₁, X₂, X₃, and X₄, and X₅ are as defined above. In some embodiments, the degradation domain comprises the amino acid sequence: GERPFFCX₁X₂CGX₃X₄X₅TQKGNLLRHIKLHSGEKPFKCPFCSYACRRRDALTGHLRTHS (SEQ ID NO: 66), wherein X₁, X₂, X₃, and X₄, and X₅ are as defined above. In some embodiments, the degradation domain comprises the amino acid sequence: GERPFFCX₁X₂CGX₃X₄X₅TQKGNLLRHIKLHTGEKPFKCHLCNYACQRRDALTGHLRTH S (SEQ ID NO: 67), wherein X₁, X₂, X₃, and X₄, and X₅ are as defined above. In some embodiments, the degradation domain comprises the amino acid sequence: GERPFFCX₁X₂CGX₃X₄X₅TQKGNLLRHIKLHSGEKPFKCPFCNYACRRRDALTGHLRTHS (SEQ ID NO: 68), wherein X₁, X₂, X₃, and X₄, and X₅ are as defined above.

In some embodiments, the degradation domain comprises the amino acid sequence: GERPFFCNQCGASFTQKGNLLRHIKLHSGEKPFKCHLCNYACRRRDALTGHLRTHS (SEQ ID NO: 7). In some embodiments, the degradation domain comprises the amino acid sequence: GERPFFCNQCGASFTQKGNLLRHIKLHSGEKPFKCPFC SYACRRRDALTGHLRTHS (SEQ ID NO: 69). In some embodiments, the degradation domain comprises the amino acid sequence: GERPFFCNQCGASFTQKGNLLRHIKLHTGEKPFKCHLCNYACQRRDALTGHLRTHS (SEQ ID NO: 70). In some embodiments, the degradation domain comprises the amino acid sequence:

(SEQ ID NO: 71)
GERPFFCNQCGASFTQKGNLLRHIKLHSGEKPFKCPFCNYACRRRDALTG
HLRTHS.

Exemplary Chimeric Antigen Receptor Constructs

The ability to modulate the expression of CARs by degrading them in the presence of a degradation agent has many advantages over the lack of an ability to modulate CAR expression. For example, on-target but off-tumor effects mediated by therapeutic immune cells expressing CARs, which potentially lead to toxicity, can be reduced or eliminated by degrading the CAR. A CAR-mediated immune response that is too strong can be reduced or eliminated by degrading the CAR. T cell dysfunction caused by chronic activation and overexpression of checkpoints can be avoided by cycling the expression of the CAR and/or titrating expression of the CAR. Such CAR degradation is accomplished herein by expressing a CAR that comprises a degradation domain provided herein and administering a degradation agent, as needed. In some such embodiments, the degradation agent is a small molecule that binds to a ubiquitin ligase, such as an E3 ligase. Administration of the degradation agent to cells expressing the CAR polypeptide comprising the degradation domain results in ubiquitination of the CAR polypeptide comprising the degradation domain by the E3 ligase and degradation of the CAR polypeptide. In some embodiments, the degradation agent is a compound that binds cereblon and the degradation domain.

Provided herein are engineered polypeptides comprising or consisting of Chimeric Antigen Receptors (CARs) comprising (a) components of a CAR, such as an antigen-binding domain, a transmembrane domain, a cell signaling domain, and/or a co-stimulatory domain, and (b) a degradation domain. When the CAR fused to the degradation domain is expressed in an immune cell (e.g., in a T lymphocyte or natural killer cell) in the presence of a degradation agent, such as a cereblon-binding compound, an E3 ligase, such as cereblon, and the degradation domain in the CAR bind the degradation agent, resulting in formation of an E3 ligase complex that ubiquitinates the degradation domain. Thus, activity of the CARs described herein (e.g., in vivo activity) can be controlled by contacting a cell expressing the CAR comprising the degradation domain (e.g., T lymphocytes engineered to express said CAR polypeptides) with a degradation agent, such as a cereblon-binding compound.

In some embodiments, provided herein is an engineered polypeptide that is a CAR comprising an antigen-binding domain, a transmembrane domain, an intracellular, primary signaling domain, and a degradation domain. In some embodiments, the degradation domain comprises an amino acid sequence provided herein.

In some embodiments, the engineered polypeptide is a CAR comprising, in order from amino-terminus to carboxy-terminus, an antigen-binding domain, a transmembrane domain, a primary T cell signaling domain, and/or a co-stimulatory domain, and a degradation domain. In some embodiments, the degradation domain is located at the C-terminus of the CAR. In some embodiments, the degradation domain comprises an amino acid sequence provided herein. In some embodiments, the CAR comprises a co-stimulatory domain.

In some embodiments, the engineered polypeptide is a CAR comprising, in order from amino-terminus to carboxy-terminus, (i) an extracellular domain [ECD]—a transmembrane domain [TM]—a co-stimulatory domain [CoD]—a signaling domain [SigD]—a degradation domain [DD]. In some embodiments, the engineered polypeptide is a CAR comprising, in order from amino-terminus to carboxy-terminus, ECD-TM-CoD-DD-SigD. In some embodiments, the engineered polypeptide is a CAR comprising, in order from amino-terminus to carboxy-terminus, ECD-TM-DD-CoD-SigD. Degradation domains may also be inserted within another domain, such as within the co-stimulatory domain or within the signaling domain, preferably such that the desired activity of the domains is retained.

Exemplary Antigen Binding Domains

The antigen binding domains of the CARs provided herein can be any polypeptide domain, motif or sequence that binds to an antigen.

In certain embodiments, the antigen binding domain of the CARs described herein is an antigen binding portion of a receptor. In some embodiments, the antigen binding domain of the CARs described herein is a receptor for a ligand produced by a tumor cell.

In certain embodiments, the antigen binding domain of the CARs described herein is an antigen-binding portion of an antibody. In some embodiments, the antigen binding domain of the CARs described herein is an antibody, an antibody chain, a single chain antibody, or an antigen binding portion thereof, an Fc domain, a glycophosphatidylinositol anchor domain, or scFv antibody fragment.

In certain embodiments, the antigen binding domain of the CARs described herein is a peptide-based macromolecular antigen binding agent, e.g., a phage display protein.

In certain embodiments, antigen binding by an antigen binding domain of a CAR described herein is restricted to antigen presentation in association with major histocompatibility complexes (MEW). In certain embodiments, antigen binding by an antigen binding domain of a CAR described herein is MHC-unrestricted.

The antigen bound/recognized by the antigen binding domain of the CARs described herein can be any antigen of interest. In some embodiments, the antigen is an antigen that is expressed on the surface of a cell (e.g., a tumor cell, such as a solid tumor cell or a blood cancer tumor cell).

In some embodiments, the antigen bound/recognized by the antigen binding domain of the CARs described herein is an antigen on a tumor cell, for example, the antigen is a tumor-specific antigen (TSA) or a tumor-associated antigen (TAA). Exemplary tumor cell antigens that can be recognized by the CARs described herein (i.e., bound by the antigen-binding domain of the CARs) include, without limitation, 4-1BB, 5T4, 8H9, B7-H6, adenocarcinoma antigen, a-fetoprotein, B Cell Maturation Antigen (BCMA), BAFFR, B-lymphoma cell, C242 antigen, CA9, carcinoembryonic antigen, CA-125, carbonic anhydrase 9 (CA-IX), CCR4, CD3, CD4, CD19, CD20, CD22, CD23 (IgE receptor), CD28, CD30 (T FRSF8), CD33, CD38, CD40, CD44v6, CD44v7/8, CD51, CD52, CD56, CD70 CD74, CD80, CD123, CD152, CD171, CD200, CD221, CE7, CEA, C-MET, CLAUDIN6, CLAUDIN18.3, CNT0888, CTLA-4, DRS, EpCAM, ErbB2, ErbB3/4, EGFR, EGFRγIII, EphA2, EGP2, EGP40, FAP, Fetal AchR, fibronectin extra domain-B, folate receptor-a, folate receptor 1, G250/CAIX, GD2, GD3, glycoprotein 75, GP MB, HER2/neu, HGF, HLA-AI MAGE A1, HLA-A2 NY-ESO-1, HMW-MAA, human scatter factor receptor kinase, IGF-1 receptor, IGF-I, IgG1, IL-6, IL-13, IL-13 receptor a2, IL-11 receptor a, insulin-like growth factor I receptor, integrin a5I31, integrin avI33, Kappa light chain, L1-CAM, Lambda light chain, Lewis Y, mesothelin, MORAb-009, MS4A1, MUC1, MUC1 6, mucin CanAg, NCAM, N-glycolylneuraminic acid, NKG2D ligands, NPC-IC, PDGF-R a, PDL192, phosphatidylserine, prostate-specific cancer antigen (PSCA), prostatic carcinoma cells, PSMA, PSC1, RANKL, RON, ROR1, SCH 900105, SDC1, SLAMF7, sp17, TAG72, tenascin C, TGF (32, TGF-β, TL1A, TRAIL-R1, TRAIL-R2, tumor antigen CTAA16.88, UPK1B, VEGF-A, VEGF receptors, VEGFR-1, VEGFR2, TEM1, TEM8, and/or vimentin.

In some embodiments, the antigen bound/recognized by the antigen binding domain of the CARs described herein is an antigen expressed on or associated with a tumor cell of a lymphoma/leukemia, a lung cancer, a breast cancer, a prostate cancer, an adrenocortical carcinoma, a thyroid carcinoma, a nasopharyngeal carcinoma, a melanoma, e.g., a malignant melanoma, a skin carcinoma, a colorectal carcinoma, a desmoid tumor, a desmoplastic small round cell tumor, an endocrine tumor, an Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipoma, or the like.

In some embodiments, the antigen bound/recognized by the antigen binding domain of the CARs described herein is an antigen expressed on or associated with a tumor cell of chronic lymphocytic leukemia (small lymphocytic lymphoma), B-cell prolymphocytic leukemia, lymphoplasmacytic lymphoma, Waldenstrom macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, or a non-Hodgkin lymphoma.

In some embodiments, the antigen bound/recognized by the antigen binding domain of the CARs described herein is a non-tumor-associated antigen or a non-tumor-specific antigen. In certain embodiments, the antigen is related to an aspect of a tumor, e.g., the tumor environment. For example, a tumor can induce an inflammatory state in tissue surrounding the tumor, and can release angiogenic growth factors, interleukins, and/or cytokines that promote angiogenesis into and at the periphery of the tumor. Thus, in certain embodiments, the antigen is a growth factor, a cytokine, or an interleukin (e.g., a growth factor, cytokine, or interleukin associated with angiogenesis or vasculogenesis). Such growth factors, cytokines, and interleukins can include, without limitation, vascular endothelial growth factor (VEGF), basic fibroblast growth factor (bFGF), platelet-derived growth factor (PDGF), hepatocyte growth factor (HGF), insulin-like growth factor (IGF), and interleukin-8 (IL-8).

In some embodiments, the antigen bound/recognized by the antigen binding domain of the CARs described herein is a damage associated molecular pattern molecule (DAMP; also known as an alarmin) released by normal tissue in response to localize damage caused by a tumor. Exemplary DAMPs to which the antigen-binding domain of the CARs described herein can bind include, without limitation, heat shock protein, chromatin-associated protein high mobility group box 1 (HMGB1), S100A8 (MRP8, calgranulin A), S100A9 (MRP 14, calgranulin B), serum amyloid A (SAA), deoxyribonucleic acid, adenosine triphosphate, uric acid, and heparin sulfate.

Exemplary Transmembrane Domains

As used herein, “transmembrane domain” includes pass-through transmembrane domains in which the polypeptide comprising the transmembrane domain comprises both intracellular and extracellular domains, and membrane-anchoring domains in which the polypeptide comprising the transmembrane domain comprises an intracellular domain but no extracellular domain.

The transmembrane domains of the engineered polypeptides described herein can comprise any molecule known in the art to function as a transmembrane domain, e.g., known by one of skill in the art to function in the context for which it will be used, such as in a CAR. The transmembrane domains engineered polypeptides described herein can be obtained or derived from the transmembrane domain of any transmembrane protein, and can include all or a portion of such transmembrane domain.

In some embodiments, the transmembrane domain of an engineered polypeptide described herein, such as a CAR, is obtained or derived from a T-cell receptor, e.g., the transmembrane domain of the engineered polypeptide described herein is obtained or derived from the alpha chain of a T-cell receptor, the beta chain of a T-cell receptor, the zeta chain of a T-cell receptor.

In some embodiments, the transmembrane domain of the engineered polypeptide described herein is obtained or derived from CD28, CD3s, CD45, CD4, CD5, CD8, CD9, CD16, CD22, CD33, CD37, CD64, CD80, CD86, CD134, CD137, CD154, ICOS, TIM3, LAB3, TIGIT, PD1, or CTLA4, a cytokine receptor, an interleukin receptor, or a growth factor receptor.

Exemplary Signaling Domains

The primary cell signaling domain of the CARs described herein can comprise any molecule known in the art to function as a cell signaling domain, e.g., known by one of skill in the art to function in the CAR context. In some embodiments, the cell signaling domain of the CARs described herein comprises a primary T cell signaling domain.

In some embodiments, the primary cell signaling domain of the CARs described herein is or comprises ZAP-70, or a signal-transducing variant thereof.

In some embodiments, the primary cell signaling domain of the CARs described herein is or comprises an IT AM. In some embodiments, said IT AM is the IT AM of CD3ε, CD3ζ, CD3η, FcRγ, FcRβ, CD3δ, CD3γ, CD5, CD22, CD20, CD79a, CD79b, CD278 (ICOS), FcERI, CD66d, DAP10, or DAP12.

Exemplary Co-Stimulatory Domains

In certain embodiments, the CARs described herein comprise a co-stimulatory domain. The co-stimulatory domain(s) of the CARs described herein can comprise any molecule known in the art to function as a co-stimulatory domain, e.g., known by one of skill in the art to function in the CAR context.

In some embodiments, the co-stimulatory domain of a CAR described herein is obtained or derived from a co-stimulatory CD27 polypeptide sequence, a co-stimulatory CD28 polypeptide sequence, a co-stimulatory OX40 (CD134) polypeptide sequence, a co-stimulatory 4-1BB (CD137) polypeptide sequence, or a co-stimulatory inducible T-cell co-stimulatory (ICOS) polypeptide sequence.

In some embodiments, the co-stimulatory domain of the CARs described herein is or comprises 4-1BB (CD137), CD28, OX40, an activating K cell receptor, BTLA, a Toll ligand receptor, CD2, CD7, CD27, CD30, CD40, CDS, ICAM-L LFA-1 (CD1 1a/CD18), B7-H3, CDS, ICAM-1, ICOS (CD278), RANK, GITR, BAFFR, LIGHT, HVEM (LIGHTR), KIRDS2, SLAMF7, Kp80 (KLRF1), Kp44, Kp30, Kp46, CD 19, CD4, CD8a, CD8p, IL2Rp, IL2Ry, IL7Ra, ITGA4, VLA1, CD49a, ITGA4, IA4, CD49D, ITGA6, VLA-6, CD49f, ITGAD, CD1 1d, ITGAE, CD103, ITGAL, CD1 1a, LFA-1, ITGAM, CD1 1b, ITGAX, CD1 1c, ITGB 1, CD29, ITGB2, IL15Ra, IL7R, CD18, CD132, LFA-1, ITGB7, KG2D, KG2C, T FR2, TRANCE/RA KL, DNAM1 (CD226), SLAMF4 (CD244, 2B4), CD84, CD96 (Tactile), CEACAM1, CRT AM, Ly9 (CD229), CD 160 (BY55), PSGL1, CD 100 (SEMA4D), CD69, SLAMF6 (NTB-A, Ly108), SLAM (SLAMF1, CD 150, IPO-3), BLAME (SLAMF8), SELPLG (CD 162), LTBR, LAT, GADS, SLP-76, PAG/Cbp, CD19a, DAP10, DAP 12, a ligand of CD83, an MHC class I molecule, a TNF receptor protein, an immunoglobulin-like protein, a cytokine receptor, an integrin, and/or a signaling lymphocytic activation molecule.

Other Exemplary Components

In certain embodiments, the engineered polypeptides, such as CARs, described herein further comprise a T cell survival motif. The T cell survival motif can be any amino acid sequence or motif that facilitates the survival of a T lymphocyte after stimulation by an antigen. In certain embodiments, the T cell survival motif is, or is derived from, CD3, CD28, an intracellular signaling domain of IL-7 receptor (IL-7R), an intracellular signaling domain of IL-12 receptor, an intracellular signaling domain of IL-15 receptor, an intracellular signaling domain of IL-21 receptor, or an intracellular signaling domain of transforming growth factor 0 (TGFB) receptor.

Exemplary Modifications

In certain embodiments, the engineered polypeptides provided herein are modified by, e.g., acylation, amidation, glycosylation, methylation, phosphorylation, sulfation, sumoylation, and/or ubiquitylation (or other protein modifications).

In certain embodiments, the engineered polypeptides provided herein are labeled with a label capable of providing a detectable signal, e.g., a radioisotope or fluorescent compound.

In certain embodiments, one or more side chains of the engineered provided herein are derivatized, e.g., derivatization of lysinyl and amino terminal residues with succinic or other carboxylic acid anhydrides, or derivatization with, e.g., imidoesters such as methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate. In certain embodiments, carboxyl side groups, aspartyl or glutamyl, may be selectively modified by reaction with carbodiimides (R—N═C═N—) such as 1-cyclohexyl-3-(2-morpholinyl-(4-ethyl)carbodiimide or 1-ethyl-3-(4-azonia-4,4-dimethylpentyl)carbodiimide.

Exemplary Nucleic Acids

Provided herein are nucleic acids encoding the engineered polypeptides described herein. Nucleic acids useful in the production of the engineered polypeptides described herein include DNA, RNA, and nucleic acid analogs. Nucleic acid analogs can be modified at the base moiety, sugar moiety, or phosphate backbone, and can include deoxyuridine substitution for deoxythymidine, 5-methyl-2′-deoxycytidine or 5-bromo-2′-deoxycytidine substitution for deoxycytidine. Modifications of the sugar moiety can include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, for example, Summerton and Weller (1997) Antisense Nucleic Acid Drug Dev. 7: 187-195; and Hyrup et al. (1996) Bioorgan. Med. Chain. 4:5-23. In addition, the deoxyphosphate backbone can be replaced with, for example, a phosphorothioate or phosphorodithioate backbone, a phosphoroamidite, or an alkyl phosphotriester backbone.

In certain embodiments, the engineered polypeptides-encoding nucleic acids described herein are comprised within a nucleic acid vector. For example, cells of interest, e.g., T lymphocytes, can be transformed using synthetic vectors, lentiviral or retroviral vectors, autonomously replicating plasmids, a virus {e.g., a retrovirus, lentivirus, adenovirus, or herpes virus), or the like, containing nucleic acid (polynucleotides) encoding the engineered polypeptides described herein. In some embodiments, the vector comprising the engineered polypeptides described herein is a retroviral vector. In some embodiments, the vector comprising the nucleic acid encoding the engineered polypeptides described herein is a lentiviral vector. Lentiviral vectors suitable for transformation of cells, e.g., T lymphocytes, include, but are not limited to the lentiviral vectors described in U.S. Pat. Nos. 5,994,136; 6,165,782; 6,428,953; 7,083,981; and 7,250,299. HIV vectors suitable for transformation of cells, e.g., T lymphocytes, include, but are not limited to the vectors described in U.S. Pat. No. 5,665,577.

In certain embodiments, the engineered polypeptides-encoding nucleic acids described herein are operably linked to a promoter. In some embodiments, said promoter is a T cell-specific promoter, a natural killer (NK) cell-specific promoter, an inducible promoter that functions within T cells or NK cells, or a constitutive promoter.

Exemplary Cells

The engineered polypeptides provided herein can be expressed in cells for which engineered polypeptide, such as CAR, expression is useful, i.e., cells are engineered to comprise an engineered polypeptide-encoding nucleic acid provided herein, such that, upon expression of the nucleic acid in the cell, the cell expresses the engineered polypeptide described herein. For example, the engineered polypeptides described herein can be expressed in T lymphocytes or natural killer cells. Cells provided herein that express the CARs described herein may be referred to as “CAR cells.”

In certain embodiments, provided herein is a cell (e.g., a T lymphocyte or a natural killer cell) that has been modified to express an engineered polypeptide comprising a degradation domain provided herein. In some embodiments, the cell has been modified to express an engineered polypeptide that is a CAR comprising (a) components of a CAR, such as an antigen-binding domain, a transmembrane domain, a cell signaling domain, and/or a co-stimulatory domain, and (b) a degradation domain. In some embodiments, the cell has been modified to express an engineered polypeptide comprising a degradation domain fused to or inserted into another protein. Contacting the modified cell with a degradation agent provided herein results in ubiquitination and degradation of the engineered polypeptide.

In some embodiments, the engineered polypeptides provided herein are expressed in T lymphocytes. The T lymphocytes can be naive T lymphocytes or MHC—restricted T lymphocytes. In certain embodiments, the T lymphocytes are tumor infiltrating lymphocytes (TILs). In certain embodiments, the T lymphocytes have been isolated from a tumor biopsy, or have been expanded from T lymphocytes isolated from a tumor biopsy. In certain other embodiments, the T lymphocytes have been isolated from, or are expanded from T lymphocytes expanded from, peripheral blood, cord blood, or lymph.

In some embodiments, the cells (e.g., T lymphocytes) engineered to comprise/express engineered polypeptide described herein are autologous to an individual to whom the cells (e.g., T lymphocytes) are to be administered as part of a method of treatment described herein. In other embodiments, the cells (e.g., T lymphocytes) engineered to comprise/express an engineered polypeptide described herein are allogeneic to an individual to whom the cells (e.g., T lymphocytes) are to be administered. Where allogeneic cells (e.g., T lymphocytes) are used to prepare the modified cells, such as CAR cells, it is preferable to select cells (e.g., T lymphocytes) that will reduce the possibility of graft-versus-host disease (GVHD) in the individual. For example, in certain embodiments, virus-specific T lymphocytes are selected for preparation of CAR T lymphocytes; such lymphocytes will be expected to have a greatly reduced native capacity to bind to, and thus become activated by, any recipient antigens. In certain embodiments, recipient-mediated rejection of allogeneic cells (e.g., T lymphocytes) can be reduced by co-administration to the host of one or more immunosuppressive agents, e.g., cyclosporine, tacrolimus, sirolimus, cyclophosphamide, or the like.

In some embodiments, T lymphocytes are obtained from an individual, optionally expanded, and then transformed with a vector encoding an engineered polypeptide provided herein, and optionally then expanded. In some embodiments, T lymphocytes are obtained from an individual, optionally expanded, and then transformed with a vector encoding an engineered polypeptide that is a CAR described herein, and optionally then expanded. Cells containing the vector can be obtained, in some embodiments, using a selectable marker. In some embodiments, T lymphocytes are obtained from an individual, optionally expanded, and then modified to insert a degradation domain into a desired endogenous protein gene such that an engineered polypeptide is expressed which comprises the degradation domain fused to or inserted within the endogenous protein. The modified T lymphocytes may be optionally further expanded.

In certain embodiments, the T lymphocytes used to express engineered polypeptides provided herein comprise native TCR proteins, e.g., TCR-α and TCR-β that are capable of forming native TCR complexes. In certain other embodiments, either or both of the native genes encoding TCR-α and TCR-β in the T lymphocytes are modified to be non-functional, e.g., a portion or all are deleted or a mutation is inserted.

In certain embodiments, the signaling domain(s) of a CAR described herein can be used to promote proliferation and expansion of cells (e.g., T lymphocytes) comprising/expressing the CAR. For example, unmodified T lymphocytes, and T lymphocytes comprising a polypeptide comprising a CD3ζ signaling domain and a CD28 co-stimulatory domain can be expanded using antibodies to CD3 and CD28, e.g., antibodies attached to beads; see, e.g., U.S. Pat. Nos. 5,948,893; 6,534,055; 6,352,694; 6,692,964; 6,887,466; and 6,905,681. Similarly, antibodies to a signaling motif can be used to stimulate proliferation of cell (e.g., T lymphocytes) comprising a CAR described herein.

In certain embodiments, an engineered polypeptide may be used as a “suicide gene” or “safety switch” that enables killing of substantially all of the cells expressing the engineered polypeptide when desired. For example, a degradation domain may be inserted into a gene that expresses an endogenous protein necessary for survival and/or for a particular activity of a cell. Contacting the cell with a degradation agent results in ubiquitination and degradation of the endogenous protein (i.e., of the engineered polypeptide comprising the degradation domain and the endogenous protein), disabling an activity of the cell or killing the cell.

### Exemplary Degradation Agents

As used herein, the term “degradation agent” refers to a molecule (e.g., a small molecule) capable of binding a degradation domain provided herein and a ubiquitin ligase, such as an E3 ligase. In some embodiments, a degradation agent binds to a degradation domain and binds to cereblon. In some embodiments, the degradation agent binds the degradation domain and the ubiquitin ligase, resulting in an association between the E3 ligase and the degradation domain. In some such embodiments, the engineered polypeptide comprising the degradation domain is ubiquitinated by the ubiquitin ligase following association mediated by the degradation agent.

In some embodiments, the degradation agent is a cereblon-binding compound. In some embodiments, the degradation agent is 3-(5-(6,7-dihydro-5H-pyrrolo[3,4-b]pyridine-6-carbonyl)-1-oxoisoindolin-2-yl)piperidine-2,6-dione (Compound B), 3-(5-((4-(2-methylpyridin-3-yl)piperazin-1-yl)methyl)-1-oxoisoindolin-2-yl)piperidine-2,6-dione (Compound C), or 3-[5-[1-(1,3-benzothiazol-6-ylmethyl)-4-piperidyl]-1-oxo-isoindolin-2-yl]piperidine-2,6-dione (Compound D).

| Compound | Structure | Name |
|---|---|---|
| B | 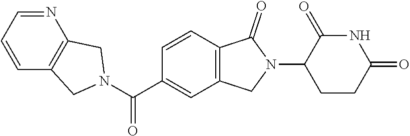 [SMILES: O=C1CCC(N2Cc3cc(C(=O)N4Cc5cccnc5C4)ccc3C2=O)C(=O)N1] | 3-(5-(6,7-dihydro-5H- pyrrolo[3,4-b]pyridine-6- carbonyl)-1-oxoisoindolin- 2-yl)piperidine-2,6-dione |
| C | 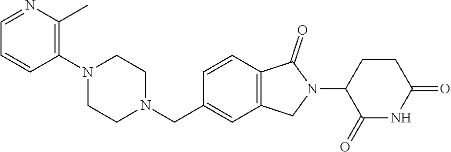 [SMILES: Cc1ncccc1N1CCN(Cc2ccc3c(c2)CN(C2CCC(=O)NC2=O)C3=O)CC1] | 3-(5-((4-(2-methylpyridin- 3-yl)piperazin-1-yl)methyl)- 1-oxoisoindolin-2- yl)piperidine-2,6-dione |
| D | 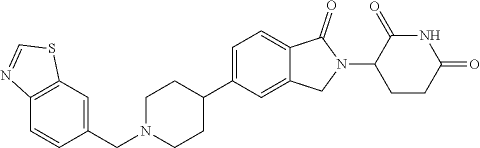 [SMILES: O=C1CCC(N2Cc3cc(C4CCN(Cc5ccc6ncsc6c5)CC4)ccc3C2=O)C(=O)N1] | 3-[5-[1-(1,3-benzothiazol-6- ylmethyl)-4-piperidyl]-1- oxo-isoindolin-2- yl]piperidine-2,6-dione |

In some embodiments, a degradation agent is a compound disclosed in WO 2019/038717 A1, which is incorporated by reference herein in its entirety.

A degradation agent used in accordance with the methods described herein, or an enantiomer or a mixture of enantiomers thereof; or a pharmaceutically acceptable salt, solvate, hydrate, co-crystal, clathrate, or polymorph thereof, can be delivered as a single dose such as, e.g., a single bolus injection, or oral tablets or pills; or over time, such as, e.g., continuous infusion over time or divided bolus doses over time.

The degradation agents used in accordance with the methods described herein can be formulated for intravenous, intraarterial, parenteral, intramuscular, subcutaneous, intrathecal, or intraocular administration, or administration within a particular organ or tissue.

### Exemplary Methods

In some embodiments, a method of reducing the level of an engineered polypeptide comprising a degradation domain is provided, comprising contacting the engineered polypeptide with a degradation agent. In some embodiments, the contacting occurs in a cell, and the degradation agent binds to the degradation domain and a ubiquitin ligase, resulting in ubiquitination and degradation of the engineered polypeptide. In some embodiments, degradation of the engineered polypeptide results in a decrease of at least one activity of the cell and/or an increase of at least one activity of the cell and/or death of the cell. Nonlimiting exemplary effects include lowing the threshold for cell (such as T cell) activation, increasing functional persistence of the cell (such as a T cell), promoting survival of the cell, and increased proliferation of the cell. In some embodiments, the degradation agent is Compound B, Compound C, or Compound D. In some embodiments, the method comprises administering the degradation agent to a subject, wherein the subject comprises cells that comprise the engineered polypeptide.

In some embodiments, the engineered polypeptide is degraded in the presence of the degradation agent. In some embodiments, the degradation agent interacts with the degradation domain and with a ubiquitin ligase, such as cereblon. In some embodiments, the degradation agent mediates a complex comprising the degradation domain, degradation agent, and the ubiquitin ligase, resulting in ubiquitination of the engineered polypeptide.

The modified cells provided herein, such as T lymphocytes (i.e., T cells) modified to comprise/express an engineered polypeptide (e.g., CAR cells), can be used to treat an individual who would benefit from the modified cells, for example, because the individual has a cancer that expresses a target of a CAR. In some embodiments, the cell is a T effector cell. In some embodiments, the cell is a CD4+ T cell or a CD8+ T cell. In some embodiments, either the T cell, T effector cell, CD4+ T cell or a CD8+ T cell comprises the engineered polypeptide.

In some embodiments, provided herein are methods for killing target cells that express an antigen bound by the antigen-binding domain of a CAR described herein, wherein said methods comprise contacting said target cells with a modified cell provided herein (e.g., a T cell or NK cell) comprising/expressing a CAR described herein. In some embodiments, said target cell is a cancer cell, e.g., a blood cancer cell or a solid tumor cell. In some embodiments, provided herein are methods of treating cancer, said methods comprising administering a population of modified cells described herein, e.g., a T cells or NK cells, that comprise/express a CAR described herein, wherein said CAR comprises an antigen-binding domain specific for a cancer antigen (e.g., TSA or TAA) to a subject.

In some embodiments, the target cell or cancer cell expresses one or more the following antigens, or a fragment thereof: 4- IBB, 5T4, 8H9, B7-H6, adenocarcinoma antigen, a-fetoprotein, B Cell Maturation Antigen (BCMA), BAFF, B-lymphoma cell, C242 antigen, CA9, carcinoembryonic antigen, CA-125, carbonic anhydrase 9 (CA-IX), CCR4, CD3, CD4, CD 19, CD20, CD22, CD23 (IgE receptor), CD28, CD30 (T FRSF8), CD33, CD38, CD40, CD44v6, CD44v7/8, CD51, CD52, CD56, CD74, CD80, CD123, CD152, CD171, CD200, CD221, CE7, CEA, C-MET, CNT0888, CTLA-4, DRS, EpCAM, ErbB2, ErbB3/4, EGFR, EGFRvIII, EphA2, EGP2, EGP40, FAP, Fetal AchR, fibronectin extra domain-B, folate receptor-a, folate receptor 1, G250/CAIX, GD2, GD3, glycoprotein 75, GP MB, HER2/neu, HGF, HLA-AI MAGE A1, HLA-A2 NY-ESO-1, HMW-MAA, human scatter factor receptor kinase, IGF-1 receptor, IGF-I, IgG1, IL-6, IL-13, IL-13 receptor a2, IL-11 receptor a, insulin-like growth factor I receptor, integrin a5I31, integrin avI33, Kappa light chain, L1-CAM, Lambda light chain, Lewis Y, mesothelin, MORAb-009, MS4A1, MUC1, MUC1 6, mucin CanAg, NCAM, N-glycolylneuraminic acid, NKG2D ligands, NPC-IC, PDGF-R a, PDL192, phosphatidylserine, prostate-specific cancer antigen (PSCA), prostatic carcinoma cells, PSMA, PSC1, RANKL, RON, ROR1, SCH 900105, SDC1, SLAMF7, sp17, TAG72, tenascin C, TGF (32, TGF-β, TL1A, TRAIL-R1, TRAIL-R2, tumor antigen CTAA16.88, VEGF-A, VEGF receptors, VEGFR-1, VEGFR2, TEM1, TEM8, and/or vimentin.

In some embodiments, following administration of modified cells provided herein, such as CAR cells, it may be desirable to reduce or eliminate expression of the CAR and thus reduce or eliminate targeted cell killing. In some such embodiments, the method may further comprise administering a degradation agent provided herein to the subject. Administration of the degradation agent results in degradation of the engineered polypeptide (e.g., the CAR), and reduces or eliminates targeting of the modified cells to cells expressing the antigen bound by the antigen-binding domain of the CAR. In this way, the activity of treatments with CAR cells may be modulated, and safety may be improved.

In some embodiments, said population of modified cells is administered first to the subject, followed by administration of the degradation agent at a specified period of time after administration of the modified cell population, e.g., 30 minutes, 1 hour, 6 hours, 12 hours, 1 day, 2 days, 3 days, 4 days, 5 days, 6 days, or 1 week after administration of the cell population.

In some embodiments, the degradation agent is Compound B, compound C, or Compound D.

A non-limiting list of cancers that can be treated in accordance with the methods of treatment described herein includes lymphoma, leukemia, lung cancer, breast cancer, prostate cancer, adrenocortical carcinoma, thyroid carcinoma, nasopharyngeal carcinoma, melanoma, skin carcinoma, colorectal carcinoma, desmoid tumor, aesmoplastic small round cell tumor, endocrine tumor, Ewing sarcoma, peripheral primitive neuroectodermal tumor, solid germ cell tumor, hepatoblastoma, neuroblastoma, non-rhabdomyosarcoma soft tissue sarcoma, osteosarcoma, retinoblastoma, rhabdomyosarcoma, Wilms tumor, glioma, glioblastoma, myxoma, fibroma, and lipoma. Exemplary lymphomas and leukemias include, without limitation, chronic lymphocytic leukemia (small lymphocytic lymphoma), B-cell prolymphocytic leukemia, lymphoplasmacytic lymphoma, Waldenstrom macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, or a non-Hodgkin lymphoma.

Efficacy of the modified cells described herein, such as CAR cells, in treatment of a disease or disorder, e.g., in treatment of an individual having cancer, can be assessed by one or more criteria specific to the particular disease or disorder, known to those of ordinary skill in the art, to be indicative of progress of the disease or disorder. Generally, administration of CAR cells (e.g., CAR T lymphocytes) to an individual having a disease/disorder (e.g., cancer) is effective when one or more of said criteria detectably, e.g., significantly, moves from a disease state value or range to, or towards, a normal value or range.

The modified cells described herein can be formulated in any pharmaceutically-acceptable solution, preferably a solution suitable for the delivery of living cells, e.g., saline solution (such as Ringer's solution), gelatins, carbohydrates (e.g., lactose, amylose, starch, or the like), fatty acid esters, hydroxymethylcellulose, polyvinyl pyrolidine, etc. Such preparations are preferably sterilized prior to addition of the CAR cells, and may be mixed with auxiliary agents such as lubricants, preservatives, stabilizers, emulsifiers, salts for influencing osmotic pressure, buffers, and coloring. Pharmaceutical carriers suitable for use in formulating CAR cells are known in the art and are described, for example, in WO 96/05309.

In certain embodiments, the modified cells (e.g., CAR cells) described herein are formulated into individual doses, wherein said individual doses comprise at least, at most, or about 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, or 1×10¹¹ cells.

In certain embodiments, the modified cells (e.g., CAR cells) described herein are formulated for intravenous, intraarterial, parenteral, intramuscular, subcutaneous, intrathecal, or intraocular administration, or administration within a particular organ or tissue.

### 7. EXAMPLES

### 7.1. Example 1: IKZF1 ZNF2 G-Motif-Tagged CARs Retain Function

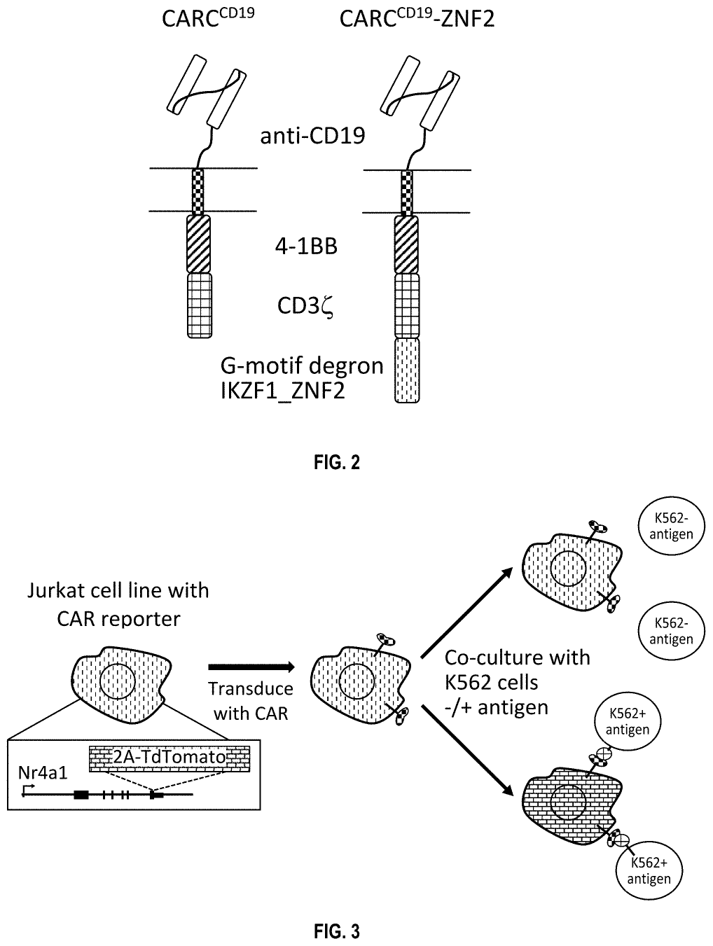

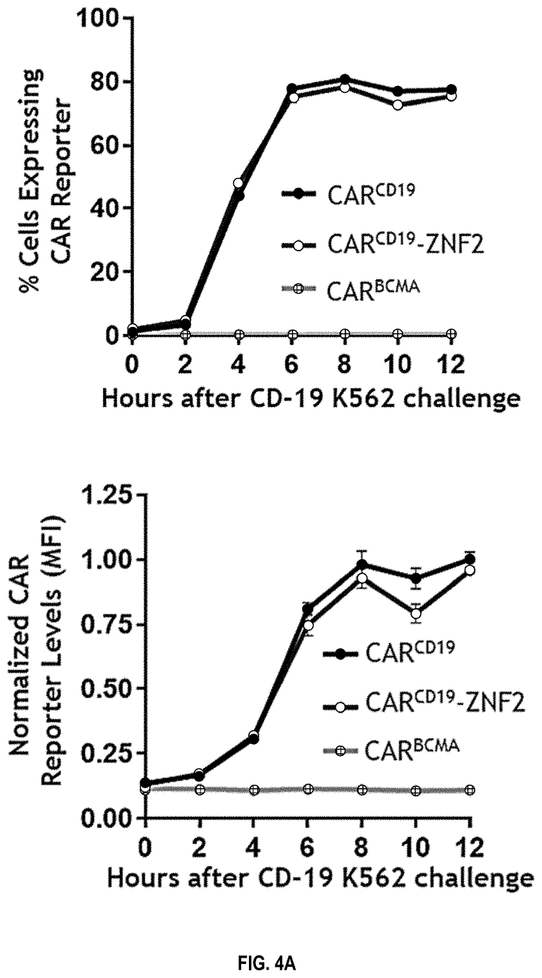

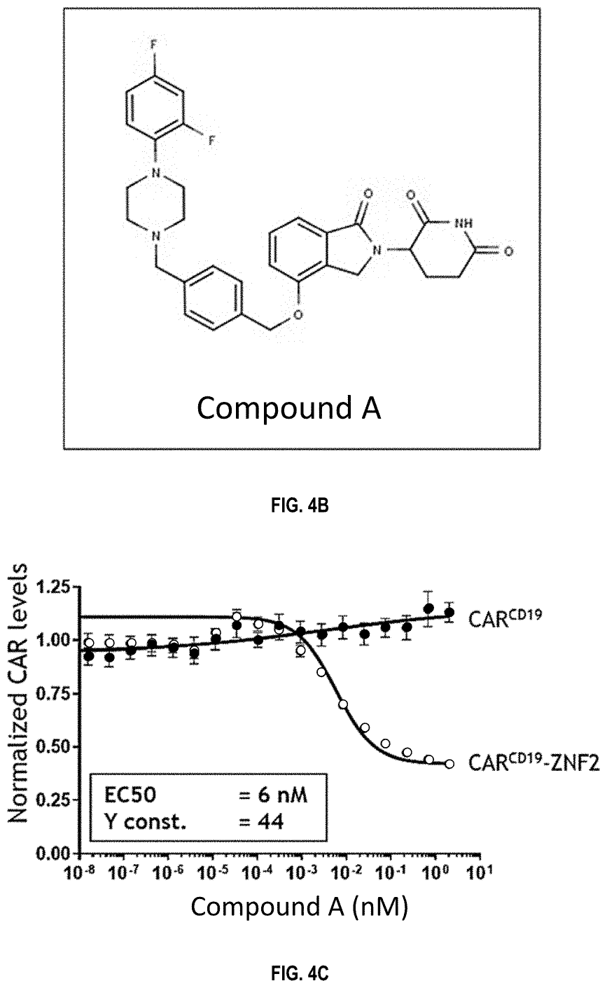

Function of IKZF1 ZNF2 tagged CD19 CAR T cells (FIG. 2, ZNF2 amino acid sequence of SEQ ID NO: 15) was evaluated in a reporter assay, in which Jurkat cells have been engineered to express tdTomato when the Nur77 gene, associated with CAR and TCR activation, is actively transcribed (FIG. 3). Lentiviral vectors encoding ZNF-tagged CD19 CAR, untagged CD19 CAR, or an untagged BCMA CAR negative control were transduced into the Jurkat reporter cell line. Transduced cells were then co-cultured with a CD19-expressing K562 target cell line at 37° C. Levels of tdTomato, both in the form of mean fluorescence intensity (MFI) and the overall percent of positive cells, were assessed by flow cytometry every 2 hours for 12 hours. The results showed that both the timing and levels of CAR activation were identical between the tagged and untagged CD19 CARs, while the BCMA CAR Ts failed to be activated in the presence of the CD19 target cell line (FIG. 4A).

To assess the level of CAR degradation achieved at different concentrations of Compound A, a drug titration of Compound A (FIG. 4B) was performed on Jurkats transduced with IKZF1 ZNF2-tagged or untagged CD19 CAR. Compound A is shown in US Publication No. 2019/0008852 A1 at page 42, Table 4 (Compound A). Cells were incubated with Compound A for 24 hours at 37° C., after which CAR levels were assessed by flow cytometry. The data show that the IKZF1 ZNF2 G-motif degron mediated ˜55% CAR degradation at high Compound A concentrations (FIG. 4C). This level of CAR degradation may not be sufficient to inhibit CAR activity when challenged with K562 expressing an antigen recognized by the CAR (data not shown). This result prompted a search to modify the degron protein sequence to enable it to respond more potently to Compound A.

### 7.2. Example 2: Evaluation of Alternate Configurations of IKZF1 Zinc Fingers

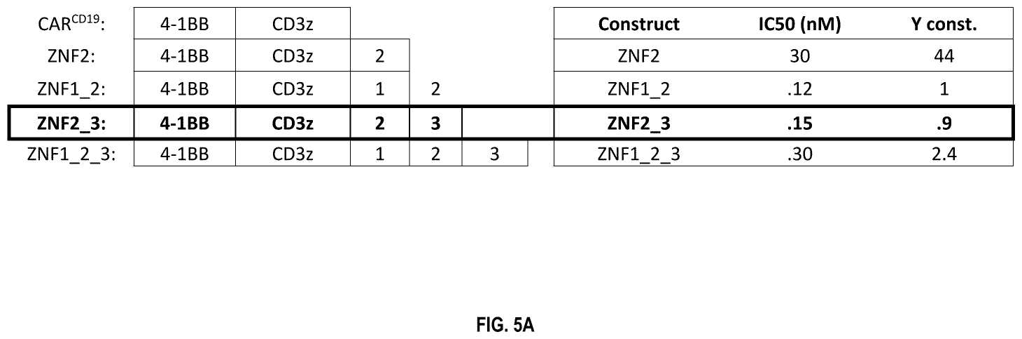

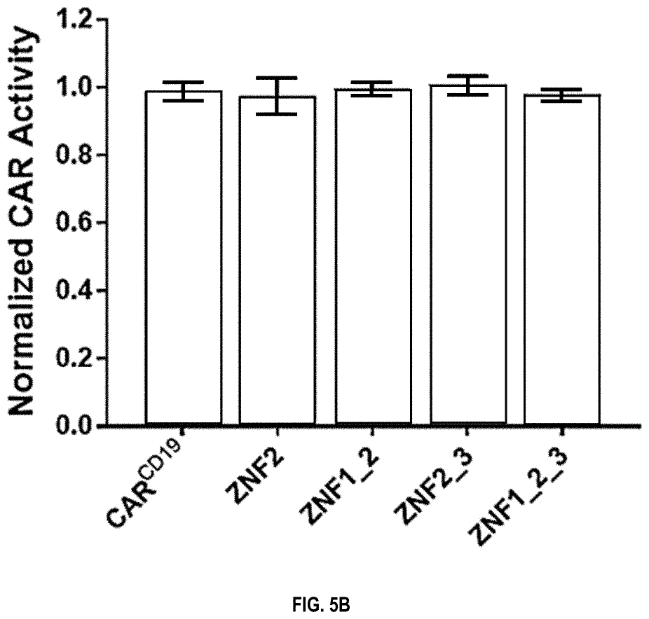

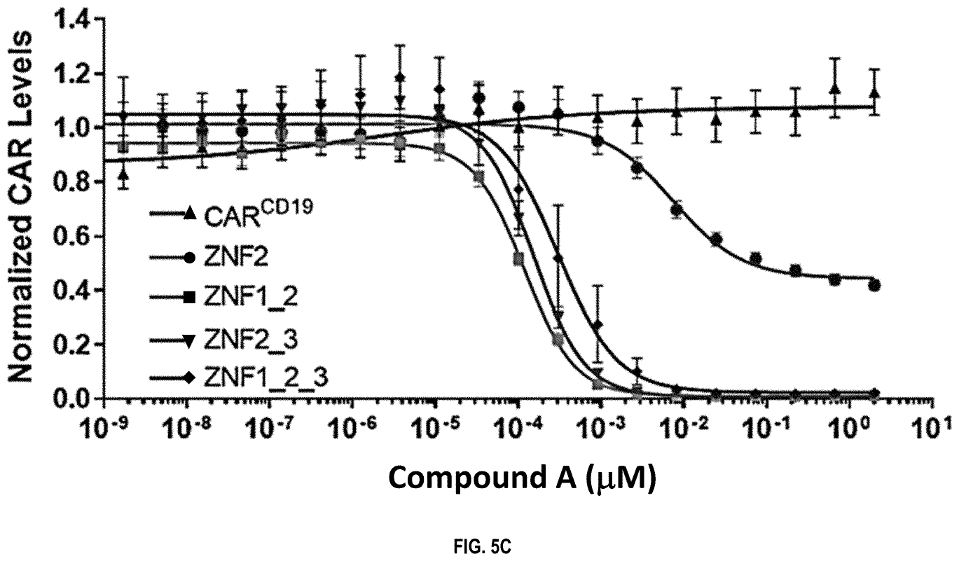

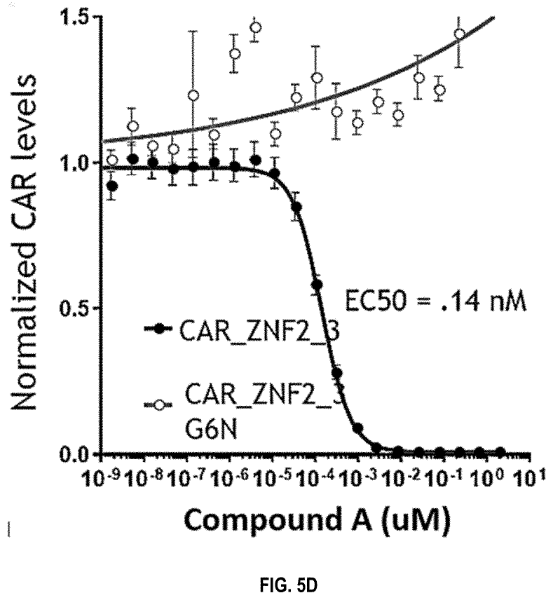

The IKZF1-ZNF2 degron was further modified to promote improved CAR degradation. Untagged CAR and CAR tagged with the original IKZF1-ZNF2 degrons were tested alongside alternate orientations and combinations of IKZF1 ZNF1, ZNF2, and ZNF3 (FIG. 5A). Each CAR included a CD28 transmembrane domain, a 4-1BB costimulatory domain, and a CD3 (CD3z) signaling domain. The IKZF1 degrons were fused to the C-terminus. See SEQ ID NOs: 53-57. A lentiviral vector encoding untagged CD19 CAR or CAR tagged with various configurations of IKZF1-ZNFs was transduced into the Jurkat cell line. Transduced cells were first co-cultured with a CD19-expressing K562 target cell line at 37° C. for 7.5 hours, and normalized levels of tdTomato reporter were assessed by flow cytometry. The results demonstrated that none of the degron tags decreased CAR activity in the presence of antigen relative to the untagged CAR (FIG. 5B). Next, CAR-transduced Jurkat cells were cultured at 37° C. for 24 hours in the presence of a titration of Compound A to determine Y-max and EC50 values for each degron. From this experiment, it was determined that ZNF2_3 tagging led to enhanced degradation at the lowest concentrations of Compound A (FIG. 5C). ZNF1 2 tagging was excluded as ZNF1 contains a G-motif and adds additional complexity to tandem degron. This configuration was therefore selected for further testing.

To demonstrate that this degradation was dependent on a functional degron, CAR tagged with WT ZNF2_3 (SEQ ID NO: 13) was tested as above with a Compound A titration alongside CAR tagged with ZNF2_3 containing a G6N mutation in the G-motif (QCNQCNASF; SEQ ID NO: 17) (FIG. 5D). While the WT degron drove deep CAR degradation at higher Compound A concentrations, the G6N mutant tagged CAR failed to respond to Compound A at any dose. These results suggest that the G6 residue of the G-motif contributes to Compound A interaction with the optimized ZNF2_3 degron.

### 7.3. Example 3: IKZF1 ZNF2_3 Tagged CAR Degradation Inhibits Activity

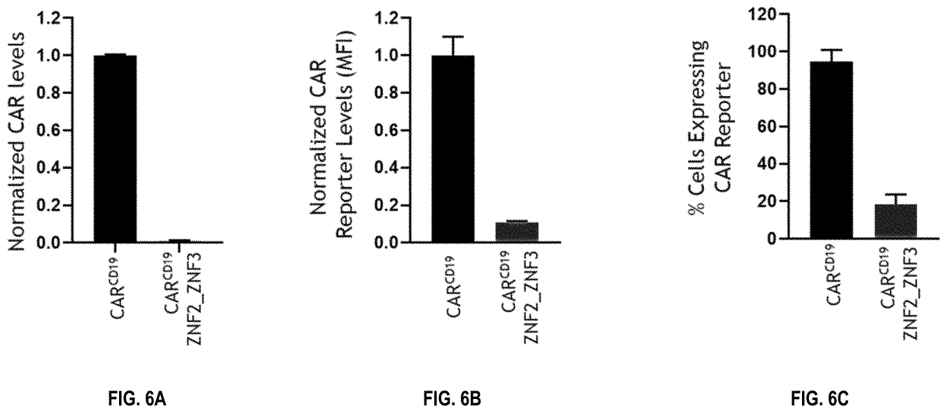

The ability to abrogate CAR T signaling by degrading IKZF1 ZNF2_3 tagged CD19 CAR T cells was evaluated in a Jurkat reporter assay. A lentiviral vector encoding ZNF2_3 tagged or untagged CD19 CAR (see Example 2) was transduced into the reporter cell line. Transduced cells were then pre-treated for 48 hours with 100 nM Compound A, then co-cultured with a CD19-expressing K562 target cell line at 37° C. for 8 hours. Levels of tdTomato in the form of mean fluorescence intensity (MFI) (FIG. 6B) and the overall percent of positive cells, as well as normalized CAR levels (FIG. 6C), were assessed by flow cytometry (FIG. 6A). At this concentration of Compound A, degradation of CARs containing the ZNF2_3 degron was sufficiently deep to strongly inhibit CAR activity when challenged with K562 cells expressing the cognate antigen.

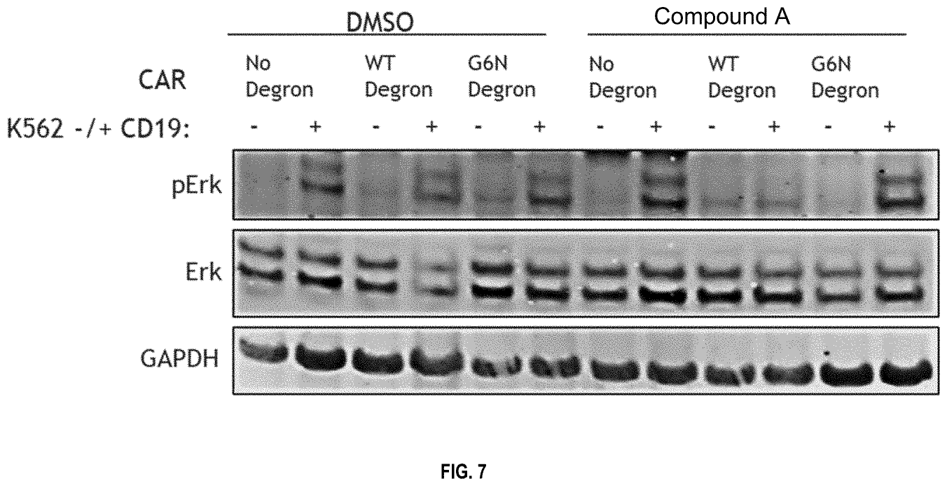

Abrogation of signaling with CAR degradation was assessed by measuring activation of the downstream MAPK signaling pathway. Jurkat cells were transduced with untagged, WT degron-tagged, or G6N degron-tagged CAR as described above. Cells were pretreated with 1 mM Compound A for 12 hours at 37° C., then co-cultured with either parental or CD19-expressing K562 cells for 30 minutes. Cells were then pelleted, lysed, run on a denaturing protein gel, transferred to a membrane, probed with antibody and visualized with film (FIG. 7). These results show that endogenous signaling pathways activated by CAR, represented here by Erk phosphorylation (pErk), are attenuated in the presence of antigen after administering Compound A to cells that contain WT IKZF1 ZNF2_3-tagged CARs.

### 7.4. Example 4: Degron Mutagenesis to Identify Novel Degron-Compound Pairs

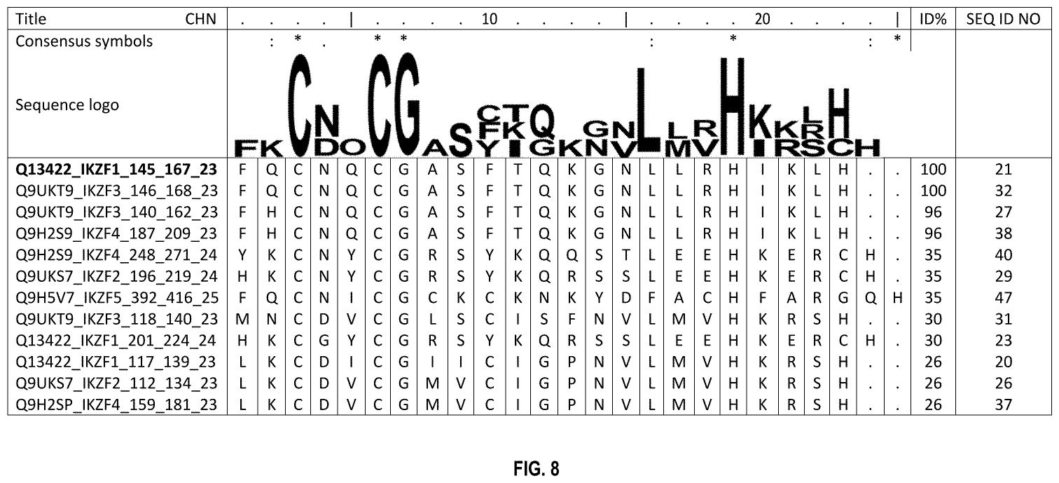

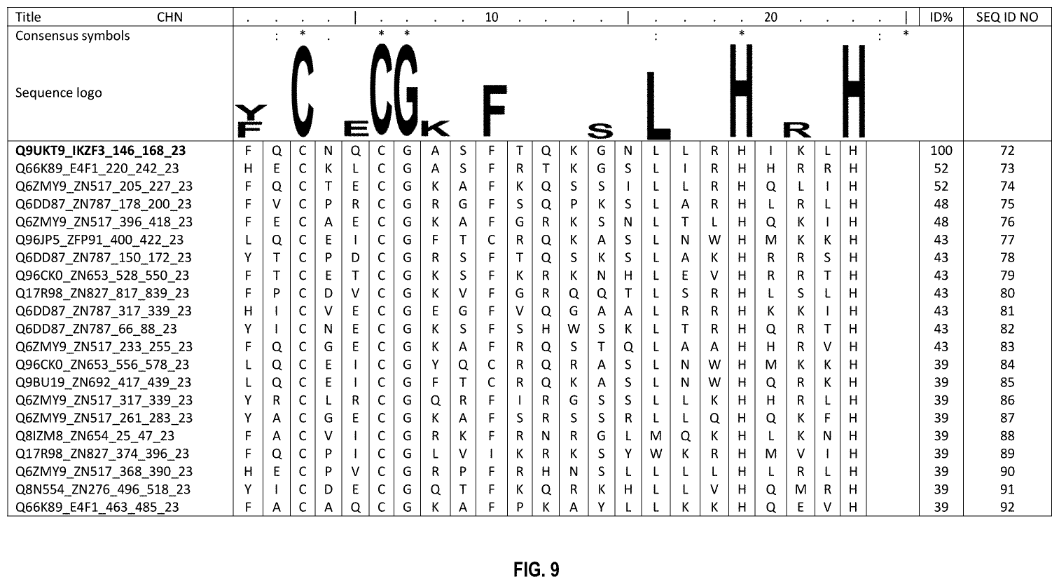

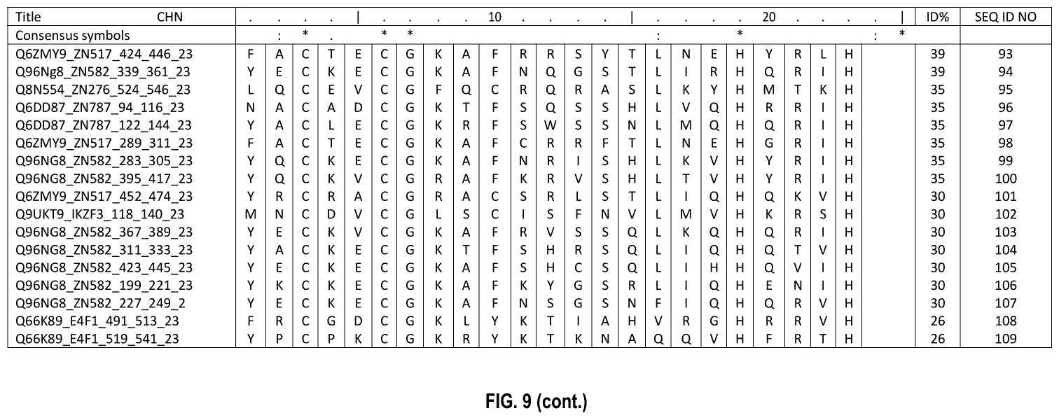

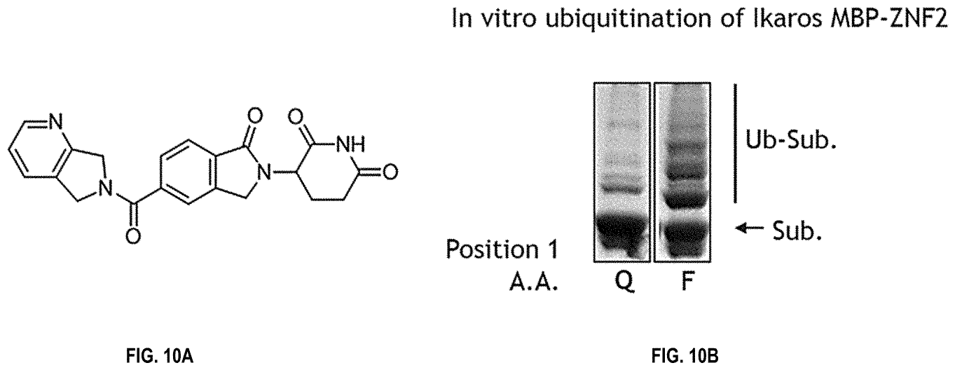

Structural studies reveal that position 1 of the G-motif is in close proximity to the compound when substrates are bound to cereblon/compound complexes (not shown). Alignment of the G-motif degron sequences of the IKZF family (FIG. 8) and predicted degrons within other C2H2 zinc fingers (FIG. 9) reveal a diverse set of amino acids at this position. Therefore, to identify additional compound:degron pairs, mutations were made in the Q1 position of IKZF1 ZNF2, and potential compounds were screened for activity against these mutants. Plasmids were built containing Ikaros MBP-ZNF2 (aa 141-196) with alternate amino acids at the Q1 position, and an in vitro ubiquitination screen was conducted using various compounds including Compound B (FIG. 10A). After treatment, cells were then pelleted, lysed, run on a denaturing protein gel, transferred to a membrane, probed with antibody and visualized with film (FIG. 10B). As shown in FIG. 10B, IKZF1 ZNF2 Q1F was heavily ubiquitinated in the presence of Compound B.

### 7.5. Example 5: Small-Molecule Screen to Identify Compounds with Potent Activity Against the Q1F Degron

A set of potential degron-targeting compounds was screened against the Q1F degron, resulting in a set of potent degron/small-molecule pairs. Lentiviral vectors containing CD19 CARs tagged with IKZF1 ZNF2_ZNF3 Q1F Nluc were transduced into Jurkat cells. The CD19 CAR was similar to the CD19 CAR in Example 2, but comprising the ZNF2_ZNF3 Q1F degron (SEQ ID NO: 19). Transduced cells were treated with a titration of each small molecule or no drug and then incubated at 37° C. for 18 hours. Cells were washed and stained with appropriate staining reagent to measure CAR levels. The cells were incubated at 4° C. in the staining reagents for 20 mins and then washed 3 times before being read on the flow cytometer. CAR levels were normalized to cells that had not been treated with drug. EC50 and Ymin values were calculated using the resulting titration curve. This identified small molecules that potently degraded the Q1F degron-tagged CARs (Table 1).

TABLE 1
Q1F-specific compounds

| Compound | Structure | EC50 (Q1F degron) | | Ymin (Q1F degron) |
|---|---|---|---|---|
| B | 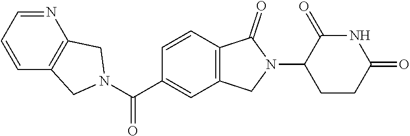 [SMILES: O=C1CCC(N2Cc3cc(C(=O)N4Cc5cccnc5C4)ccc3C2=O)C(=O)N1] | 0.0048 | uM | 1.7% |
| C | 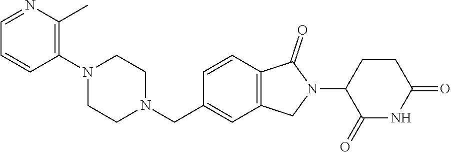 [SMILES: Cc1ncccc1N1CCN(Cc2ccc3c(c2)CN(C2CCC(=O)NC2=O)C3=O)CC1] | 0.023 | uM | 12% |
| D | 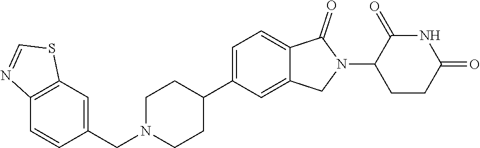 [SMILES: O=C1CCC(N2Cc3cc(C4CCN(Cc5ccc6ncsc6c5)CC4)ccc3C2=O)C(=O)N1] | 0.0007 | uM | 4.2% |

### 7.6. Example 6: IKZF1 ZNF2_3 Q1F-Tagged CARs are Degraded in a CRBN and Ubiquitin-Proteasome Pathways Dependent Manner in the Presence of Compound C

The dependence of Q1F-degron-tagged CAR degradation on CRBN and ubiquitin proteolytic pathway were assessed. CD19-targeting CAR was tagged with either IKZF1 ZNF2_3 Q1F (SEQ ID NO: 19) or Q1F/G6N (SEQ ID NO: 59). Lentiviral vectors containing these tagged CARs were then transduced into wildtype (WT) or cereblon (CRBN) knockout (KO) Jurkat cells.

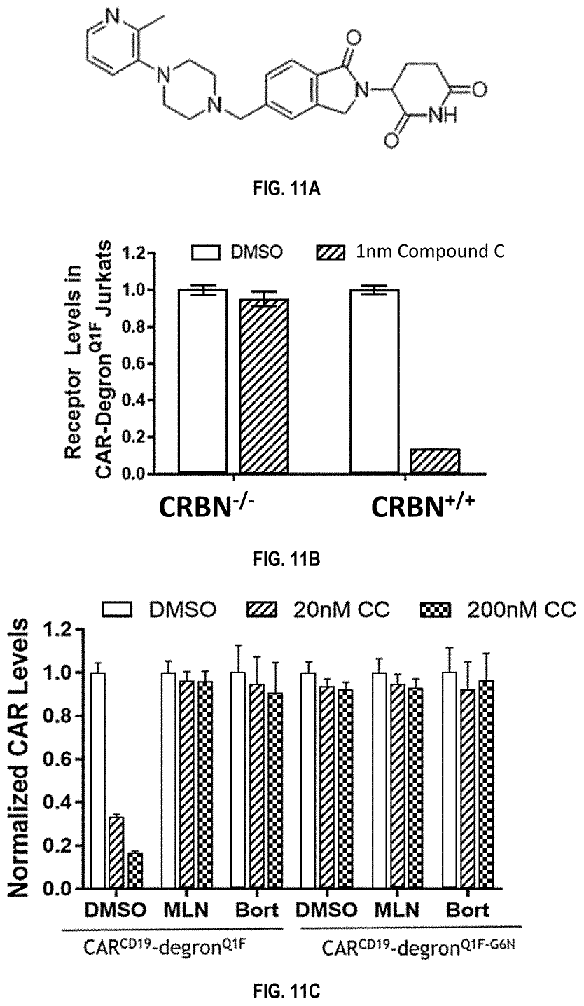

To determine whether Q1F tagged CAR can be degraded with Compound C in a CRBN-dependent manner, transduced Jurkat WT or Jurkat CRBN KO cells were treated for 1 hour with DMSO or 1 mM Compound C (FIG. 11A) at 37° C., after which CAR levels were assessed by flow cytometry. At this concentration of Compound C, tagged CAR was degraded to below 20% remaining in the WT Jurkat cells, whereas levels of tagged CAR remained identical to untagged CAR in the Jurkat CRBN KO cells (FIG. 11B). Next, dependence of CAR degradation on the ubiquitin-proteosome pathway was tested by treating WT Jurkats transduced with tagged CAR for 2 hours with DMSO, 20 nM or 200 nM Compound C alone, or alongside either 2 μM of the NEDD8 E1 enzyme inhibitor MLN4924 or 2 μM of proteasome blocker Bortezomib (FIG. 11C). The failure to degrade tagged CAR with Compound C in the presence of either inhibitor demonstrates that a functional ubiquitin proteasome pathway (UPP) is essential for Q1F-tagged CAR degradation.

### 7.7. Example 7: IKZF1 ZNF2_3 Q1F-Tagged CAR Degradation Inhibits Activity

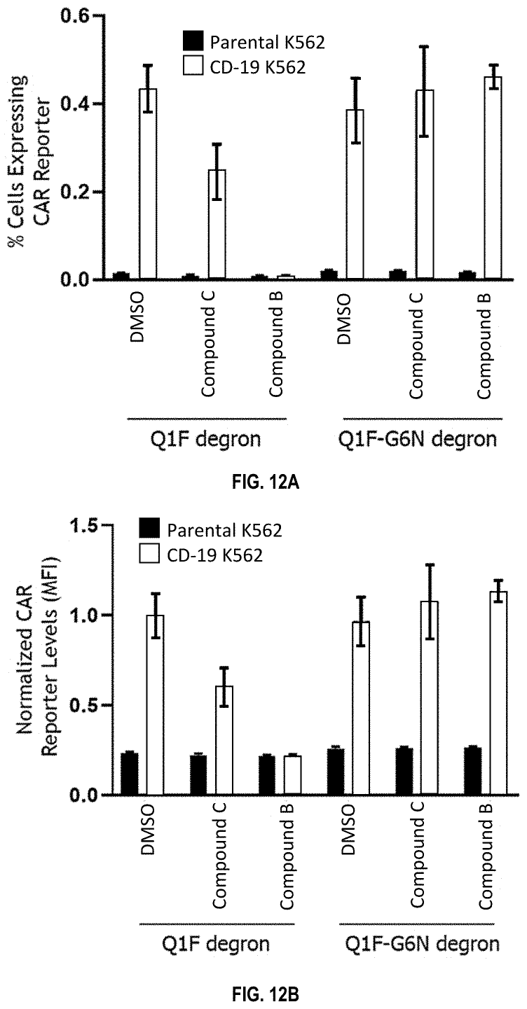

The ability to abrogate CAR T signaling by degrading IKZF1 ZNF2_3 Q1F tagged CD19 CAR T cells was evaluated in a Jurkat reporter assay (FIGS. 2 and 3). A lentiviral vector encoding ZNF2_3 Q1F or Q1F/G6N tagged CD19 CAR was transduced into the reporter cell line. Transduced cells were then pre-treated for 12 hours with 1 mM Compound C or Compound B, then co-cultured with a parental or CD19-expressing K562 target cell line at 37° C. for 8 hours. Levels of tdTomato in the form of the overall percent of positive cells (FIG. 12A), as well as mean fluorescence intensity (MFI, FIG. 12B) were assessed by flow cytometry. Degradation of CARs containing the ZNF2_3 Q1F degron decreased signaling by around 30% using 1 mM Compound C and by around 75% using Compound B; this relative difference correlates with the depth of degradation seen for each. Further, the reduction in activity depends upon having an intact G-motif, as the G6N mutation prevents inhibition.

### 7.8. Example 8: IKZF1 ZNF2_3 Q1F-Tagged CAR Degradation Inhibits Activity in Primary T Cells

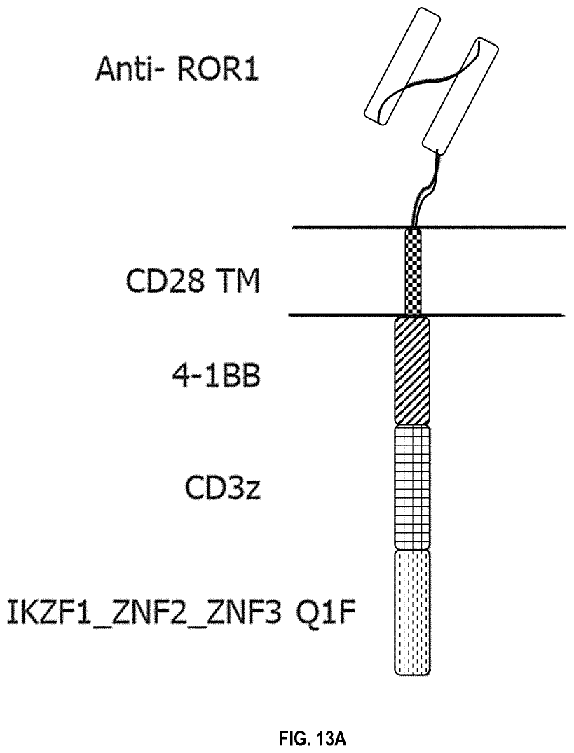

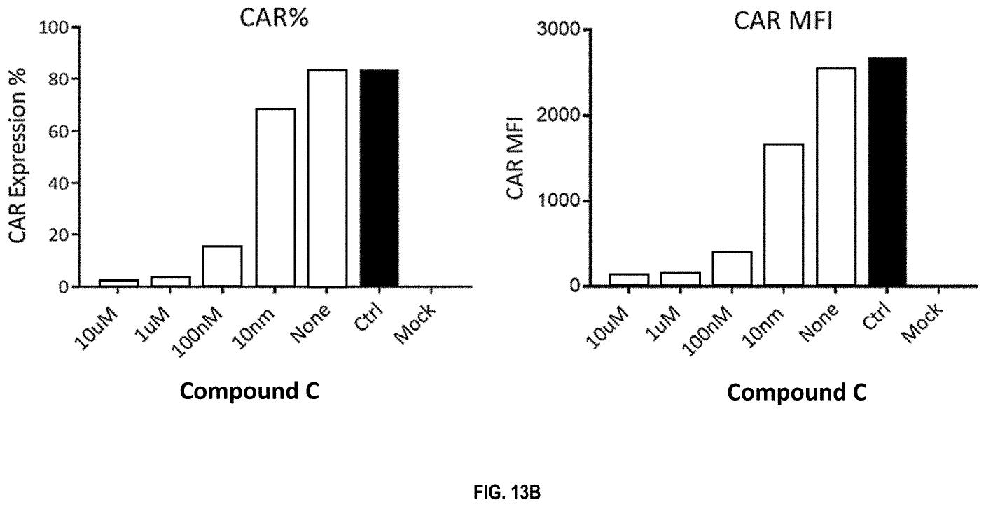

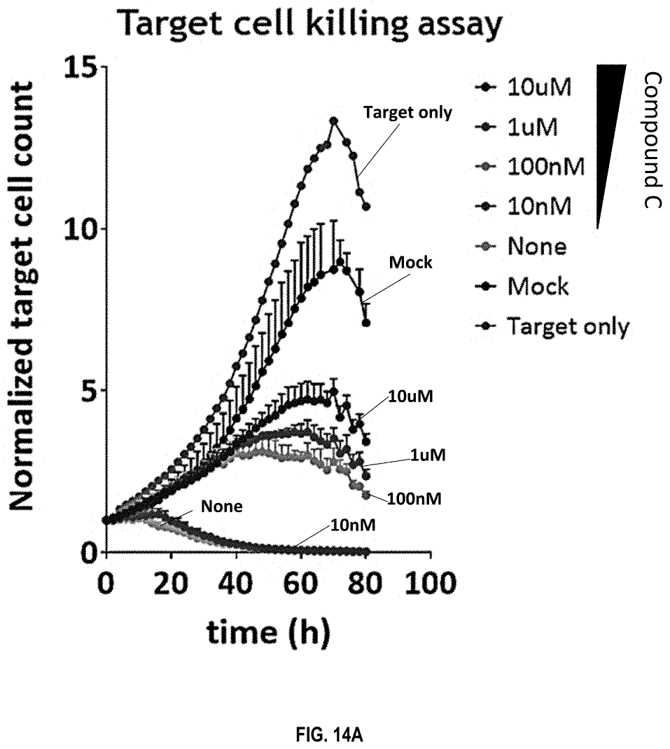

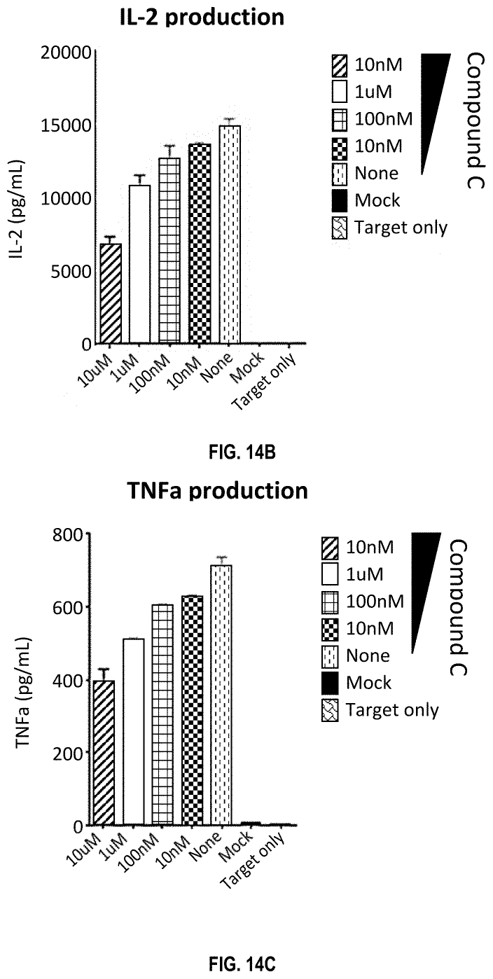

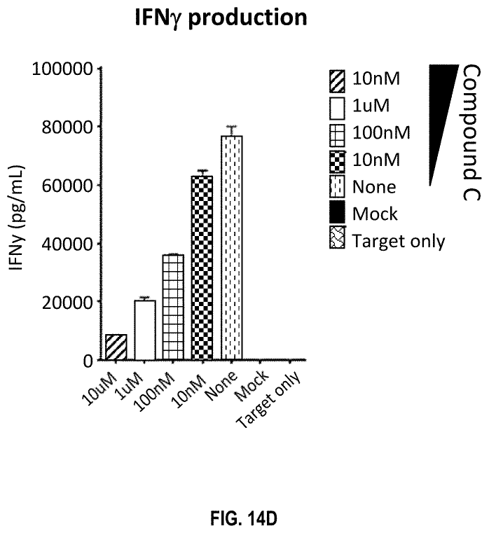

The ability to use compounds to titrate primary CAR T effector function by degrading IKZF1 ZNF2_3 Q1F tagged anti-ROR1 CAR (FIG. 13A) was evaluated. The CAR included similar transmembrane, costimulatory, and signaling domains as the CD19 CAR, but with an anti-ROR1 scFv. A lentiviral vector encoding ZNF2_3 Q1F tagged or untagged anti-ROR1 CAR was transduced into activated primary T cells. Transduced cells were expanded for 10 days in media supplemented with IL2, IL7, and IL15, then frozen. Prior to the experiment, cells were thawed and allowed to rest in media with a dose titration of Compound C or without drug. CAR levels were measured by flow cytometry (FIG. 13B), demonstrating that surface levels of ZNF2_3 Q1F-tagged CAR could be titrated with a range of Compound C concentrations. To determine the impacts of CAR degradation on CAR T activity, cells were then co-cultured with a Nuclight red labeled H-1975 ROR1 expressing target cell line at a 1:4 effector:target cell ratio. Co-cultures were placed in the Incucyte for 24 hours to monitor tumor killing via Nuclight red signal (FIG. 14A), and supernatants were collected to measure proinflammatory cytokine production by MSD (FIG. 14B-14D; IL-2, TNFα, and IFNγ). These experiments demonstrated that effector function of primary CAR T cells, as measured by both killing and cytokine production, could be titrated by targeting ZNF2_3 Q1F-tagged CAR for degradation with different concentrations of Compound C.

### 7.9. Example 9: CAR Degradation via Q1F Degron Preserves CAR Function During In Vitro Chronic Antigen Exposure

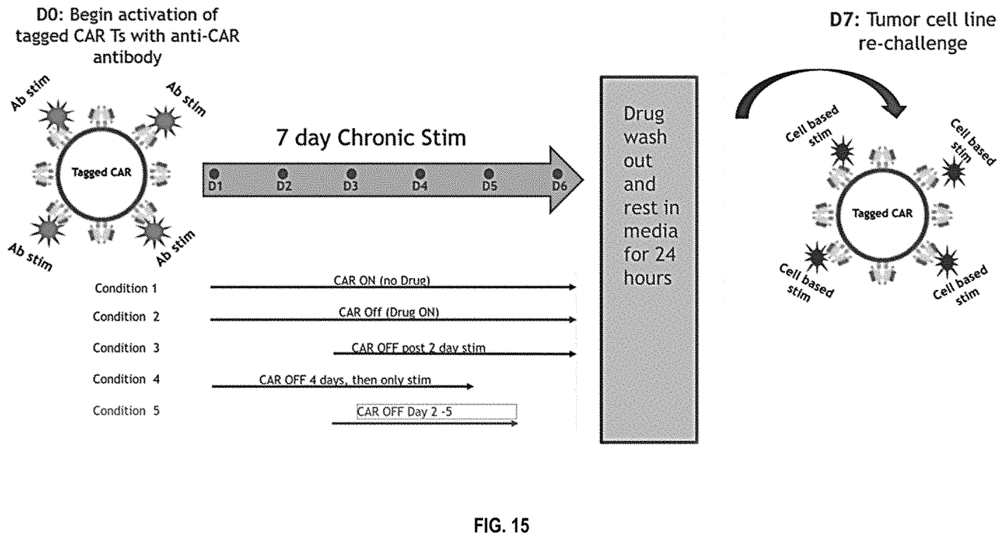

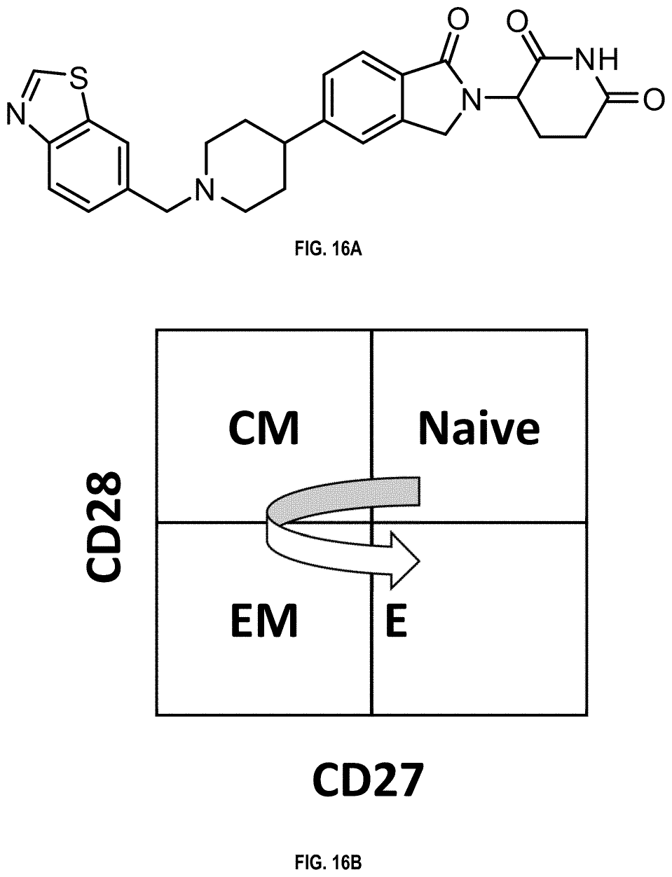

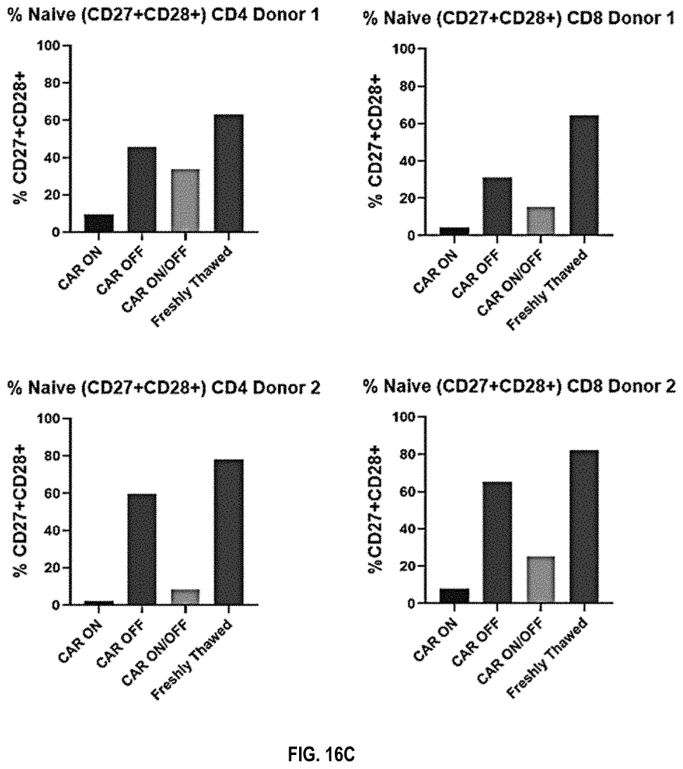

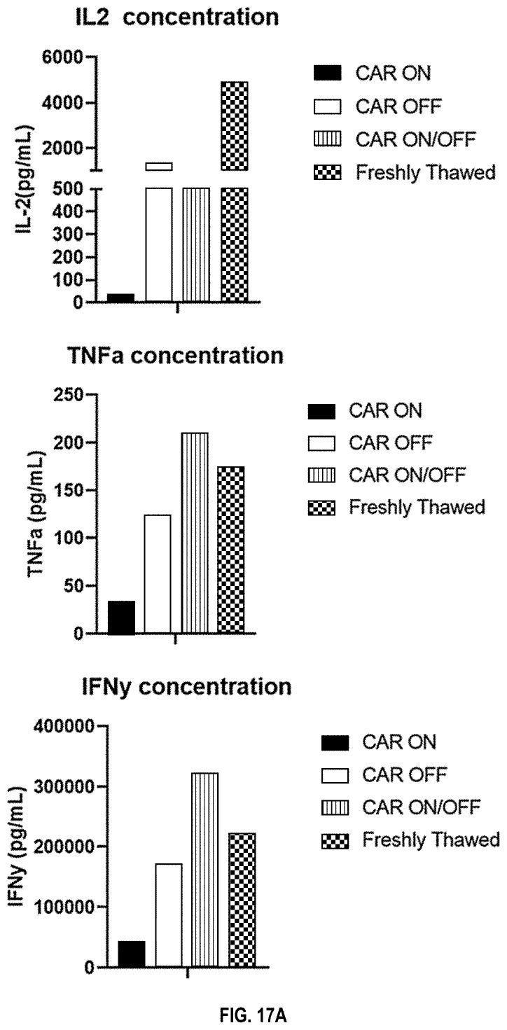

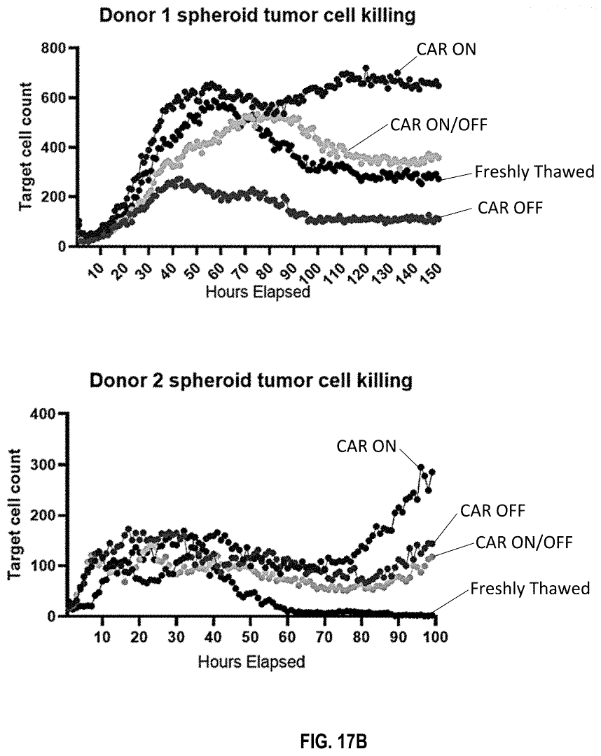

The functional impact of providing a transient rest to primary T cells during chronic antigen exposure through degradation ZNF2_3 Q1F tagged CD19 CAR was tested in vitro. Stimulation was provided by plates coated with an antibody directed against the CAR scFv, and the various durations of rest were evaluated (FIG. 15). Cells were treated with 3.9 nM of Compound D (FIG. 16A), then washed and rested in media containing to allow CAR to rebound full prior to rechallenge. Rested cells were analyzed by Flow Cytometry to assess CD27 and CD28 levels as a proxy for the naïve-like population (FIGS. 16B and 16C). The CAR T cells were then challenged with a Nuclight red labeled tumor cell line in a spheroid (3D) format, and supernatants were collected to measure proinflammatory cytokine production using a Meso Scale Discovery ELISA assay. These experiments show that the providing CAR T cells a period of transient rest leads to less activation and maintains a more naïve like population as compared to CAR T cells which undergo continuous antigen exposure. This period of transient rest also provides a functional benefit in terms of the production of pro inflammatory cytokines like IL-2, TNFα and IFNγ (FIG. 17A) and anti-tumor function (FIG. 17B); progressive loss of these cytokines and of cytotoxicity is a hallmark of T cell exhaustion.

### 7.10. Example 10: CAR Degradation via Q1F Degron Preserves CAR Function During in Vitro Chronic Antigen Exposure

The ability to degrade ZNF2_3 Q1F tagged CAR in vivo was evaluated. A lentiviral vector encoding ZNF2_3 Q1F tagged CD19 CAR was transduced into activated primary T cells. Transduced cells were expanded for 10 days in media supplemented with IL2, IL7 and IL15, then frozen.

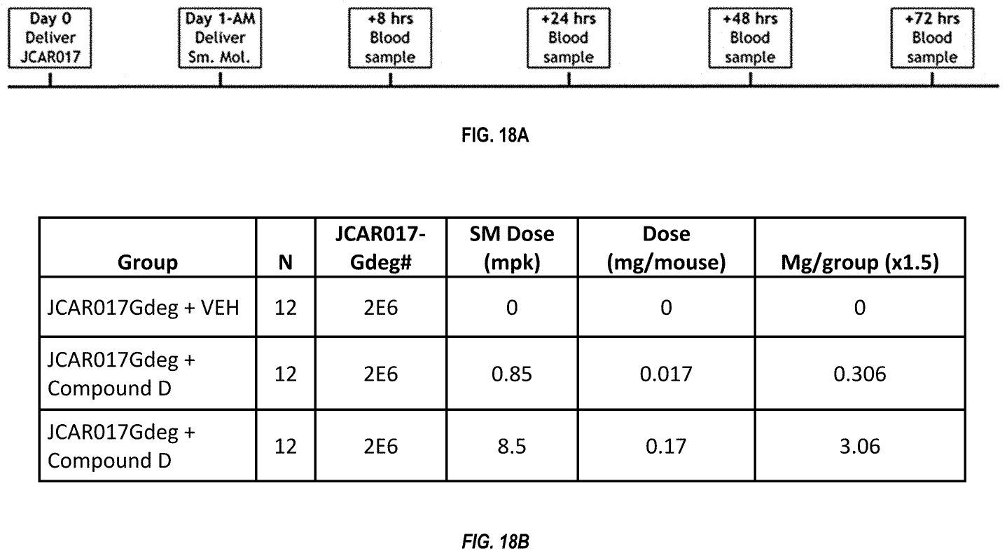

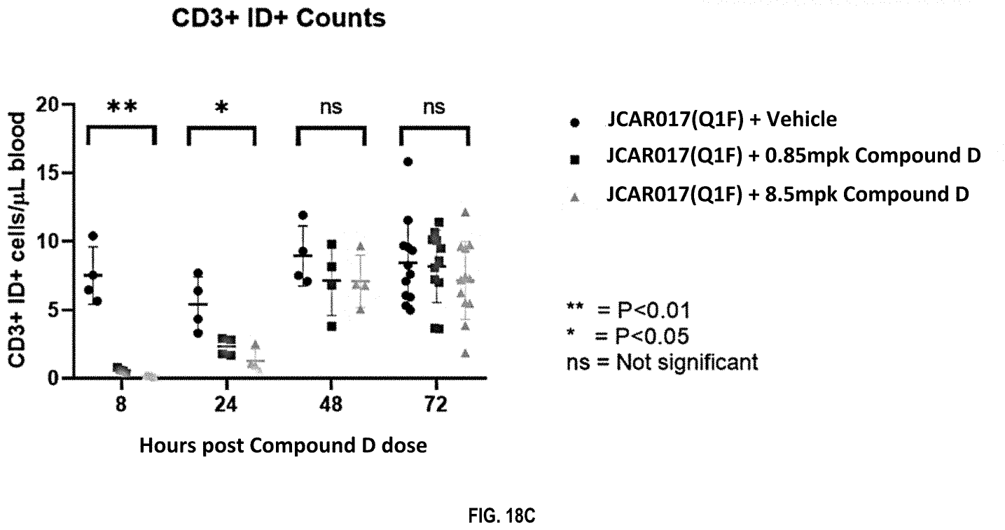

To determine the ability to degrade degron-tagged CAR in vivo in the absence of tumor, CAR T cells were thawed, rested for 24 hours and adoptively transferred into non-tumor bearing female NSG™ immunodeficient mice at a dose of 2×10⁶ cells per animal. After 24 hours, mice were dosed orally with vehicle, 0.85 or 8.5 mg/kg Compound D (FIG. 18B). Blood was drawn 8, 24, 48 and 72 hours later (FIG. 18A), and the proportion of CAR+ T cells, as determined by staining with anti-scFv and anti-CD3 antibodies, was evaluated by flow. At both doses, fewer than 5% of the CD3+ CAR+ cells remained at 8 hours, and even at 24 hours CAR remained significantly degraded (FIG. 18C), demonstrating that Q1F degron-tagged CAR can be efficiently degraded in vivo. By 48 hours after dosing, CAR expression was substantially restored (FIG. 18C).

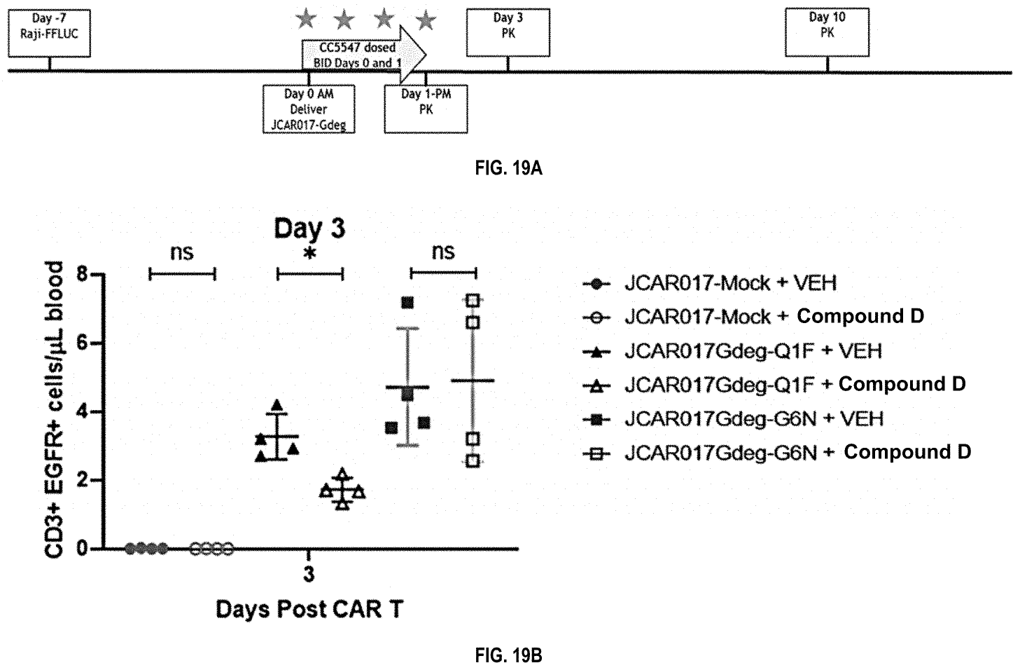

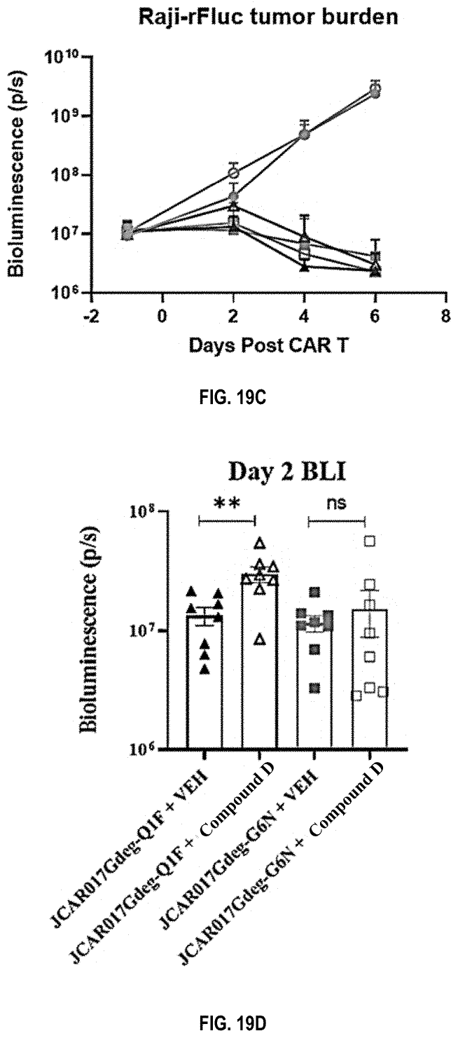

To examine the down-regulation of degron-tagged CAR in vivo in a xenograft cancer model and the impact on CAR T function, female NSG™ mice were injected with 5×10⁵ of Raji tumor cells stably expressing renilla luciferase. Six days later CAR T cells were thawed, rested for 24 hours and adoptively transferred into the mice at a dose of 2×10⁶ cells per animal. Mice were dosed orally with vehicle or 6.85 mg/kg Compound D BID on days 0 and 1 (FIG. 19A). Blood was drawn and tumor fluorescence measured at D1, D3, and D10 (FIG. 19A), and the proportion of CAR+T cells, as determined by staining with anti-scFv and anti-CD3 antibodies, was evaluated by flow. CAR degradation decreased expansion and cytolytic function of degron-tagged CAR T cells (FIG. 19B-D), showing the ability of CAR cycling to functionally rest CAR T cells.

### 7.11. Example 11: in-Frame Degron Tag Knock-In Allows Compound-Mediated Control of Endogenous Protein Levels

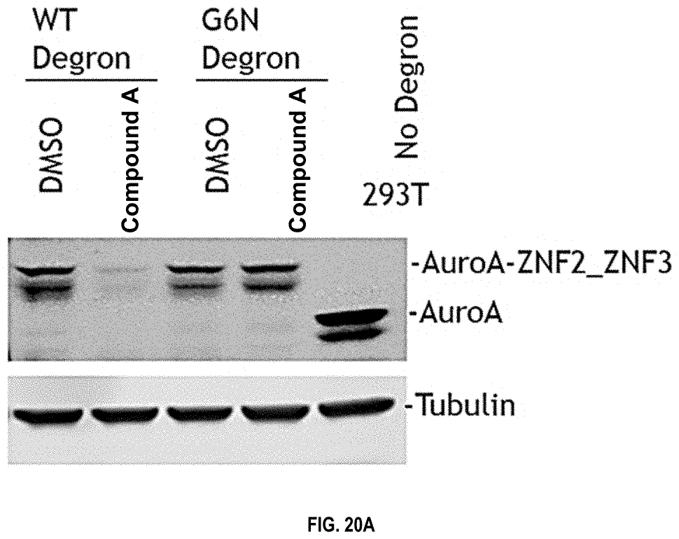

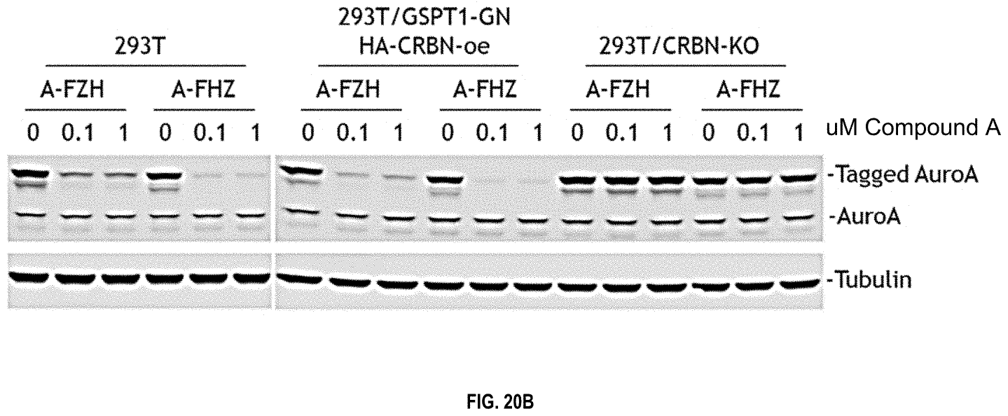

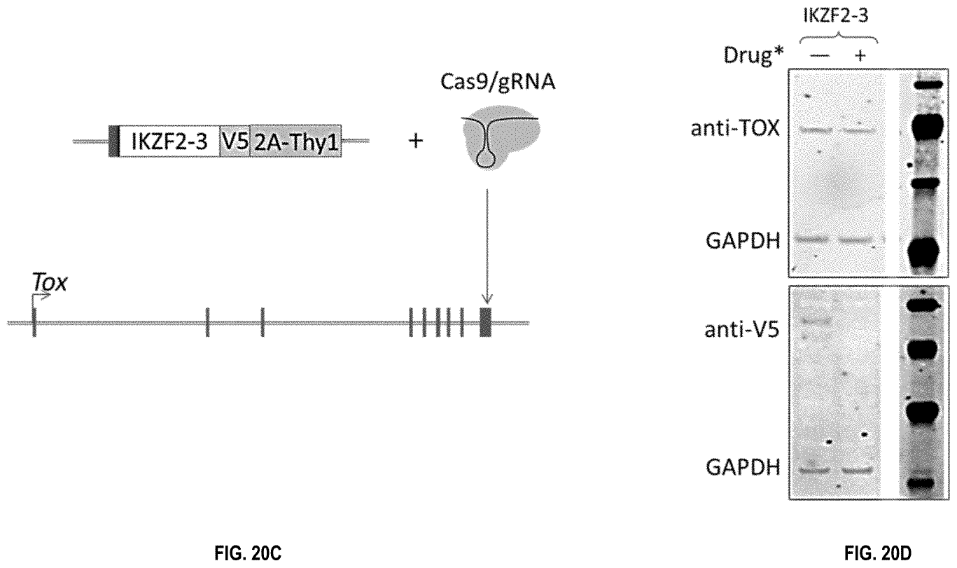

The ability to knock degron tags in-frame into genomic loci to allow for compound-mediated modulation of endogenous protein levels was explored. Adeno-associated viral vectors were designed to deliver IKZF1 ZNF2_3-V5 tag-T2A-muThy1.1 tags to be knocked in-frame into the AURA (FIG. 20A-10B) or TOX locus (FIG. 20C-20D) in Jurkat cells in both the N- and C-terminal orientations. Jurkats were then electroporated with Cas9/guide RNA ribonucleoproteins. After 5 days, knock-in cells were incubated with 1 μM Compound A or DMSO for 16 hours. Cells were then pelleted, lysed, run on a denaturing protein gel, transferred to a membrane, probed with antibody and visualized with film. Western blotting showed both Aurora A (FIG. 20A-20B) and tagged TOX (FIG. 20D) levels to be decreased in the presence of Compound A, indicating that tagging endogenous proteins with degrons allows for small-molecule control of protein levels.

### 7.12. Example 12: Synthesis of 3-(5-(6,7-dihydro-5H-pyrrolo[3,4-b]pyridine-6-carbonyl)-1-oxoisoindolin-2-yl)piperidine-2,6-dione (Compound B)

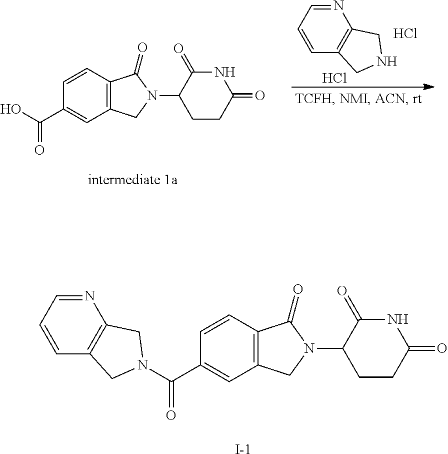 [SMILES: O=C1CCC(N2Cc3cc(C(=O)O)ccc3C2=O)C(=O)N1 ; c1cnc2c(c1)CNC2 ; Cl ; Cl ; O=C1CCC(N2Cc3cc(C(=O)N4Cc5cccnc5C4)ccc3C2=O)C(=O)N1 ; [I-]]

To a solution of 2-(2,6-dioxo-3-piperidyl)-1-oxo-isoindoline-5-carboxylic acid (400 mg, 1.4 mmol) in CH3CN (ACN) (5 mL) was added N,N,N′,N′-tetramethylchloroformamidinium hexafluorophosphate (TCFH) (779 mg, 2.8 mmol), N-methylimidazole (NMI) (1.2 g, 14 mmol) and 6,7-dihydro-5H-pyrrolo[3,4-b]pyridine; dihydrochloride (349 mg, 1.8 mmol). Then the mixture was stirred at room temperature for 1 hour. The reaction was monitored by LCMS. The precipitated solids were collected by filtration. The crude product was purified by Prep-HPLC to afford 3-[5-(5,7-dihydropyrrolo[3,4-b]pyridine-6-carbonyl)-1-oxo-isoindolin-2-yl]piperidine-2,6-dione (438.7 mg, 79% yield) as a grey solid. Analytical LC/MS (Method 5): MS[M+H⁺] 391.1. ¹H NMR (300 MHz, DMSO-d₆): δ 11.03 (s, 1H), 8.54-8.43 (m, 1H), 7.94-7.65 (m, 4H), 7.41-7.23 (m, 1H), 5.23-5.11 (m, 1H), 5.00-4.75 (m, 4H), 4.62-4.36 (m, 2H), 3.04-2.84 (m, 1H), 2.69-2.56 (m, 1H), 2.50-2.36 (m, 1H), 2.12-2.00 (m, 1H).

### 7.13. Example 13: Synthesis of 3-[5-(bromomethyl)-1-oxo-isoindolin-2-yl]piperidine-2,6-dione (Intermediate 1c)

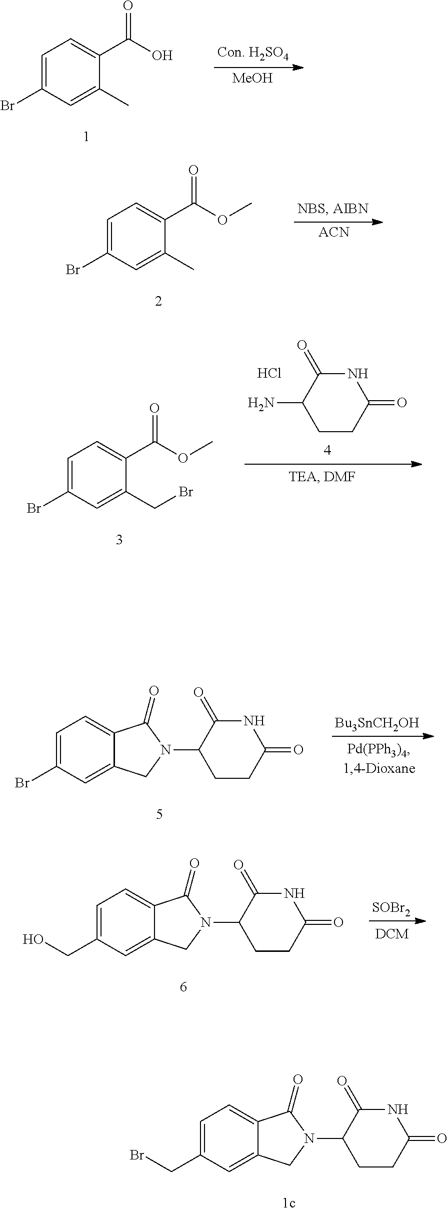

Synthesis of Compound 2

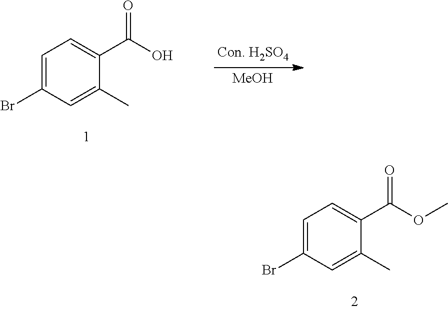

To a solution of 4-bromo-2-methyl-benzoic acid (120 g, 558 mmol) in methanol (1.0 L) was added concentrated sulfuric acid (109 g, 1.12 mol, 60 mL) between 20-40° C., then the mixture was heated to 65° C. for 18 h. TLC (petroleum ether/ethyl acetate=3:1, R_{f} (reactant)=0.1, R_{f} (product)=0.4) indicated that the reaction was finished. The reaction mixture was concentrated; the residue was separated between aqueous phase and organic layer. The aqueous phase was extracted with ethyl acetate (1 L×3). The combined organic layers were washed by saturated aqueous sodium bicarbonate (500 mL), brine (500 mL) and dried over anhydrous sodium sulfate. The mixture was filtered, and filtrate was concentrated to give methyl 4-bromo-2-methyl-benzoate (122 g, 95% yield) as yellow oil. ¹H NMR (400 MHz, CDCl₃): δ 7.76 (d, J=8.4 Hz, 1H), 7.40 (s, 1H), 7.35 (dd, J=8.4 Hz, 1.6 Hz, 1H), 3.87 (s, 3H), 2.56 (s, 3H)

Synthesis Compound 3

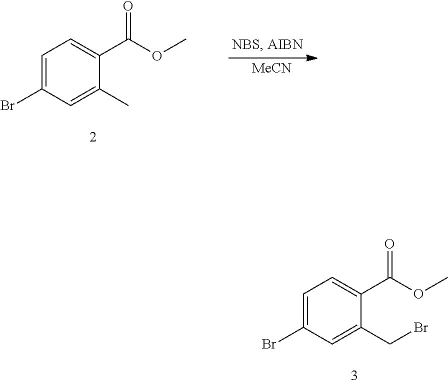

To a solution of methyl 4-bromo-2-methyl-benzoate (122 g, 533 mmol) in acetonitrile (1.20 L) was added 2, 2′-azobis(2-methylpropionitrile) (6.12 g, 37.3 mmol), the reaction mixture was heated to 82° C., N-bromosuccinimide (142 g, 799 mmol) was added in portions and stirred for 3 h. LCMS indicated that the reaction was finished. The reaction was concentrated under vacuum (50° C.). The residue was suspended in petroleum ether/dichloromethane (20:1, 100 mL), filtered and filtrate was concentrated to give crude product 150 g, which was used directly in the next step without further purification.

Synthesis of Compound 5

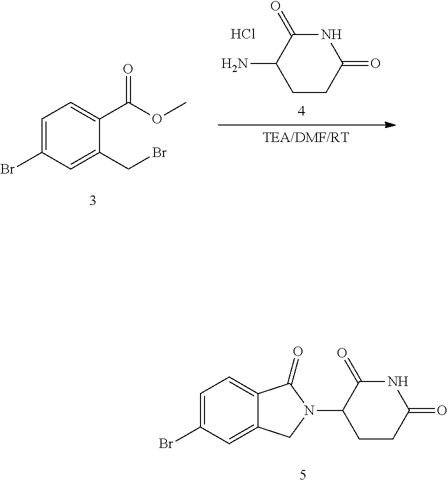 [SMILES: COC(=O)c1ccc(Br)cc1CBr ; Cl ; NC1CCC(=O)NC1=O ; O=C1CCC(N2Cc3cc(Br)ccc3C2=O)C(=O)N1]

To a solution of methyl methyl 4-bromo-2-(bromomethyl)benzoate (115 g, 375 mmol) and 3-aminopiperidine-2,6-dione (61.7 g, 375 mmol, HCl salt) in N,N-dimethylformamide (80 mL) was added N,N-diisopropylethylamine (145 g, 1.13 mol). The mixture was stirred at 50° C. for 16 h. HPLC showed the majority starting material was consumed. To the reaction mixture was added acetic acid (150 mL) and stirred for 1 h at 50° C. HPLC showed the intermediate was consumed completely. The reaction was cooled to 20° C. and filtered, filter cake was washed with water (200 mL) and ether acetate (200 mL) to give 3-(5-bromo-1-oxo-isoindolin-2-yl)piperidine-2,6-dione (91 g, 75.1% yield) as an off-blue solid. ¹H NMR (400 MHz, DMSO-d₆). δ 11.01 (s, 1H), 7.89 (s, 1H), 7.73-7.66 (m, 2H), 5.14-5.09 (m, 1H), 4.47 (d, J=16 Hz, 1H), 4.34 (d, J=16 Hz, 1H), 2.92-2.89 (m, 1H), 2.73-2.58 (m, 1H), 2.41-2.37 (m, 1H), 2.03-1.99 (m, 1H).

Synthesis of Compound 6

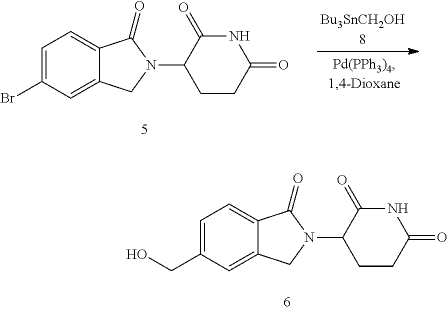 [SMILES: O=C1CCC(N2Cc3cc(Br)ccc3C2=O)C(=O)N1 ; "Bu3SnCH2OH8" ; O=C1CCC(N2Cc3cc(CO)ccc3C2=O)C(=O)N1]

To a solution of 3-(5-bromo-1-oxo-isoindolin-2-yl)piperidine-2,6-dione (80 g, 248 mmol) in dry dioxane (20 mL) was added tributylstannylmethanol (87 g, 272 mmol), the mixture was stirred at 25° C., added tetrakis(triphenylphosphine)palladium(0) (28.6 g, 24.8 mmol) to the reaction under nitrogen. The reaction mixture was stirred at 100° C. for 16 h. LCMS indicated that the reaction was finished. The reaction was concentrated and washed by dichloromethane/methanol (10:1, 250 mL×2) to give 3-[5-(hydroxymethyl)-1- oxo-isoindolin-2-yl]piperidine-2,6-dione(62 g, 91.3% yield) as a brown solid. ¹H NMR (400 MHz, DMSO-d₆): δ 10.96 (s, 1H), 7.64 (d, J=8.0 Hz, 1H), 7.52 (s, 1H), 7.42 (d, J=7.6 Hz, 1H), 5.73 (s, 1H), 5.10-5.06 (m, 1H), 4.59 (d, J=6.0 Hz, 2H), 4.35 (dd, J=17.2 Hz, 54 Hz, 2H), 2.91-2.85 (m, 1H), 2.59-2.55 (m, 1H), 2.38-2.35 (m, 1H), 2.02-1.99 (m, 1H)

Synthesis of Compound 1c

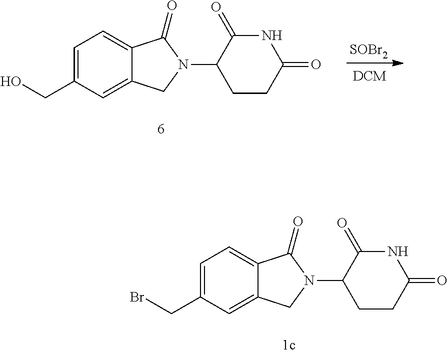

To a suspension of 3-[5-(hydroxymethyl)-1-oxo-isoindolin-2-yl]piperidine-2,6-dione (62 g, 226 mmol) in dichloromethane (1.5 L) was added sulfinyl bromide (70.5 g, 339 mmol). The reaction mixture was stirred at 18° C. for 16 h. HPLC indicated that the reaction was finished. The reaction mixture was filtered, the solid was washed with methanol (150 mL×2) to give 3-[5-(bromomethyl)-1-oxo-isoindolin-2-yl]piperidine-2,6-dione (45.0 g, 59.0% yield) as off-brown solid. ¹H NMR (400 MHz, DMSO-d₆): δ 10.97 (s, 1H), 7.70-7.66 (m, 2H), 7.56 (d, J=8.0 Hz, 1H), 5.10-5.06 (m, 1H), 4.80 (s, 2H), 4.36 (dd, J=54 Hz, 17.2 Hz, 2H), 2.91-2.85 (m, 1H), 2.59-2.49 (m, 1H), 2.47-2.35 (m, 1H), 2.00-1.98 (m, 1H)

### 7.14. Example 14: Synthesis of 3-(5-((4-(2-methylpyridin-3-yl)piperazin-1-yl)methyl)-1-oxoisoindolin-2-yl)piperidine-2,6-dione (Compound C)

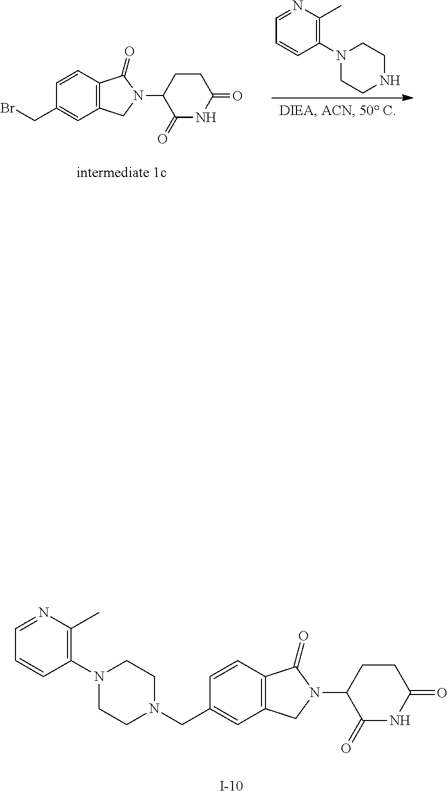

To a stirred solution of 3-[5-(bromomethyl)-1-oxo-isoindolin-2-yl]piperidine-2,6-dione (400 mg, 1.2 mmol) in MeCN (ACN) (5 mL) was added 1-(2-methyl-3-pyridyl)piperazine (315 mg, 1.8 mmol) and DIEA (460 mg, 3.6 mmol). The mixture was stirred at 50° C. for 3 h. The reaction was monitored by LCMS. The mixture was concentrated and the residue was purified by Prep-HPLC to give 3-[5-[[4-(2-methyl-3-pyridyl)piperazin-1-yl]methyl]-1-oxo-isoindolin-2-yl]piperidine-2,6-dione (187.9 mg, 34% yield) as a grey solid. Analytical LCMS (Method 1): MS[M+H⁺] 434.2. ¹H NMR (400 MHz, Methanol-d₄): δ 8.40 (d, J=5.5 Hz, 1H), 8.18 (d, J=8.2 Hz, 1H), 7.93 (d, J=7.8 Hz, 1H), 7.83-7.76 (m, 2H), 7.72 (d, J=7.6 Hz, 1H), 5.23-5.12 (m, 1H), 4.90-4.80 (m, 4H), 4.65-4.50 (m, 4H), 3.54 (s, 4H), 2.98-2.85 (m, 1H), 2.84-2.75 (m, 1H), 2.74 (s, 3H), 2.60-2.44 (m, 1H), 2.24-2.14 (m, 1H)

### 7.15. Example 15: Synthesis of 3-[5-[1-(1,3-benzothiazol-6-ylmethyl)-4-piperidyl]oxo-isoindolin-2-yl]piperidine-2,6-dione (Compound D)

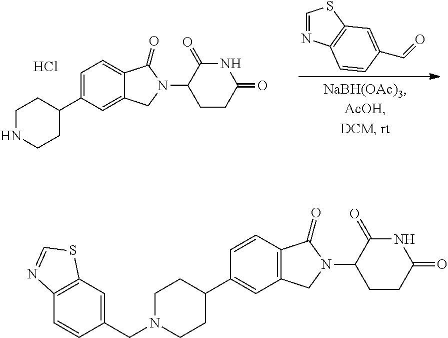 [SMILES: O=C1CCC(N2Cc3cc(C4CCNCC4)ccc3C2=O)C(=O)N1 ; Cl ; O=Cc1ccc2ncsc2c1 ; O=C1CCC(N2Cc3cc(C4CCN(Cc5ccc6ncsc6c5)CC4)ccc3C2=O)C(=O)N1]

To a stirred solution of 3-[1-oxo-5-(4-piperidyl)isoindolin-2-yl]piperidine-2,6-dione; hydrochloride (1.5 g, 4.12 mmol) in DCM (20 mL) was added 1,3-benzothiazole-6-carbaldehyde (1.35 g, 8.25 mmol) and NaBH(OAc)₃ (2.62 g, 12.37 mmol). The mixture was stirred at room temperature for overnight. The mixture was concentrated and purified by prep-HPLC to give 3-[5-[1-(1,3-benzothiazol-6-ylmethyl)-4-piperidyl]-1-oxo-isoindolin-2-yl]piperidine-2,6-dione (968.9 mg, 2.0233 mmol, 49.076% yield) as a white solid. LCMS: 475.1 [M+1]⁺. ¹H NMR (300 MHz, Methanol-d₄): δ 9.40 (s, 1H), 8.33 (s, 1H), 8.22 (d, J=8.4 Hz, 1H), 7.81-7.71 (m, 2H), 7.55-7.36 (m, 2H), 5.24-5.09 (m, 1H), 4.62-4.39 (m, 4H), 3.76-3.62 (m, 2H), 3.33-3.19 (m, 2H), 3.13-2.99 (m, 1H), 2.95-2.73 (m, 2H), 2.60-2.38 (m, 1H), 2.24-1.95 (m, 5H).

### 7.16. Example 16: Selectivity of Compound A, Compound B, Compound C, and Compound D

DF15 multiple myeloma cells stably expressing ePL-tagged Aiolos, Ikaros, or GSPT1, and MDS-L cells stably expressing ePL-tagged CK1a were generated via lentiviral infection with pLOC-ePL-Aiolos (or Ikaros, GSPT1, or CK1a). DF15 multiple myeloma cells expressing Ikaros, Aiolos, and GSPT1 fused to an ePL tag (DiscoverX) and MDS-L cells expressing CK1a fused to ePL tag were dispensed into a 384-well plate (Corning no. 3570) prespotted with compounds (Compound A, Compound B, Compound C, and Compound D). Compounds were dispensed by an acoustic dispenser (ATS acoustic transfer system from EDC Biosystems) into a 384-well plate in a 10-point dose response curve using 3-fold dilutions starting at 10 μM and going down to 0.0005 M. Then, 25 μL of media (RPMI-1640+10% heat inactivated FBS+25 mM Hepes+1 mM Na pyruvate+1× NEAA+1×Pen Strep Glutamine) containing 5000 of DF15 or MSD-L cells was dispensed per well. Assay plates were incubated at 37° C. with 5% CO₂ for 4 hours except 20 hours for GSPT1. After incubation, 25 μL of the InCELL Hunter detection reagent working solution (DiscoverX, catalogue no. 96-0002, Fremont, Calif.) was added to each well and incubated at room temperature for 60 min protected from light. After 60 min, luminescence was read on an Envision or PHERAstar luminescence reader.

For Helios, a stable Jurkat cell line was engineered using CRISPR/Cas9 to insert an in-frame HiBit tag into the carboxy-terminal reading frame of the IKZF2 gene. Test compounds were transferred to 1536 well plates using an acoustic dispenser, and Jurkat/Helios/HiBit cells in DMEM/10% FCS were plated at 10,000 cells/well in a final volume of 5 ul. Cells were incubated at 37C, 95% RH for 18 hr. Luciferase activity was measured by adding 2 ul/well of Nano-Glo reagent (Promega), incubating at RT for 30 min, and reading luminescence on a microtiter plate reader.

To determine the EC50 value of a compound for the degradation of a given substrate (concentration of compound that achieves half the maximum degradation observed), a four-parameter logistic model (sigmoidal dose—response model) (FIT=(A+{(B−A)/1+[(C/x)D]})) where C is the inflection point (EC50), D is the correlation coefficient, and A and B are the low and high limits of the fit, respectively) was used. All substrate degradation curves were processed and evaluated using ActivityBase (IDBS), a data analysis software package. Ymin is minimum percent protein remaining.

The results are shown in Tables 2-5.

TABLE 2
Compound A

| Degron | Ymin (%) | EC50 (μM) |
|---|---|---|
| Q1F | NA | NA |
| Helios | 1.5 | 0.0018 |
| Aiolos | 2.7 | 0.0064 |
| Ikaros | 3.2 | 0.022 |
| GSPTI | 91 | 0.039 |
| CKla | 50 | 0.19 |

TABLE 3
Compound B

| Degron | Ymin (%) | EC50 (μM) |
|---|---|---|
| Q1F (from Table 1) | 1.7 | 0.0048 |
| Helios | 65 | >10 |
| Aiolos | 93 | >10 |
| Ikaros | 91 | >10 |
| GSPT1 | 72 | 0.054 |
| CK1a | 82 | >10 |

TABLE 4
Compound C

| Degron | Ymin (%) | EC50 (μM) |
|---|---|---|
| Q1F (from Table 1) | 12 | 0.023 |
| Helios | 86 | >10 |
| Aiolos | 83 | >10 |
| Ikaros | 92 | >10 |
| GSPTI | 89 | >10 |
| CK1a | 98 | >10 |

TABLE 5
Compound D

| Degron | Ymin (%) | EC50 (μM) |
|---|---|---|
| Q1F (from Table 1) | 4.2 | 0.0007 |
| Helios | 24 | 0.46 |
| Aiolos | 87 | >10 |
| Ikaros | 93 | >10 |
| GSPT1 | 96 | >10 |
| CK1a | 81 | >10 |

As shown in Table 2, Compound A degraded Helios, Aiolos, and Ikaros, with an EC50 of 0.022 μM or lower. Compound A also degraded CK1a to a significant extent. In contrast, Compound B degraded the Q1F degron with an EC50 of less than 5 nM in the Jurkat assay described in Example 5. While Compound B showed some degradation activity against Helios, the EC50 was >10 μM, which is more than 3 orders of magnitude higher than the EC50 for the Q1F degron (Table 3). Compound B also showed some degradation activity against GSPT1 (Table 3). Compound C was highly selective for the Q1F degron in the Jurkat assay described in Example 5, with an EC50 of less than 25 nM (Table 4). Compound D was also selective for the Q1F degron in the Jurkat assay described in Example 5, with an EC50 of less than 1 nM (Table 5), although it also showed some degradation of Helios, with an EC50 of 0.46 μM (Table 5).

### EQUIVALENTS

The present disclosure is not limited in scope by the specific embodiments described herein. Indeed, various modifications of the subject matter provided herein, in addition to those described, will become apparent to those skilled in the art from the foregoing description. Such modifications are intended to fall within the scope of the appended claims.

Various publications, patents and patent applications are cited herein, the disclosures of which are incorporated by reference in their entireties.

TABLE of
Certain Sequences

| SEQ | | |
|---|---|---|
| ID | | |
| NO | Description | Sequence |
| 1 | XIF G-motif consensus 1 | FCX₁X₂CGX₃X₄ |
| 2 | XIF G-motif consensus 2 | FCX₁X₂CGX₃X₄X₅ |
| 3 | IKZF1 ZNF2 Q1F G-motif | FCNQCGAS |
| 4 | IKZF1 ZNF2 Q1F | GERPFFCNQCGASFTQKGNLLRHIKLHS |
| 5 | IKZF1 ZNF2_ZNF3 Q1F | GERPFFCX₁X₂C GX₃X₄X₅TQKGNL LRHIKLHSGE |
| | consensus | KPFKCHLCNY ACRRRDALTG HLRTHS |
| 66 | IKZF2 ZNF2_ZNF3 Q1F | GERPFFCX₁X₂C GX₃X₄X₅TQKGNL LRHIKLHSGE |
| | consensus | KPFKCPFCSY ACRRRDALTG HLRTHS |
| 67 | IKZF3 ZNF2_ZNF3 Q1F | GERPFFCX₁X₂C GX₃X₄X₅TQKGNL LRHIKLHSGE |
| | consensus | KPFKCHLCNY ACQRRDALTG HLRTHS |
| 68 | IKZF4 ZNF2_ZNF3 Q1F | GERPFFCX₁X₂C GX₃X₄X₅TQKGNL LRHIKLHSGE |
| | consensus | KPFKCPFCNY ACRRRDALTG HLRTHS |
| 6 | IKZF1 ZNF2_ZNF3 | GERPFQCNQC GASFTQKGNL LRHIKLHSGE KPFKCHLCNY |
| | | ACRRRDALTG HLRTHS |
| 63 | IKZF2 ZNF2_ZNF3 | GERPFHCNQC GASFTQKGNL LRHIKLHSGE KPFKCPFCSY |
| | | ACRRRDALTG HLRTHS |
| 64 | IKZF3 ZNF2_ZNF3 | GERPFQCNQC GASFTQKGNL LRHIKLHTGE KPFKCHLCNY |
| | | ACQRRDALTG HLRTHS |
| 65 | IKZF4 ZNF2_ZNF3 | GERPFHCNQC GASFTQKGNL LRHIKLHSGE KPFKCPFCNY |
| | | ACRRRDALTG HLRTHS |
| 7 | IKZF1 ZNF2_ZNF3 Q1F | GERPFFCNQC GASFTQKGNL LRHIKLHSGE KPFKCHLCNY |
| | | ACRRRDALTG HLRTHS |
| 69 | IKZF2 ZNF2_ZNF3 Q1F | GERPFFCNQC GASFTQKGNL LRHIKLHSGE KPFKCPFCSY |
| | | ACRRRDALTG HLRTHS |
| 70 | IKZF3 ZNF2_ZNF3 Q1F | GERPFFCNQC GASFTQKGNL LRHIKLHTGE KPFKCHLCNY |
| | | ACQRRDALTG HLRTHS |
| 71 | IKZF4 ZNF2_ZNF3 Q1F | GERPFFCNQC GASFTQKGNL LRHIKLHSGE KPFKCPFCNY |
| | | ACRRRDALTG HLRTHS |
| 8 | IKZF1 ZNF1_ZNF2 | ggcattcgac ttcctaacgg aaaactaaag tgtgatatct |
| | nucleotide sequence | gtgggatcat ttgcatcggg cccaatgtgc tcatggttca |
| | | caaaagaagc cacactGGAG AACGGCCCTT CCAGTGCAAT |
| | | CAGTGCGGGG CCTCATTCAC CCAGAAGGGC AACCTGCTCC |
| | | GGCACATCAA GCTGCATTCC |
| 9 | IKZF1 ZNF1_ZNF2 | GIRLPNGKLK CDICGIICIG PNVLMVHKRS HTGERPFQCN |
| | | QCGASFTQKG NLLRHIKLHS |
| 10 | IKZF1 ZNF1_ZNF2_ZNF3 | ggcattcgac ttcctaacgg aaaactaaag tgtgatatct |
| | nucleotide sequence | gtgggatcat ttgcatcggg cccaatgtgc tcatggttca |
| | | caaaagaagc cacactGGAG AACGGCCCTT CCAGTGCAAT |
| | | CAGTGCGGGG CCTCATTCAC CCAGAAGGGC AACCTGCTCC |
| | | GGCACATCAA GCTGCATTCC ggggagaagc ccttcaaatg |
| | | ccacctctgc aactacgcct gccgccggag ggacgccctc |
| | | actggccacc tgaggacgca ctcc |
| 11 | IKZF1 ZNF1_ZNF2_ZNF3 | GIRLPNGKLK CDICGIICIG PNVLMVHKRS HTGERPFQCN |
| | | QCGASFTQKG NLLRHIKLHS GEKPFKCHLC NYACRRRDAL |
| | | TGHLRTHS |
| 12 | IKZF1 ZNF2_ZNF3 | GGAGAACGGC CCTTCCAGTG CAATCAGTGC GGGGCCTCAT |
| | nucleotide sequence | TCACCCAGAA GGGCAACCTG CTCCGGCACA TCAAGCTGCA |
| | | TTCCggggag aagcccttca aatgccacct ctgcaactac |
| | | gcctgccgcc ggagggacgc cctcactggc cacctgagga |
| | | cgcactcc |
| 13 | IKZF1 ZNF2_ZNF3 | GERPFQCNQC GASFTQKGNL LRHIKLHSGE KPFKCHLCNY |
| | | ACRRRDALTG HLRTHS |
| 14 | IKZF1 ZNF2 nucleotide | GGAGAACGGC CCTTCCAGTG CAATCAGTGC GGGGCCTCAT |
| | sequence | TCACCCAGAA GGGCAACCTG CTCCGGCACA TCAAGCTGCA |
| | | TTCC |
| 15 | IKZF1 ZNF2 | GERPFQCNQC GASFTQKGNL LRHIKLHS |
| 60 | IKZF2 ZNF2 | GERPFHCNQC GASFTQKGNLLRHIKLHS |
| 61 | IKZF3 ZNF2 | GERPFQCNQC GASFTQKGNLLRHIKLHT |
| 62 | IKZF4 ZNF2 | GERPFHCNQCGASFTQKGNLLRHIKLHS |
| 16 | IKZF1 ZNF2_ZNF3 G6N | GGAGAACGGC CCTTCCAGTG CAATCAGTGC aacGCCTCAT |
| | nucleotide sequence | TCACCCAGAA GGGCAACCTG CTCCGGCACA TCAAGCTGCA |
| | | TTCCggggag aagcccttca aatgccacct ctgcaactac |
| | | gcctgccgcc ggagggacgc cctcactggc cacctgagga |
| | | cgcactcc |
| 17 | IKZF1 ZNF2_ZNF3 G6N | GERPFQCNQC NASFTQKGNL LRHIKLHSGE KPFKCHLCNY |
| | | ACRRRDALTG HLRTHS |
| 18 | IKZF1 ZNF2_ZNF3 Q1F | ggagaacggc ccttcttctg caatcagtgc ggggcctcat |
| | nucleotide sequence | tcacccagaa gggcaacctg ctccggcaca tcaagctgca |
| | | ttccggggag aagcccttca aatgccacct ctgcaactac |
| | | gcctgccgcc ggagggacgc cctcactggc cacctgagga |
| | | cgcactcc |
| 19 | IKZF1 ZNF2_ZNF3 Q1F | GERPFFCNQC GASFTQKGNL LRHIKLHSGE KPFKCHLCNY |
| | | ACRRRDALTG HLRTHS |
| 59 | IKZF1 ZNF2_ZNF3 Q1F/G6N | GERPFFCNQC NASFTQKGNL LRHIKLHSGE KPFKCHLCNY |
| | | ACRRRDALTG HLRTHS |
| 20 | IKZF1 ZNF1 (amino acids | LKCDICGIICIGPNVLMVHKRSH |
| | 117-139) | |
| 21 | IKZF1 ZNF2 (amino acids | FQCNQCGASFTQKGNLLRHIKLH |
| | 145-167) | |
| 22 | IKZF1 ZNF3 (amino acids | FKCHLCNYACRRRDALTGHLRTH |
| | 173-195) | |
| 23 | IKZF1 ZNF4 (amino acids | HKCGYCGRSYKQRSSLEEHKERCH |
| | 201-224) | |
| 24 | IKZF1 ZNF5 (amino acids | YKCEHCRVLFLDHVMYTIHMGCH |
| | 462-484) | |
| 25 | IKZF1 ZNF6 (amino acids | FECNMCGYHSQDRYEFSSHITRGEH |
| | 490-514) | |
| 26 | IKZF2 ZNF1 (amino acids | LKCDVCGMVCIGPNVLMVHKRSH |
| | 112-134) | |
| 27 | IKZF2 ZNF2 (amino acids | FHCNQCGASFTQKGNLLRHIKLH |
| | 140-162) | |
| 28 | IKZF2 ZNF3 (amino acids | FKCPFCSYACRRRDALTGHLRTH |
| | 168-190) | |
| 29 | IKZF2 ZNF4 (amino acids | HKCNYCGRSYKQRSSLEEHKERCH |
| | 196-219) | |
| 30 | IKZF2 ZNF5 (amino acids | FKCEHCRVLFLDHVMYTIHMGCH |
| | 471-493) | |
| 58 | IKZF2 ZNF6 (amino acids | LECNICGYRSQDRYEFSSHIVRGEH |
| | 499-523) | |
| 31 | IKZF3 ZNF1 (amino acids | MNCDVCGLSCISFNVLMVHKRSH |
| | 118-140) | |
| 32 | IKZF3 ZNF2 (amino acids | FQCNQCGASFTQKGNLLRHIKLH |
| | 146-168) | |
| 33 | IKZF3 ZNF3 (amino acids | FKCHLCNYACQRRDALTGHLRTH |
| | 174-196) | |
| 34 | IKZF3 ZNF4 (amino acids | YKCEFCGRSYKQRSSLEEHKERC |
| | 202-224) | |
| 35 | IKZF3 ZNF5 (amino acids | YRCDHCRVLFLDYVMFTIHMGCH |
| | 452-474) | |
| 36 | IKZF3 ZNF6 (amino acids | FECNMCGYRSHDRYEFSSHIARGEH |
| | 480-504) | |
| 37 | IKZF4 ZNF1 (amino acids | LKCDVCGMVCIGPNVLMVHKRSH |
| | 159-181) | |
| 38 | IKZF4 ZNF2 (amino acids | FHCNQCGASFTQKGNLLRHIKLH |
| | 187-209) | |
| 39 | IKZF4 ZNF3 (amino acids | FKCPFCNYACRRRDALTGHLRTH |
| | 215-237) | |
| 40 | IKZF4 ZNF4 (amino acids | YKCNYCGRSYKQQSTLEEHKERCH |
| | 248-271) | |
| 41 | IKZF4 ZNF5 (amino acids | FKCEHCRILFLDHVMFTIHMGCH |
| | 530-552) | |
| 42 | IKZF4 ZNF6 (amino acids | FECNICGYHSQDRYEFSSHIVRGEH |
| | 558-582) | |
| 43 | IKZF5 ZNF1 (amino acids | LKCRYCNYAS KGTARLIEHIRIH |
| | 82-104) | |
| 44 | IKZF5 ZNF2 (amino acids | HRCHLCPFASAYERHLEAHMRSH |
| | 110-132) | |
| 45 | IKZF5 ZNF3 (amino acids | YKCELCSFRCSDRSNLSHHRRRKH |
| | 138-161) | |
| 46 | IKZF5 ZNF4 (amino acids | HHCQHCDMYFADNILYTIHMGCH |
| | 364-386) | |
| 47 | IKZF5 ZNF5 (amino acids | FQCNICGCKCKNKYDFACHFARGQH |
| | 392-416) | |
| 48 | Human Ikaros amino acid | MDADEGQDMS QVSGKESPPV SDTPDEGDEP MPIPEDLSTT |
| | sequence | SGGQQSSKSD RVVASNVKVE TQSDEENGRA CEMNGEECAE |
| | | DLRMLDASGE KMNGSHRDQG SSALSGVGGI RLPNGKLKCD |
| | | ICGIICIGPN VLMVHKRSHT GERPFQCNQC GASFTQKGNL |
| | | LRHIKLHSGE KPFKCHLCNY ACRRRDALTG HLRTHSVGKP |
| | | HKCGYCGRSY KQRSSLEEHK ERCHNYLESM GLPGTLYPVI |
| | | KEETNHSEMA EDLCKIGSER SLVLDRLASN VAKRKSSMPQ |
| | | KFLGDKGLSD TPYDSSASYE KENEMMKSHV MDQAINNAIN |
| | | YLGAESLRPL VQTPPGGSEV VPVISPMYQL HKPLAEGTPR |
| | | SNHSAQDSAV ENLLLLSKAK LVPSEREASP SNSCQDSTDT |
| | | ESNNEEQRSG LIYLTNHIAP HARNGLSLKE EHRAYDLLRA |
| | | ASENSQDALR VVSTSGEQMK VYKCEHCRVL FLDHVMYTIH |
| | | MGCHGFRDPF ECNMCGYHSQ DRYEFSSHIT RGEHRFHMS |
| 49 | Human Aiolos amino acid | MEDIQTNAEL KSTQEQSVPA ESAAVLNDYS LTKSHEMENV |
| | sequence | DSGEGPANED EDIGDDSMKV KDEYSERDEN VLKSEPMGNA |
| | | EEPEIPYSYS REYNEYENIK LERHVVSFDS SRPTSGKMNC |
| | | DVCGLSCISF NVLMVHKRSH TGERPFQCNQ CGASFTQKGN |
| | | LLRHIKLHTG EKPFKCHLCN YACQRRDALT GHLRTHSVEK |
| | | PYKCEFCGRS YKQRSSLEEH KERCRTFLQS TDPGDTASAE |
| | | ARHIKAEMGS ERALVLDRLA SNVAKRKSSM PQKFIGEKRH |
| | | CFDVNYNSSY MYEKESELIQ TRMMDQAINN AISYLGAEAL |
| | | RPLVQTPPAP TSEMVPVISS MYPIALTRAE MSNGAPQELE |
| | | KKSIHLPEKS VPSERGLSPN NSGHDSTDTD SNHEERQNHI |
| | | YQQNHMVLSR ARNGMPLLKE VPRSYELLKP PPICPRDSVK |
| | | VINKEGEVMD VYRCDHCRVL FLDYVMFTIH MGCHGFRDPF |
| | | ECNMCGYRSH DRYEFSSHIA RGEHRALLK |
| 50 | Human Helios amino acid | METEAIDGYI TCDNELSPER EHSNMAIDLT SSTPNGQHAS |
| | sequence | PSHMTSTNSV KLEMQSDEEC DRKPLSREDE IRGHDEGSSL |
| | | EEPLIESSEV ADNRKVQELQ GEGGIRLPNG KLKCDVCGMV |
| | | CIGPNVLMVH KRSHTGERPF HCNQCGASFT QKGNLLRHIK |
| | | LHSGEKPFKC PFCSYACRRR DALTGHLRTH SVGKPHKCNY |
| | | CGRSYKQRSS LEEHKERCHN YLQNVSMEAA GQVMSHHVPP |
| | | MEDCKEQEPI MDNNISLVPF ERPAVIEKLT GNMGKRKSST |
| | | PQKFVGEKLM RFSYPDIHFD MNLTYEKEAE LMQSHMMDQA |
| | | INNAITYLGA EALHPLMQHP PSTIAEVAPV ISSAYSQVYH |
| | | PNRIERPISR ETADSHENNM DGPISLIRPK SRPQEREASP |
| | | SNSCLDSTDS ESSHDDHQSY QGHPALNPKR KQSPAYMKED |
| | | VKALDTTKAP KGSLKDIYKV FNGEGEQIRA FKCEHCRVLF |
| | | LDHVMYTIHM GCHGYRDPLE CNICGYRSQD RYEFSSHIVR |
| | | GEHTFH |
| 51 | Human Eos amino acid | MHTPPALPRR FQGGGRVRTP GSHRQGKDNL ERDPSGGCVP |
| | sequence | DFLPQAQDSN HFIMESLFCE SSGDSSLEKE FLGAPVGPSV |
| | | STPNSQHSSP SRSLSANSIK VEMYSDEESS RLLGPDERLL |
| | | EKDDSVIVED SLSEPLGYCD GSGPEPHSPG GIRLPNGKLK |
| | | CDVCGMVCIG PNVLMVHKRS HTGERPFHCN QCGASFTQKG |
| | | NLLRHIKLHS GEKPFKCPFC NYACRRRDAL TGHLRTHSVS |
| | | SPTVGKPYKC NYCGRSYKQQ STLEEHKERC HNYLQSLSTE |
| | | AQALAGQPGD EIRDLEMVPD SMLHSSSERP TFIDRLANSL |
| | | TKRKRSTPQK FVGEKQMRFS LSDLPYDVNS GGYEKDVELV |
| | | AHHSLEPGFG SSLAFVGAEH LRPLRLPPTN CISELTPVIS |
| | | SVYTQMQPLP GRLELPGSRE AGEGPEDLAD GGPLLYRPRG |
| | | PLTDPGASPS NGCQDSTDTE SNHEDRVAGV VSLPQGPPPQ |
| | | PPPTIVVGRH SPAYAKEDPK PQEGLLRGTP GPSKEVLRVV |
| | | GESGEPVKAF KCEHCRILFL DHVMFTIHMG CHGFRDPFEC |
| | | NICGYHSQDR YEFSSHIVRG EHKVG |
| 52 | Human Pegasus amino acid | MGEKKPEPLD FVKDFQEYLT QQTHHVNMIS GSVSGDKEAE |
| | sequence | ALQGAGTDGD QNGLDHPSVE VSLDENSGML VDGFERTFDG |
| | | KLKCRYCNYA SKGTARLIEH IRIHTGEKPH RCHLCPFASA |
| | | YERHLEAHMR SHTGEKPYKC ELCSFRCSDR SNLSHHRRRK |
| | | HKMVPIKGTR SSLSSKKMWG VLQKKTSNLG YSRRALINLS |
| | | PPSMVVQKPD YLNDFTHEIP NIQTDSYESM AKTTPTGGLP |
| | | RDPQELMVDN PLNQLSTLAG QLSSLPPENQ NPASPDVVPC |
| | | PDEKPFMIQQ PSTQAVVSAV SASIPQSSSP TSPEPRPSHS |
| | | QRNYSPVAGP SSEPSAHTST PSIGNSQPST PAPALPVQDP |
| | | QLLHHCQHCD MYFADNILYT IHMGCHGYEN PFQCNICGCK |
| | | CKNKYDFACH FARGQHNQH |
| 53 | CAR CD19 CD28TM 4IBB | DIQMTQTT SSLSASLGDR VTISCRASQD ISKYLNWYQQ |
| | CD3z | KPDGTVKLLI YHTSRLHSGV PSRFSGSGSG TDYSLTISNL |
| | | EQEDIATYFC QQGNTLPYTF GGGTKLEITG STSGSGKPGS |
| | | GEGSTKGEVK LQESGPGLVA PSQSLSVTCT VSGVSLPDYG |
| | | VSWIRQPPRK GLEWLGVIWG SETTYYNSAL KSRLTIIKDN |
| | | SKSQVFLKMN SLQTDDTAIY YCAKHYYYGG SYAMDYWGQG |
| | | TSVTVSSESK YGPPCPPCPM FWVLVVVGGV LACYSLLVTV |
| | | AFIIFWVKRG RKKLLYIFKQ PFMRPVQTTQ EEDGCSCRFP |
| | | EEEEGGCELR VKFSRSADAP AYQQGQNQLY NELNLGRREE |
| | | YDVLDKRRGR DPEMGGKPRR KNPQEGLYNE LQKDKMAEAY |
| | | SEIGMKGERR RGKGHDGLYQ GLSTATKDTY DALHMQALPP |
| | | RGGGEGRGSL LTCGDVEENP G |
| 54 | CD28TM 41BB CD3z ZNF2 | DIQMTQTT SSLSASLGDR VTISCRASQD ISKYLNWYQQ |
| | | KPDGTVKLLI YHTSRLHSGV PSRFSGSGSG TDYSLTISNL |
| | | EQEDIATYFC QQGNTLPYTF GGGTKLEITG STSGSGKPGS |
| | | GEGSTKGEVK LQESGPGLVA PSQSLSVTCT VSGVSLPDYG |
| | | VSWIRQPPRK GLEWLGVIWG SETTYYNSAL KSRLTIIKDN |
| | | SKSQVFLKMN SLQTDDTAIY YCAKHYYYGG SYAMDYWGQG |
| | | TSVTVSSESK YGPPCPPCPM FWVLVVVGGV LACYSLLVTV |
| | | AFIIFWVKRG RKKLLYIFKQ PFMRPVQTTQ EEDGCSCRFP |
| | | EEEEGGCELR VKFSRSADAP AYQQGQNQLY NELNLGRREE |
| | | YDVLDKRRGR DPEMGGKPRR KNPQEGLYNE LQKDKMAEAY |
| | | SEIGMKGERR RGKGHDGLYQ GLSTATKDTY DALHMQALPP |
| | | RTGERPFQCN QCGASFTQKG NLLRHIKLHS GGGEGRGSLL |
| | | TCGDVEENPG |
| 55 | CD28TM 41BB CD3z ZNF1_2 | DIQMTQTT SSLSASLGDR VTISCRASQD ISKYLNWYQQ |
| | | KPDGTVKLLI YHTSRLHSGV PSRFSGSGSG TDYSLTISNL |
| | | EQEDIATYFC QQGNTLPYTF GGGTKLEITG STSGSGKPGS |
| | | GEGSTKGEVK LQESGPGLVA PSQSLSVTCT VSGVSLPDYG |
| | | VSWIRQPPRK GLEWLGVIWG SETTYYNSAL KSRLTIIKDN |
| | | SKSQVFLKMN SLQTDDTAIY YCAKHYYYGG SYAMDYWGQG |
| | | TSVTVSSESK YGPPCPPCPM FWVLVVVGGV LACYSLLVTV |
| | | AFIIFWVKRG RKKLLYIFKQ PFMRPVQTTQ EEDGCSCRFP |
| | | EEEEGGCELR VKFSRSADAP AYQQGQNQLY NELNLGRREE |
| | | YDVLDKRRGR DPEMGGKPRR KNPQEGLYNE LQKDKMAEAY |
| | | SEIGMKGERR RGKGHDGLYQ GLSTATKDTY DALHMQALPP |
| | | RGIRLPNGKL KCDICGIICI GPNVLMVHKR SHTGERPFQC |
| | | NQCGASFTQK GNLLRHIKLH SGGGEGRGSL LTCGDVEENP |
| | | G |
| 56 | CD28TM 41BB CD3z ZNF2_3 | DIQMTQTT SSLSASLGDR VTISCRASQD ISKYLNWYQQ |
| | | KPDGTVKLLI YHTSRLHSGV PSRFSGSGSG TDYSLTISNL |
| | | EQEDIATYFC QQGNTLPYTF GGGTKLEITG STSGSGKPGS |
| | | GEGSTKGEVK LQESGPGLVA PSQSLSVTCT VSGVSLPDYG |
| | | VSWIRQPPRK GLEWLGVIWG SETTYYNSAL KSRLTIIKDN |
| | | SKSQVFLKMN SLQTDDTAIY YCAKHYYYGG SYAMDYWGQG |
| | | TSVTVSSESK YGPPCPPCPM FWVLVVVGGV LACYSLLVTV |
| | | AFIIFWVKRG RKKLLYIFKQ PFMRPVQTTQ EEDGCSCRFP |
| | | EEEEGGCELR VKFSRSADAP AYQQGQNQLY NELNLGRREE |
| | | YDVLDKRRGR DPEMGGKPRR KNPQEGLYNE LQKDKMAEAY |
| | | SEIGMKGERR RGKGHDGLYQ GLSTATKDTY DALHMQALPP |
| | | RGERPFQCNQ CGASFTQKGN LLRHIKLHSG EKPFKCHLCN |
| | | YACRRRDALT GHLRTHSGGG EGRGSLLTCG DVEENPG |
| 57 | CD28TM41BB CD3z | DIQMTQTT SSLSASLGDR VTISCRASQD ISKYLNWYQQ |
| | ZNF1_2_3 | KPDGTVKLLI YHTSRLHSGV PSRFSGSGSG TDYSLTISNL |
| | | EQEDIATYFC QQGNTLPYTF GGGTKLEITG STSGSGKPGS |
| | | GEGSTKGEVK LQESGPGLVA PSQSLSVTCT VSGVSLPDYG |
| | | VSWIRQPPRK GLEWLGVIWG SETTYYNSAL KSRLTIIKDN |
| | | SKSQVFLKMN SLQTDDTAIY YCAKHYYYGG SYAMDYWGQG |
| | | TSVTVSSESK YGPPCPPCPM FWVLVVVGGV LACYSLLVTV |
| | | AFIIFWVKRG RKKLLYIFKQ PFMRPVQTTQ EEDGCSCRFP |
| | | EEEEGGCELR VKFSRSADAP AYQQGQNQLY NELNLGRREE |
| | | YDVLDKRRGR DPEMGGKPRR KNPQEGLYNE LQKDKMAEAY |
| | | SEIGMKGERR RGKGHDGLYQ GLSTATKDTY DALHMQALPP |
| | | RGIRLPNGKL KCDICGIICI GPNVLMVHKR SHTGERPFQC |
| | | NQCGASFTQK GNLLRHIKLH SGEKPFKCHL CNYACRRRDA |
| | | LTGHLRTHSG GGEGRGSLLT CGDVEENPG |
| 72 | Q9UKT9_IKZF3_146_168_23 | FQCNQCGASFTQKGNLLRHIKLH |
| 73 | Q66K89_E4F1_220_242_23 | HECKLCGASFRTKGSLIRHHRRH |
| 74 | Q6ZMY9_ZN517_205_227_23 | FQCTECGKAFKQSSILLRHQLIH |
| 75 | Q6DD87_ZN787_178_200_23 | FVCPRCGRGFSQPKSLARHLRLH |
| 76 | Q6ZMY9_ZN517_396_418_23 | FECAECGKAFGRKSNLTLHQKIH |
| 77 | Q96JP5_ZFP91_400_422_23 | LQCEICGFTCRQKASLNWHMKKH |
| 78 | Q6DD87_ZN787_150_172_23 | YTCPDCGRSFTQSKSLAKHRRSH |
| 79 | Q96CK0_ZN653_528_550_23 | FTCETCGKSFKRKNHLEVHRRTH |
| 80 | Q17R98_ZN827_817_839_23 | FPCDVCGKVFGRQQTLSRHLSLH |
| 81 | Q6DD87_ZN787_317_339_23 | HICVECGEGFVQGAALRRHKKIH |
| 82 | Q6DD87_ZN787_66_88_23 | YICNECGKSFSHWSKLTRHQRTH |
| 83 | Q6ZMY9_ZN517_233_255_23 | FQCGECGKAFRQSTQLAAHHRVH |
| 84 | Q96CK0_ZN653_556_578_23 | LQCEICGYQCRQRASLNWHMKKH |
| 85 | Q9BU19_ZN692_417_439_23 | LQCEICGFTCRQKASLNWHQRKH |
| 86 | Q6ZMY9_ZN517_317_339_23 | YRCLRCGQRFIRGSSLLKHHRLH |
| 87 | Q6ZMY9_ZN517_261_283_23 | YACGECGKAFSRSSRLLQHQKFH |
| 88 | Q8IZM8_ZN654_25_47_23 | FACVICGRKFRNRGLMQKHLKNH |
| 89 | Q17R98_ZN827_374_396_23 | FQCPICGLVIKRKSYWKRHMVIH |
| 90 | Q6ZMY9_ZN517_368_390_23 | HECPVCGRPFRHNSLLLLHLRLH |
| 91 | Q8N554_ZN276_496_18_23 | YICDECGQTFKQRKHLLVHQMRH |
| 92 | Q66K89_E4F1_463_485_23 | FACAQCGKAFPKAYLLKKHQEVH |
| 93 | Q6ZMY9_ZN517_424_446_23 | FACTECGKAFRRSYTLNEHYRLH |
| 94 | Q96NG8_ZN582_339_361_23 | YECKECGKAFNQGSTLIRHQRIH |
| 95 | Q8N554_ZN276_524_546_23 | LQCEVCGFQCRQRASLKYHMTKH |
| 96 | Q6DD87_ZN787_94_116_23 | NACADCGKTFSQSSHLVQHRRIH |
| 97 | Q6DD87_ZN787_122_144_23 | YACLECGKRFSWSSNLMQHQRIH |
| 98 | Q6ZMY9_ZN517_289_311_23 | FACTECGKAFCRRFTLNEHGRIH |
| 99 | Q96NG8_ZN582_283_305_23 | YQCKECGKAFNRISHLKVHYRIH |
| 100 | Q96NG8_ZN582_395_417_23 | YQCKVCGRAFKRVSHLTVHYRIH |
| 101 | Q6ZMY9_ZN517_452_474_23 | YRCRACGRACSRLSTLIQHQKVH |
| 102 | Q9UKT9_IKZF3_118_140_23 | MNCDVCGLSCISFNVLMVHKRSH |
| 103 | Q96NG8_ZN582_367_389_23 | YECKVCGKAFRVSSQLKQHQRIH |
| 104 | Q96NG8_ZN582_311_333_23 | YACKECGKTFSHRSQLIQHQTVH |
| 105 | Q96NG8_ZN582_423_445_23 | YECKECGKAFSHCSQLIHHQVIH |
| 106 | Q96NG8_ZN582_199_221_23 | YKCKECGKAFKYGSRLIQHENIH |
| 107 | Q96NG8_ZN582_227_249_23 | YECKECGKAFNSGSNFIQHQRVH |
| 108 | Q66K89_E4F1_491_513_23 | FRCGDCGKLYKTIAHVRGHRRVH |
| 109 | Q66K89_E4F1_519_541_23 | YPCPKCGKRYKTKNAQQVHFRTH |

1. An engineered polypeptide comprising a degradation domain, wherein the degradation domain comprises the amino acid sequence FCX₁X₂CGX₃X₄ (SEQ ID NO: 1), wherein:
X₁ is selected from asparagine, aspartate, glycine, glutamine, methionine, histidine, tryptophan, isoleucine, arginine, leucine, valine, threonine, and phenylalanine,
X₂ is selected from glutamine, arginine, histidine, leucine, phenylalanine, tyrosine, tryptophan, isoleucine, valine, and methionine,
X₃ is selected from alanine, serine, cysteine, arginine, leucine, isoleucine, methionine, and glycine, and
X₄ is selected from serine, methionine, lysine, isoleucine, valine, histidine, glutamine, arginine, phenylalanine, and tryptophan.

2. The engineered polypeptide of claim 1, wherein:
X₁ is selected from asparagine, glutamine, methionine, histidine, tryptophan, isoleucine, arginine, leucine, valine, threonine, and phenylalanine,
X₂ is selected from glutamine, arginine, histidine, leucine, phenylalanine, tyrosine, tryptophan, isoleucine, and methionine,
X₃ is selected from alanine, serine, cysteine, and glycine, and
X₄ is selected from serine, methionine, histidine, glutamine, arginine, phenylalanine, and tryptophan.

3. The engineered polypeptide of claim 1, wherein the degradation domain comprises the amino acid sequence FCX₁X₂CGX₃X₄X₅ (SEQ ID NO: 2), wherein:
X₅ is selected from phenylalanine, tryptophan, methionine, arginine, histidine, leucine, tyrosine, cysteine, and glutamine.

4. (canceled)

5. The engineered polypeptide of claim 3, wherein X₅ is selected from phenylalanine, tryptophan, methionine, leucine, tyrosine, and glutamine.

6.-11. (canceled)

12. The engineered polypeptide of claim 1, wherein the degradation domain comprises the amino acid sequence FCNQCGAS (SEQ ID NO: 3).

13. (canceled)

14. The engineered polypeptide of claim 1, wherein the degradation domain comprises two zinc finger domains.

15. The engineered polypeptide of claim 1, wherein at least one or each zinc finger domain independently comprises an amino acid sequence that is at least 85%, at least 90%, or at least 95% identical to an amino acid sequence selected from SEQ ID NOs: 20-47 and 58.

16. The engineered polypeptide of claim 1, wherein the degradation domain comprises
(a) an amino acid sequence that is at least 85%, at least 90%, or at least 95% identical to zinc finger 2 (ZNF2) of human Ikaros, Helios, Aiolos, or Eos;
(b) an amino acid sequence that is at least 85%, at least 90%, or at least 95% identical to ZNF1 or ZNF3 of human Ikaros, Helios, Aiolos, or Eos; or
(c) both (a) and (b).

17.-18. (canceled)

19. The engineered polypeptide of claim 1, wherein the degradation domain comprises an amino acid sequence that is at least 85%, at least 90%, or at least 95% identical to an amino acid sequence selected from SEQ ID NOs: 21, 15, 6, 27, 60, 63, 32, 61, 64, 62, 65, and 38.

20.-21. (canceled)

22. The engineered polypeptide of claim 1, wherein the degradation domain comprises an amino acid sequence selected from:
(SEQ ID NO: 5)
GERPFFCX₁X₂CGX₃X₄X₅TQKGNLLRHIKLHSGEKPFKCHLCNYACR
RRDALTGHLRTHS;
(SEQ ID NO: 66)
GERPFFCX₁X₂CGX₃X₄X₅TQKGNLLRHIKLHSGEKPFKCPFCSYACR
RRDALTGHLRTHS;
(SEQ ID NO: 67)
GERPFFCX₁X₂CGX₃X₄X₅TQKGNLLRHIKLHTGEKPFKCHLCNYACQ
RRDALTGHLRTHS;
(SEQ ID NO: 68)
GERPFFCX₁X₂CGX₃X₄X₅TQKGNLLRHIKLHSGEKPFKCPFCNYACR
RRDALTGHLRTHS;
(SEQ ID NO: 7)
GERPFFCNQCGASFTQKGNLLRHIKLHSGEKPFKCHLCNYACRRRDAL
TGHLRTHS;
(SEQ ID NO: 69)
GERPFFCNQCGASFTQKGNLLRHIKLHSGEKPFKCPFCSYACRRRDAL
TGHLRTHS;
(SEQ ID NO: 70)
GERPFFCNQCGASFTQKGNLLRHIKLHTGEKPFKCHLCNYACORRDAL
TGHLRTHS;
and
(SEQ ID NO: 71)
GERPFFCNQCGASFTQKGNLLRHIKLHSGEKPFKCPFCNYACRRRDAL
TGHLRTHS.

23. (canceled)

24. The engineered polypeptide of claim 1, wherein the engineered polypeptide is ubiquitinated and degraded in the presence of a degradation agent, wherein the degradation agent is a small molecule that binds to the degradation domain.

25.-26. (canceled)

27. The engineered polypeptide of claim 24, wherein the degradation agent mediates a complex comprising the degradation domain, degradation agent, and cereblon.

28. (canceled)

29. The engineered polypeptide of claim 24, wherein the engineered polypeptide is ubiquitinated and/or degraded in a cell in the presence of the degradation agent.

30.-31. (canceled)

32. The engineered polypeptide of claim 1, wherein the engineered polypeptide is substantially cytoplasmic or nuclear in a cell, or wherein the engineered polypeptide comprises a transmembrane domain.

33.-35. (canceled)

36. The engineered polypeptide of claim 1, wherein the engineered polypeptide comprises an endogenous protein, the degradation domain is fused to, or located within, the endogenous protein, and the endogenous protein is PRDM1, TGFBR2, CASP8, CBLB, CD5, CISH, CGKA, DGKz, MAP4K1, ARID2, BACH2, CHX37, KLF2, KLF3, KLF6, MAF, SIGLEC9, TOX, ZBTB32, PTPN2, AKT1, PIK3CD, MT1E, MT2A, CSK, ITK, PAG1, PDCD4, ZC3H12A, DNMT1, DNMT3A, PRBM1, STK4, TET2, BNIP3, FAS, CBL, BGAT5, RNF128, STK17B, TRIB1, TXNIP, UBASH3A, BATF, FLI1, IKZF1, IKZF2, IRF4, NFATC1, NR4A1, MAP2K1, MAP2K2, MAP4K4, PPARGC1A, RELB, TMEM173, USP10, MT1A, PP2A family members, RASA2, NR4A2, NR4A3, AHR, CD70, LHALS1, SOCS1, SOCS2, SOCS3, TAZ, USP21, or YAP1.

37. (canceled)

38. The engineered polypeptide of claim 1, wherein the engineered polypeptide comprises a transmembrane domain, an extracellular domain, an intracellular domain, or any combination thereof.

39. The engineered polypeptide of claim 38, wherein the transmembrane domain is the transmembrane domain of a protein selected from the alpha chain of the T-cell receptor, the beta chain of the T-cell receptor, the zeta chain of the T-cell receptor, CD28, CD3s, CD45, CD4, CD5, CD8, CD9, CD16, CD22, CD33, CD37, CD64, CD80, CD86, CD134, CD137, CD154, ICOS, TIM3, LAB3, TIGIT, PD1, or CTLA4.

40. (canceled)

41. The engineered polypeptide of claim 38, wherein the extracellular domain comprises a ligand, a ligand-binding domain, or an antigen-binding domain.

42.-43. (canceled)

44. The engineered polypeptide of claim 41, wherein the antigen-binding domain binds an antigen selected from 4-1BB, 5T4, 8H9, B7-H6, adenocarcinoma antigen, a-fetoprotein, B Cell Maturation Antigen (BCMA), BAFFR, B-lymphoma cell, C242 antigen, CA9, carcinoembryonic antigen, CA-125, carbonic anhydrase 9 (CA-IX), CCR4, CD3, CD4, CD19, CD20, CD22, CD23 (IgE receptor), CD28, CD30 (T FRSF8), CD33, CD38, CD40, CD44v6, CD44v7/8, CD51, CD52, CD56, CD70 CD74, CD80, CD123, CD152, CD171, CD200, CD221, CE7, CEA, C-MET, CLAUDIN6, CLAUDIN18.3, CNT0888, CTLA-4, DRS, EpCAM, ErbB2, ErbB3/4, EGFR, EGFRγIII, EphA2, EGP2, EGP40, FAP, Fetal AchR, fibronectin extra domain-B, folate receptor-a, folate receptor 1, G250/CAIX, GD2, GD3, glycoprotein 75, GP MB, HER2/neu, HGF, HLA-AI MAGE A1, HLA-A2 NY-ESO-1, HMW-MAA, human scatter factor receptor kinase, IGF-1 receptor, IGF-I, IgG1, IL-6, IL-13, IL-13 receptor a2, IL-11 receptor a, insulin-like growth factor I receptor, integrin a5I31, integrin avI33, Kappa light chain, L1-CAM, Lambda light chain, Lewis Y, mesothelin, MORAb-009, MS4A1, MUC1, MUC1 6, mucin CanAg, NCAM, N-glycolylneuraminic acid, NKG2D ligands, NPC-IC, PDGF-R a, PDL192, phosphatidylserine, prostate-specific cancer antigen (PSCA), prostatic carcinoma cells, PSMA, PSC1, RANKL, RON, ROR1, SCH 900105, SDC1, SLAMF7, sp17, TAG72, tenascin C, TGF (32, TGF-β, TL1A, TRAIL-R1, TRAIL-R2, tumor antigen CTAA16.88, UPK1B, VEGF-A, VEGF receptors, VEGFR-1, VEGFR2, TEM1, TEM8, and/or vimentin.

45. The engineered polypeptide of claim 44, wherein the antigen-binding domain comprises an antibody heavy chain variable region and an antibody light chain variable region, a scFv, or a single-domain antibody antigen-binding domain.

46.-47. (canceled)

48. The engineered polypeptide of claim 38, wherein the intracellular domain comprises at least one co-stimulatory domain, at least one signaling domain, or both.

49. (canceled)

50. The engineered polypeptide of claim 48, wherein at least one co-stimulatory domain is a co-stimulatory domain from a receptor protein selected from 4-1BB (CD137), CD28, OX40, an activating K cell receptor, BTLA, a Toll ligand receptor, CD2, CD7, CD27, CD30, CD40, CD5, ICAM-L LFA-1 (CD11a/CD18), B7-H3, CD5, ICAM-1, ICOS (CD278), RANK, GITR, BAFFR, LIGHT, HVEM (LIGHTR), KIRDS2, SLAMF7, Kp80 (KLRF1), Kp44, Kp30, Kp46, CD 19, CD4, CD8a, CD8p, IL2Rp, IL2Ry, IL7Ra, ITGA4, VLA1, CD49a, ITGA4, IA4, CD49D, ITGA6, VLA-6, CD49f, ITGAD, CD1 1d, ITGAE, CD103, ITGAL, CD1 1a, LFA-1, ITGAM, CD1 1b, ITGAX, CD1 1c, ITGB 1, CD29, ITGB2, IL15Ra, IL7R, CD18, CD132, LFA-1, ITGB7, KG2D, KG2C, T FR2, TRANCE/RA KL, DNAM1 (CD226), SLAMF4 (CD244, 2B4), CD84, CD96 (Tactile), CEACAM1, CRT AM, Ly9 (CD229), CD 160 (BY55), PSGL1, CD 100 (SEMA4D), CD69, SLAMF6 (NTB-A, Ly108), SLAM (SLAMF1, CD 150, IPO-3), BLAME (SLAMF8), SELPLG (CD 162), LTBR, LAT, GADS, SLP-76, PAG/Cbp, CD19a, DAP10, DAP 12, a ligand of CD83, an MHC class I molecule, a TNF receptor protein, an immunoglobulin-like protein, a cytokine receptor, an integrin, and/or a signaling lymphocytic activation molecule.

51.-52. (canceled)

53. The engineered polypeptide of claim 48, wherein at least one signaling domain is a immunoreceptor tyrosine-based activation motif (ITAM) signaling domain.

54. The engineered polypeptide of claim 48, wherein at least one signaling domain is a signaling domain from a receptor protein selected from CD3c, CD3, CD3η, FcRγ, FcRβ, CD3δ, CD3γ, CD5, CD22, CD20, CD79a, CD79b, CD278 (ICOS), FIERI, CD66d, DAP10, and DAP12.

55. (canceled)

56. The engineered polypeptide of claim 38, wherein the engineered polypeptide comprises, from amino terminus to carboxy-terminus, (i) an extracellular domain [ECD]—a transmembrane domain [TM]—a co-stimulatory domain [CoD]—a signaling domain [SigD]—a degradation domain [DD]; or (2) ECD-TM-CoD-DD-SigD; or (3) ECD-TM-DD-CoD-SigD.

57. The engineered polypeptide of claim 38, wherein the engineered polypeptide is a chimeric antigen receptor (CAR).

58.-61. (canceled)

62. An isolated nucleic acid molecule comprising a polynucleotide sequence that encodes the engineered polypeptide of claim 1.

63. A vector comprising the nucleic acid molecule of claim 62.

64. (canceled)

65. A cell comprising the engineered polypeptide of claim 1 or a nucleic acid molecule comprising a polynucleotide sequence that encodes the engineered polypeptide.

66. The cell of claim 65, wherein the cell is a human effector cell.

67. The cell of claim 65, wherein the cell is a T cell or NK cell.

68. (canceled)

69. The cell of claim 67, wherein the cell is a T effector cell.

70. The cell of claim 67, wherein the cell is a CD4+ T cell or a CD8+ T cell.

71. A pharmaceutical composition comprising the cell of claim 65.

72. A method of reducing the level of the engineered polypeptide of claim 1, comprising contacting the engineered polypeptide with a degradation agent selected from: 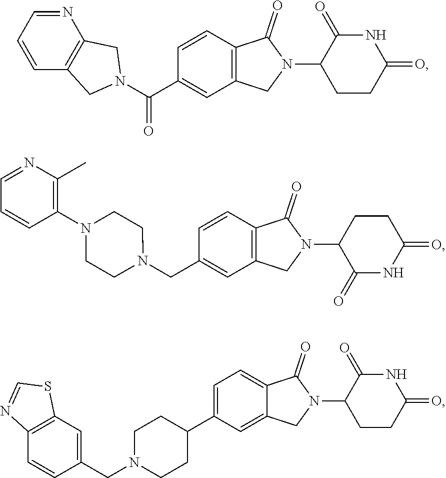 [SMILES: O=C1CCC(N2Cc3cc(C(=O)N4Cc5cccnc5C4)ccc3C2=O)C(=O)N1 ; Cc1ncccc1N1CCN(Cc2ccc3c(c2)CN(C2CCC(=O)NC2=O)C3=O)CC1 ; O=C1CCC(N2Cc3cc(C4CCN(Cc5ccc6ncsc6c5)CC4)ccc3C2=O)C(=O)N1]
and tautomers thereof, and pharmaceutically acceptable salts thereof, and pharmaceutically acceptable salts of tautomers thereof, and wherein the engineered polypeptide is comprised in a cell.

73. A method of reducing the level of an engineered polypeptide in a cell, comprising contacting the cell of claim 65 with a degradation agent selected from: 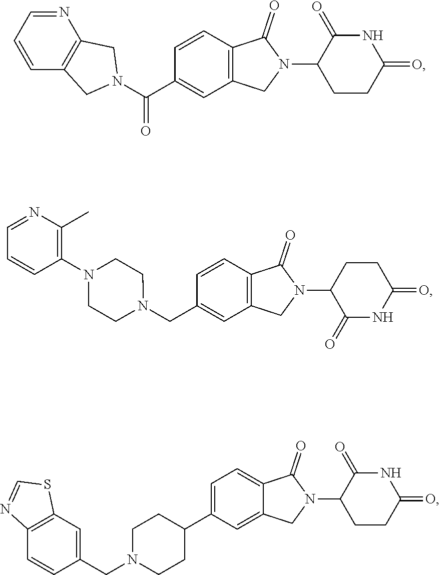 [SMILES: O=C1CCC(N2Cc3cc(C(=O)N4Cc5cccnc5C4)ccc3C2=O)C(=O)N1 ; Cc1ncccc1N1CCN(Cc2ccc3c(c2)CN(C2CCC(=O)NC2=O)C3=O)CC1 ; O=C1CCC(N2Cc3cc(C4CCN(Cc5ccc6ncsc6c5)CC4)ccc3C2=O)C(=O)N1]
and tautomers thereof, and pharmaceutically acceptable salts thereof, and pharmaceutically acceptable salts of tautomers thereof.

74. (canceled)

75. The method of claim 72, wherein the cell is a human effector cell.

76. The method of claim 72, wherein the cell is a T cell or NK cell.

77. The method of claim 72, wherein the engineered polypeptide is ubiquitinated and degraded in the presence of the degradation agent.

78.-80. (canceled)

81. The method of claim 72, wherein the degradation agent also binds to cereblon and mediates a complex comprising the degradation domain, degradation agent, and cereblon.

82. A method of treating a disease or disorder in a subject, comprising administering to the subject a cell of claim 67.

83.-84. (canceled)

85. The method of claim 82, wherein the cell is a CD4+ T cell or a CD8+ T cell.

86. The method of claim 82, wherein the engineered polypeptide is a chimeric antigen receptor (CAR).

87. The method of claim 82, wherein the disease or disorder is cancer.

88. The method of claim 87, wherein the cancer is selected from a hematological cancer or a solid cancer, and wherein:
the hematological cancer is acute myelogenous leukemia (AML), acute lymphoblastic leukemia (ALL), chronic myelogenous leukemia (CML), a lymphoma, non-Hodgkin lymphoma (NHL), Hodgkin's disease, multiple myeloma, or a myelodysplastic syndrome; or
the solid cancer is brain cancer, prostate cancer, breast cancer, lung cancer, colon cancer, uterine cancer, skin cancer, liver cancer, bone cancer, pancreatic cancer, ovarian cancer, testicular cancer, bladder cancer, kidney cancer, head and neck cancer, stomach cancer, cervical cancer, rectal cancer, larynx cancer, and esophageal cancer.

89.-90. (canceled)

91. The method of claim 82, wherein:
the cell is contacted ex vivo with a degradation agent prior to administering the cell to the subject;
the cell is administered to the subject with a degradation agent; or
a degradation agent is administered after the cell is administered to the subject; and
wherein the degradation agent is a small molecule that binds to the degradation domain.

92.-93. (canceled)

94. The method of claim 91, wherein the degradation agent is a compound selected from: 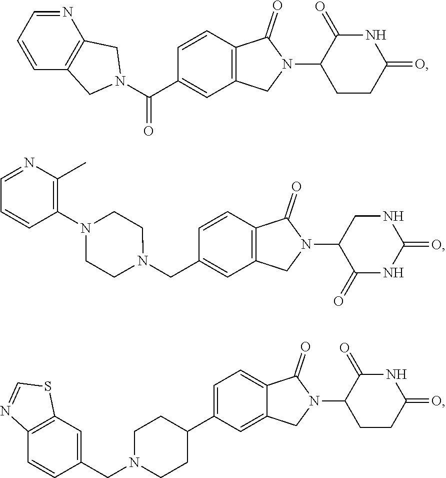 [SMILES: O=C1CCC(N2Cc3cc(C(=O)N4Cc5cccnc5C4)ccc3C2=O)C(=O)N1 ; Cc1ncccc1N1CCN(Cc2ccc3c(c2)CN(C2CNC(=O)NC2=O)C3=O)CC1 ; O=C1CCC(N2Cc3cc(C4CCN(Cc5ccc6ncsc6c5)CC4)ccc3C2=O)C(=O)N1]
and tautomers thereof, pharmaceutically acceptable salts thereof, and pharmaceutically acceptable salts of tautomers thereof.

95. A compound selected from: 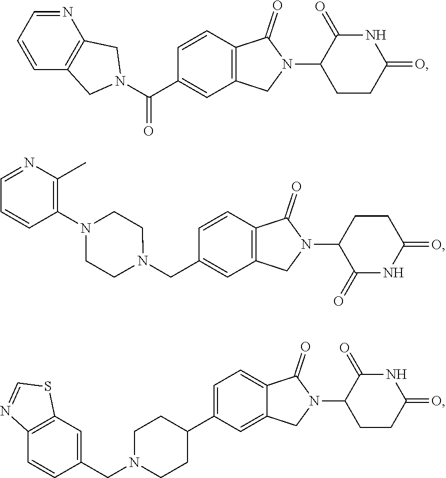 [SMILES: O=C1CCC(N2Cc3cc(C(=O)N4Cc5cccnc5C4)ccc3C2=O)C(=O)N1 ; Cc1ncccc1N1CCN(Cc2ccc3c(c2)CN(C2CCC(=O)NC2=O)C3=O)CC1 ; O=C1CCC(N2Cc3cc(C4CCN(Cc5ccc6ncsc6c5)CC4)ccc3C2=O)C(=O)N1]
and tautomers thereof, pharmaceutically acceptable salts thereof, and pharmaceutically acceptable salts of tautomers thereof.

96. A pharmaceutical composition comprising the compound of claim 95 and at least one pharmaceutically acceptable carrier.


SEQUENCE LISTING (109 sequences)

SEQ ID NO: 1 - protein, 8 aa, synthetic construct
  REGION 1..8: note = Synthetic: X1F G-motif consensus 1
  SITE 3: note = misc_feature - Xaa can be asparagine, aspartate, glycine, glutamine, methionine,histidine, tryptophan, isoleucine, arginine, leucine, valine,threonine, or phenylalanine
  SITE 4: note = misc_feature - Xaa can be glutamine, arginine, histidine, leucine,phenylalanine, tyrosine, tryptophan, isoleucine, valine, ormethionine
  SITE 7: note = misc_feature - Xaa can be alanine, serine, cysteine, arginine, leucine,isoleucine, methionine, or glycine
  SITE 8: note = misc_feature - Xaa can be serine, methionine, lysine, isoleucine, valine,histidine, glutamine, arginine, phenylalanine, or tryptophan
FCXXCGXX

SEQ ID NO: 2 - protein, 9 aa, synthetic construct
  REGION 1..9: note = Synthetic: X1F G-motif consensus 2
  SITE 3: note = misc_feature - Xaa can be asparagine, aspartate, glycine, glutamine, methionine,histidine, tryptophan, isoleucine, arginine, leucine, valine,threonine, or phenylalanine
  SITE 4: note = misc_feature - Xaa can be glutamine, arginine, histidine, leucine,phenylalanine, tyrosine, tryptophan, isoleucine, valine, ormethionine
  SITE 7: note = misc_feature - Xaa can be alanine, serine, cysteine, arginine, leucine,isoleucine, methionine, or glycine
  SITE 8: note = misc_feature - Xaa can be serine, methionine, lysine, isoleucine, valine,histidine, glutamine, arginine, phenylalanine, or tryptophan
  SITE 9: note = misc_feature - Xaa can be phenylalanine, tryptophan, methionine, arginine,histidine, leucine, tyrosine, cysteine, or glutamine
FCXXCGXXX

SEQ ID NO: 3 - protein, 8 aa, synthetic construct
  REGION 1..8: note = Synthetic: IKZF1 ZNF2 Q1F G-motif
FCNQCGAS

SEQ ID NO: 4 - protein, 28 aa, synthetic construct
  REGION 1..28: note = Synthetic: IKZF1 ZNF2 Q1F
GERPFFCNQCGASFTQKGNLLRHIKLHS

SEQ ID NO: 5 - protein, 56 aa, synthetic construct
  REGION 1..56: note = Synthetic: IKZF1 ZNF2_ZNF3 Q1F consensus
  SITE 8: note = misc_feature - Xaa can be asparagine, aspartate, glycine, glutamine, methionine,histidine, tryptophan, isoleucine, arginine, leucine, valine,threonine, or phenylalanine
  SITE 9: note = misc_feature - Xaa can be glutamine, arginine, histidine, leucine,phenylalanine, tyrosine, tryptophan, isoleucine, valine, ormethionine
  SITE 12: note = misc_feature - Xaa can be alanine, serine, cysteine, arginine, leucine,isoleucine, methionine, or glycine
  SITE 13: note = misc_feature - Xaa can be serine, methionine, lysine, isoleucine, valine,histidine, glutamine, arginine, phenylalanine, or tryptophan
  SITE 14: note = misc_feature - Xaa can be phenylalanine, tryptophan, methionine, arginine,histidine, leucine, tyrosine, cysteine, or glutamine
GERPFFCXXCGXXXTQKGNLLRHIKLHSGEKPFKCHLCNYACRRRDALTGHLRTHS

SEQ ID NO: 6 - protein, 56 aa, synthetic construct
  REGION 1..56: note = Synthetic: IKZF1 ZNF2_ZNF3
GERPFQCNQCGASFTQKGNLLRHIKLHSGEKPFKCHLCNYACRRRDALTGHLRTHS

SEQ ID NO: 7 - protein, 56 aa, synthetic construct
  REGION 1..56: note = Synthetic: IKZF1 ZNF2_ZNF3 Q1F
GERPFFCNQCGASFTQKGNLLRHIKLHSGEKPFKCHLCNYACRRRDALTGHLRTHS

SEQ ID NO: 8 - DNA, 180 bp, synthetic construct
  misc_feature 1..180: note = Synthetic: IKZF1 ZNF1_ZNF2 nucleotide sequence
  source 1..180: mol_type = other DNA
ggcattcgacttcctaacggaaaactaaagtgtgatatctgtgggatcatttgcatcgggcccaatgtgctcatggttcacaaaagaagccacactggagaacggcccttccagtgcaatcagtgcggggcctcattcacccagaagggcaacctgctccggcacatcaagctgcattcc

SEQ ID NO: 9 - protein, 60 aa, synthetic construct
  REGION 1..60: note = Synthetic: IKZF1 ZNF1_ZNF2
GIRLPNGKLKCDICGIICIGPNVLMVHKRSHTGERPFQCNQCGASFTQKGNLLRHIKLHS

SEQ ID NO: 10 - DNA, 264 bp, synthetic construct
  misc_feature 1..264: note = Synthetic: IKZF1 ZNF1_ZNF2_ ZNF3 nucleotide sequence
  source 1..264: mol_type = other DNA
ggcattcgacttcctaacggaaaactaaagtgtgatatctgtgggatcatttgcatcgggcccaatgtgctcatggttcacaaaagaagccacactggagaacggcccttccagtgcaatcagtgcggggcctcattcacccagaagggcaacctgctccggcacatcaagctgcattccggggagaagcccttcaaatgccacctctgcaactacgcctgccgccggagggacgccctcactggccacctgaggacgcactcc

SEQ ID NO: 11 - protein, 88 aa, synthetic construct
  REGION 1..88: note = Synthetic: IKZF1 ZNF1_ZNF2_ ZNF3
GIRLPNGKLKCDICGIICIGPNVLMVHKRSHTGERPFQCNQCGASFTQKGNLLRHIKLHSGEKPFKCHLCNYACRRRDALTGHLRTHS

SEQ ID NO: 12 - DNA, 168 bp, synthetic construct
  misc_feature 1..168: note = Synthetic: IKZF1 ZNF2_ZNF3 nucleotide sequence
  source 1..168: mol_type = other DNA
ggagaacggcccttccagtgcaatcagtgcggggcctcattcacccagaagggcaacctgctccggcacatcaagctgcattccggggagaagcccttcaaatgccacctctgcaactacgcctgccgccggagggacgccctcactggccacctgaggacgcactcc

SEQ ID NO: 13 - protein, 56 aa, synthetic construct; identical to SEQ ID NO: 6
  REGION 1..56: note = Synthetic: IKZF1 ZNF2_ZNF3

SEQ ID NO: 14 - DNA, 84 bp, synthetic construct
  misc_feature 1..84: note = Synthetic: IKZF1 ZNF2 nucleotide sequence
  source 1..84: mol_type = other DNA
ggagaacggcccttccagtgcaatcagtgcggggcctcattcacccagaagggcaacctgctccggcacatcaagctgcattcc

SEQ ID NO: 15 - protein, 28 aa, synthetic construct
  REGION 1..28: note = Synthetic: IKZF1 ZNF2
GERPFQCNQCGASFTQKGNLLRHIKLHS

SEQ ID NO: 16 - DNA, 168 bp, synthetic construct
  misc_feature 1..168: note = Synthetic: IKZF1 ZNF2_ZNF3 G6N nucleotide sequence
  source 1..168: mol_type = other DNA
ggagaacggcccttccagtgcaatcagtgcaacgcctcattcacccagaagggcaacctgctccggcacatcaagctgcattccggggagaagcccttcaaatgccacctctgcaactacgcctgccgccggagggacgccctcactggccacctgaggacgcactcc

SEQ ID NO: 17 - protein, 56 aa, synthetic construct
  REGION 1..56: note = Synthetic: IKZF1 ZNF2_ZNF3 G6N
GERPFQCNQCNASFTQKGNLLRHIKLHSGEKPFKCHLCNYACRRRDALTGHLRTHS

SEQ ID NO: 18 - DNA, 168 bp, synthetic construct
  misc_feature 1..168: note = Synthetic: IKZF1 ZNF2_ZNF3 Q1F nucleotide sequence
  source 1..168: mol_type = other DNA
ggagaacggcccttcttctgcaatcagtgcggggcctcattcacccagaagggcaacctgctccggcacatcaagctgcattccggggagaagcccttcaaatgccacctctgcaactacgcctgccgccggagggacgccctcactggccacctgaggacgcactcc

SEQ ID NO: 19 - protein, 56 aa, synthetic construct; identical to SEQ ID NO: 7
  REGION 1..56: note = Synthetic: IKZF1 ZNF2_ZNF3 Q1F

SEQ ID NO: 20 - protein, 23 aa, synthetic construct
  REGION 1..23: note = Synthetic: IKZF1 ZNF1 (amino acids 117-139)
LKCDICGIICIGPNVLMVHKRSH

SEQ ID NO: 21 - protein, 23 aa, synthetic construct
  REGION 1..23: note = Synthetic: IKZF1 ZNF2 (amino acids 145-167)
FQCNQCGASFTQKGNLLRHIKLH

SEQ ID NO: 22 - protein, 23 aa, synthetic construct
  REGION 1..23: note = Synthetic: IKZF1 ZNF3 (amino acids 173-195)
FKCHLCNYACRRRDALTGHLRTH

SEQ ID NO: 23 - protein, 24 aa, synthetic construct
  REGION 1..24: note = Synthetic: IKZF1 ZNF4 (amino acids 201-224)
HKCGYCGRSYKQRSSLEEHKERCH

SEQ ID NO: 24 - protein, 23 aa, synthetic construct
  REGION 1..23: note = Synthetic: IKZF1 ZNF5 (amino acids 462-484)
YKCEHCRVLFLDHVMYTIHMGCH

SEQ ID NO: 25 - protein, 25 aa, synthetic construct
  REGION 1..25: note = Synthetic: IKZF1 ZNF6 (amino acids 490-514)
FECNMCGYHSQDRYEFSSHITRGEH

SEQ ID NO: 26 - protein, 23 aa, synthetic construct
  REGION 1..23: note = Synthetic: IKZF2 ZNF1 (amino acids 112-134)
LKCDVCGMVCIGPNVLMVHKRSH

SEQ ID NO: 27 - protein, 23 aa, synthetic construct
  REGION 1..23: note = Synthetic: IKZF2 ZNF2 (amino acids 140-162)
FHCNQCGASFTQKGNLLRHIKLH

SEQ ID NO: 28 - protein, 23 aa, synthetic construct
  REGION 1..23: note = Synthetic: IKZF2 ZNF3 (amino acids 168-190)
FKCPFCSYACRRRDALTGHLRTH

SEQ ID NO: 29 - protein, 24 aa, synthetic construct
  REGION 1..24: note = Synthetic: IKZF2 ZNF4 (amino acids 196-219)
HKCNYCGRSYKQRSSLEEHKERCH

SEQ ID NO: 30 - protein, 23 aa, synthetic construct
  REGION 1..23: note = Synthetic: IKZF2 ZNF5 (amino acids 471-493)
FKCEHCRVLFLDHVMYTIHMGCH

SEQ ID NO: 31 - protein, 23 aa, synthetic construct
  REGION 1..23: note = Synthetic: IKZF3 ZNF1 (amino acids 118-140)
MNCDVCGLSCISFNVLMVHKRSH

SEQ ID NO: 32 - protein, 23 aa, synthetic construct
  REGION 1..23: note = Synthetic: IKZF3 ZNF2 (amino acids 146-168)
FQCNQCGASFTQKGNLLRHIKLH

SEQ ID NO: 33 - protein, 23 aa, synthetic construct
  REGION 1..23: note = Synthetic: IKZF3 ZNF3 (amino acids 174-196)
FKCHLCNYACQRRDALTGHLRTH

SEQ ID NO: 34 - protein, 23 aa, synthetic construct
  REGION 1..23: note = Synthetic: IKZF3 ZNF4 (amino acids 202-224)
YKCEFCGRSYKQRSSLEEHKERC

SEQ ID NO: 35 - protein, 23 aa, synthetic construct
  REGION 1..23: note = Synthetic: IKZF3 ZNF5 (amino acids 452-474)
YRCDHCRVLFLDYVMFTIHMGCH

SEQ ID NO: 36 - protein, 25 aa, synthetic construct
  REGION 1..25: note = Synthetic: IKZF3 ZNF6 (amino acids 480-504)
FECNMCGYRSHDRYEFSSHIARGEH

SEQ ID NO: 37 - protein, 23 aa, synthetic construct
  REGION 1..23: note = Synthetic: IKZF4 ZNF1 (amino acids 159-181)
LKCDVCGMVCIGPNVLMVHKRSH

SEQ ID NO: 38 - protein, 23 aa, synthetic construct
  REGION 1..23: note = Synthetic: IKZF4 ZNF2 (amino acids 187-209)
FHCNQCGASFTQKGNLLRHIKLH

SEQ ID NO: 39 - protein, 23 aa, synthetic construct
  REGION 1..23: note = Synthetic: IKZF4 ZNF3 (amino acids 215-237)
FKCPFCNYACRRRDALTGHLRTH

SEQ ID NO: 40 - protein, 24 aa, synthetic construct
  REGION 1..24: note = Synthetic: IKZF4 ZNF4 (amino acids 248-271)
YKCNYCGRSYKQQSTLEEHKERCH

SEQ ID NO: 41 - protein, 23 aa, synthetic construct
  REGION 1..23: note = Synthetic: IKZF4 ZNF5 (amino acids 530-552)
FKCEHCRILFLDHVMFTIHMGCH

SEQ ID NO: 42 - protein, 25 aa, synthetic construct
  REGION 1..25: note = Synthetic: IKZF4 ZNF6 (amino acids 558-582)
FECNICGYHSQDRYEFSSHIVRGEH

SEQ ID NO: 43 - protein, 23 aa, synthetic construct
  REGION 1..23: note = Synthetic: IKZF5 ZNF1 (amino acids 82-104)
LKCRYCNYASKGTARLIEHIRIH

SEQ ID NO: 44 - protein, 23 aa, synthetic construct
  REGION 1..23: note = Synthetic: IKZF5 ZNF2 (amino acids 110-132)
HRCHLCPFASAYERHLEAHMRSH

SEQ ID NO: 45 - protein, 24 aa, synthetic construct
  REGION 1..24: note = Synthetic: IKZF5 ZNF3 (amino acids 138-161)
YKCELCSFRCSDRSNLSHHRRRKH

SEQ ID NO: 46 - protein, 23 aa, synthetic construct
  REGION 1..23: note = Synthetic: IKZF5 ZNF4 (amino acids 364-386)
HHCQHCDMYFADNILYTIHMGCH

SEQ ID NO: 47 - protein, 25 aa, synthetic construct
  REGION 1..25: note = Synthetic: IKZF5 ZNF5 (amino acids 392-416)
FQCNICGCKCKNKYDFACHFARGQH

SEQ ID NO: 48 - protein, 519 aa, Homo sapiens
  REGION 1..519: note = misc_feature - Human Ikaros amino acid sequence
MDADEGQDMSQVSGKESPPVSDTPDEGDEPMPIPEDLSTTSGGQQSSKSDRVVASNVKVETQSDEENGRACEMNGEECAEDLRMLDASGEKMNGSHRDQGSSALSGVGGIRLPNGKLKCDICGIICIGPNVLMVHKRSHTGERPFQCNQCGASFTQKGNLLRHIKLHSGEKPFKCHLCNYACRRRDALTGHLRTHSVGKPHKCGYCGRSYKQRSSLEEHKERCHNYLESMGLPGTLYPVIKEETNHSEMAEDLCKIGSERSLVLDRLASNVAKRKSSMPQKFLGDKGLSDTPYDSSASYEKENEMMKSHVMDQAINNAINYLGAESLRPLVQTPPGGSEVVPVISPMYQLHKPLAEGTPRSNHSAQDSAVENLLLLSKAKLVPSEREASPSNSCQDSTDTESNNEEQRSGLIYLTNHIAPHARNGLSLKEEHRAYDLLRAASENSQDALRVVSTSGEQMKVYKCEHCRVLFLDHVMYTIHMGCHGFRDPFECNMCGYHSQDRYEFSSHITRGEHRFHMS

SEQ ID NO: 49 - protein, 509 aa, Homo sapiens
  REGION 1..509: note = misc_feature - Human Aiolos amino acid sequence
MEDIQTNAELKSTQEQSVPAESAAVLNDYSLTKSHEMENVDSGEGPANEDEDIGDDSMKVKDEYSERDENVLKSEPMGNAEEPEIPYSYSREYNEYENIKLERHVVSFDSSRPTSGKMNCDVCGLSCISFNVLMVHKRSHTGERPFQCNQCGASFTQKGNLLRHIKLHTGEKPFKCHLCNYACQRRDALTGHLRTHSVEKPYKCEFCGRSYKQRSSLEEHKERCRTFLQSTDPGDTASAEARHIKAEMGSERALVLDRLASNVAKRKSSMPQKFIGEKRHCFDVNYNSSYMYEKESELIQTRMMDQAINNAISYLGAEALRPLVQTPPAPTSEMVPVISSMYPIALTRAEMSNGAPQELEKKSIHLPEKSVPSERGLSPNNSGHDSTDTDSNHEERQNHIYQQNHMVLSRARNGMPLLKEVPRSYELLKPPPICPRDSVKVINKEGEVMDVYRCDHCRVLFLDYVMFTIHMGCHGFRDPFECNMCGYRSHDRYEFSSHIARGEHRALLK

SEQ ID NO: 50 - protein, 526 aa, Homo sapiens [sequence omitted]

SEQ ID NO: 51 - protein, 585 aa, Homo sapiens
  REGION 1..585: note = misc_feature - Human Eos amino acid sequence
MHTPPALPRRFQGGGRVRTPGSHRQGKDNLERDPSGGCVPDFLPQAQDSNHFIMESLFCESSGDSSLEKEFLGAPVGPSVSTPNSQHSSPSRSLSANSIKVEMYSDEESSRLLGPDERLLEKDDSVIVEDSLSEPLGYCDGSGPEPHSPGGIRLPNGKLKCDVCGMVCIGPNVLMVHKRSHTGERPFHCNQCGASFTQKGNLLRHIKLHSGEKPFKCPFCNYACRRRDALTGHLRTHSVSSPTVGKPYKCNYCGRSYKQQSTLEEHKERCHNYLQSLSTEAQALAGQPGDEIRDLEMVPDSMLHSSSERPTFIDRLANSLTKRKRSTPQKFVGEKQMRFSLSDLPYDVNSGGYEKDVELVAHHSLEPGFGSSLAFVGAEHLRPLRLPPTNCISELTPVISSVYTQMQPLPGRLELPGSREAGEGPEDLADGGPLLYRPRGPLTDPGASPSNGCQDSTDTESNHEDRVAGVVSLPQGPPPQPPPTIVVGRHSPAYAKEDPKPQEGLLRGTPGPSKEVLRVVGESGEPVKAFKCEHCRILFLDHVMFTIHMGCHGFRDPFECNICGYHSQDRYEFSSHIVRGEHKVG

SEQ ID NO: 52 - protein, 419 aa, Homo sapiens
  REGION 1..419: note = misc_feature - Human Pegasus amino acid sequence
MGEKKPEPLDFVKDFQEYLTQQTHHVNMISGSVSGDKEAEALQGAGTDGDQNGLDHPSVEVSLDENSGMLVDGFERTFDGKLKCRYCNYASKGTARLIEHIRIHTGEKPHRCHLCPFASAYERHLEAHMRSHTGEKPYKCELCSFRCSDRSNLSHHRRRKHKMVPIKGTRSSLSSKKMWGVLQKKTSNLGYSRRALINLSPPSMVVQKPDYLNDFTHEIPNIQTDSYESMAKTTPTGGLPRDPQELMVDNPLNQLSTLAGQLSSLPPENQNPASPDVVPCPDEKPFMIQQPSTQAVVSAVSASIPQSSSPTSPEPRPSHSQRNYSPVAGPSSEPSAHTSTPSIGNSQPSTPAPALPVQDPQLLHHCQHCDMYFADNILYTIHMGCHGYENPFQCNICGCKCKNKYDFACHFARGQHNQH

SEQ ID NO: 53 - protein, 459 aa, synthetic construct
  REGION 1..459: note = Synthetic: CAR CD19 CD28TM 41BB CD3z
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGGGEGRGSLLTCGDVEENPG

SEQ ID NO: 54 - protein, 488 aa, synthetic construct
  REGION 1..488: note = Synthetic: CD28TM 41BB CD3z ZNF2
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTGERPFQCNQCGASFTQKGNLLRHIKLHSGGGEGRGSLLTCGDVEENPG

SEQ ID NO: 55 - protein, 519 aa, synthetic construct
  REGION 1..519: note = Synthetic: CD28TM 41BB CD3z ZNF1_2
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGIRLPNGKLKCDICGIICIGPNVLMVHKRSHTGERPFQCNQCGASFTQKGNLLRHIKLHSGGGEGRGSLLTCGDVEENPG

SEQ ID NO: 56 - protein, 515 aa, synthetic construct
  REGION 1..515: note = Synthetic: CD28TM 41BB CD3z ZNF2_3
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGERPFQCNQCGASFTQKGNLLRHIKLHSGEKPFKCHLCNYACRRRDALTGHLRTHSGGGEGRGSLLTCGDVEENPG

SEQ ID NO: 57 - protein, 547 aa, synthetic construct
  REGION 1..547: note = Synthetic: CD28TM 41BB CD3z ZNF1_2_3
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGIRLPNGKLKCDICGIICIGPNVLMVHKRSHTGERPFQCNQCGASFTQKGNLLRHIKLHSGEKPFKCHLCNYACRRRDALTGHLRTHSGGGEGRGSLLTCGDVEENPG

SEQ ID NO: 58 - protein, 25 aa, synthetic construct
  REGION 1..25: note = Synthetic: IKZF2 ZNF6 (amino acids 499-523)
LECNICGYRSQDRYEFSSHIVRGEH

SEQ ID NO: 59 - protein, 56 aa, synthetic construct
  REGION 1..56: note = Synthetic: IKZF1 ZNF2_ZNF3 Q1F/G6N
GERPFFCNQCNASFTQKGNLLRHIKLHSGEKPFKCHLCNYACRRRDALTGHLRTHS

SEQ ID NO: 60 - protein, 28 aa, synthetic construct
  REGION 1..28: note = Synthetic: IKZF2 ZNF2
GERPFHCNQCGASFTQKGNLLRHIKLHS

SEQ ID NO: 61 - protein, 28 aa, synthetic construct
  REGION 1..28: note = Synthetic: IKZF3 ZNF2
GERPFQCNQCGASFTQKGNLLRHIKLHT

SEQ ID NO: 62 - protein, 28 aa, synthetic construct
  REGION 1..28: note = Synthetic: IKZF4 ZNF2
GERPFHCNQCGASFTQKGNLLRHIKLHS

SEQ ID NO: 63 - protein, 56 aa, synthetic construct
  REGION 1..56: note = Synthetic: IKZF2 ZNF2_ZNF3
GERPFHCNQCGASFTQKGNLLRHIKLHSGEKPFKCPFCSYACRRRDALTGHLRTHS

SEQ ID NO: 64 - protein, 56 aa, synthetic construct
  REGION 1..56: note = Synthetic: IKZF3 ZNF2_ZNF3
GERPFQCNQCGASFTQKGNLLRHIKLHTGEKPFKCHLCNYACQRRDALTGHLRTHS

SEQ ID NO: 65 - protein, 56 aa, synthetic construct
  REGION 1..56: note = Synthetic: IKZF4 ZNF2_ZNF3
GERPFHCNQCGASFTQKGNLLRHIKLHSGEKPFKCPFCNYACRRRDALTGHLRTHS

SEQ ID NO: 66 - protein, 56 aa, synthetic construct
  REGION 1..56: note = Synthetic: IKZF2 ZNF2_ZNF3 Q1F consensus
  SITE 8: note = misc_feature - Xaa can be asparagine, aspartate, glycine, glutamine, methionine,histidine, tryptophan, isoleucine, arginine, leucine, valine,threonine, or phenylalanine
  SITE 9: note = misc_feature - Xaa can be glutamine, arginine, histidine, leucine,phenylalanine, tyrosine, tryptophan, isoleucine, valine, ormethionine
  SITE 12: note = misc_feature - Xaa can be alanine, serine, cysteine, arginine, leucine,isoleucine, methionine, or glycine
  SITE 13: note = misc_feature - Xaa can be serine, methionine, lysine, isoleucine, valine,histidine, glutamine, arginine, phenylalanine, or tryptophan
  SITE 14: note = misc_feature - Xaa can be phenylalanine, tryptophan, methionine, arginine,histidine, leucine, tyrosine, cysteine, or glutamine
GERPFFCXXCGXXXTQKGNLLRHIKLHSGEKPFKCPFCSYACRRRDALTGHLRTHS

SEQ ID NO: 67 - protein, 56 aa, synthetic construct
  REGION 1..56: note = Synthetic: IKZF3 ZNF2_ZNF3 Q1F consensus
  SITE 8: note = misc_feature - Xaa can be asparagine, aspartate, glycine, glutamine, methionine,histidine, tryptophan, isoleucine, arginine, leucine, valine,threonine, or phenylalanine
  SITE 9: note = misc_feature - Xaa can be glutamine, arginine, histidine, leucine,phenylalanine, tyrosine, tryptophan, isoleucine, valine, ormethionine
  SITE 12: note = misc_feature - Xaa can be alanine, serine, cysteine, arginine, leucine,isoleucine, methionine, or glycine
  SITE 13: note = misc_feature - Xaa can be serine, methionine, lysine, isoleucine, valine,histidine, glutamine, arginine, phenylalanine, or tryptophan
  SITE 14: note = misc_feature - Xaa can be phenylalanine, tryptophan, methionine, arginine,histidine, leucine, tyrosine, cysteine, or glutamine
GERPFFCXXCGXXXTQKGNLLRHIKLHTGEKPFKCHLCNYACQRRDALTGHLRTHS

SEQ ID NO: 68 - protein, 56 aa, synthetic construct
  REGION 1..56: note = Synthetic: IKZF4 ZNF2_ZNF3 Q1F consensus
  SITE 8: note = misc_feature - Xaa can be asparagine, aspartate, glycine, glutamine, methionine,histidine, tryptophan, isoleucine, arginine, leucine, valine,threonine, or phenylalanine
  SITE 9: note = misc_feature - Xaa can be glutamine, arginine, histidine, leucine,phenylalanine, tyrosine, tryptophan, isoleucine, valine, ormethionine
  SITE 12: note = misc_feature - Xaa can be alanine, serine, cysteine, arginine, leucine,isoleucine, methionine, or glycine
  SITE 13: note = misc_feature - Xaa can be serine, methionine, lysine, isoleucine, valine,histidine, glutamine, arginine, phenylalanine, or tryptophan
  SITE 14: note = misc_feature - Xaa can be phenylalanine, tryptophan, methionine, arginine,histidine, leucine, tyrosine, cysteine, or glutamine
GERPFFCXXCGXXXTQKGNLLRHIKLHSGEKPFKCPFCNYACRRRDALTGHLRTHS

SEQ ID NO: 69 - protein, 56 aa, synthetic construct
  REGION 1..56: note = Synthetic: IKZF2 ZNF2_ZNF3 Q1F
GERPFFCNQCGASFTQKGNLLRHIKLHSGEKPFKCPFCSYACRRRDALTGHLRTHS

SEQ ID NO: 70 - protein, 56 aa, synthetic construct
  REGION 1..56: note = Synthetic: IKZF3 ZNF2_ZNF3 Q1F
GERPFFCNQCGASFTQKGNLLRHIKLHTGEKPFKCHLCNYACQRRDALTGHLRTHS

SEQ ID NO: 71 - protein, 56 aa, synthetic construct
  REGION 1..56: note = Synthetic: IKZF4 ZNF2_ZNF3 Q1F
GERPFFCNQCGASFTQKGNLLRHIKLHSGEKPFKCPFCNYACRRRDALTGHLRTHS

SEQ ID NO: 72 - protein, 23 aa, Homo sapiens
  REGION 1..23: note = misc_feature - Q9UKT9_IKZF3_146_168_23|CHAIN
FQCNQCGASFTQKGNLLRHIKLH

SEQ ID NO: 73 - protein, 23 aa, Homo sapiens
  REGION 1..23: note = misc_feature - Q66K89_E4F1_220_242_23|CHAIN
HECKLCGASFRTKGSLIRHHRRH

SEQ ID NO: 74 - protein, 23 aa, Homo sapiens
  REGION 1..23: note = misc_feature - Q6ZMY9_ZN517_205_227_23|CHAIN
FQCTECGKAFKQSSILLRHQLIH

SEQ ID NO: 75 - protein, 23 aa, Homo sapiens
  REGION 1..23: note = misc_feature - Q6DD87_ZN787_178_200_23|CHAIN
FVCPRCGRGFSQPKSLARHLRLH

SEQ ID NO: 76 - protein, 23 aa, Homo sapiens
  REGION 1..23: note = misc_feature - Q6ZMY9_ZN517_396_418_23|CHAIN
FECAECGKAFGRKSNLTLHQKIH

SEQ ID NO: 77 - protein, 23 aa, Homo sapiens
  REGION 1..23: note = misc_feature - Q96JP5_ZFP91_400_422_23|CHAIN
LQCEICGFTCRQKASLNWHMKKH

SEQ ID NO: 78 - protein, 23 aa, Homo sapiens
  REGION 1..23: note = misc_feature - Q6DD87_ZN787_150_172_23|CHAIN
YTCPDCGRSFTQSKSLAKHRRSH

SEQ ID NO: 79 - protein, 23 aa, Homo sapiens
  REGION 1..23: note = misc_feature - Q96CK0_ZN653_528_550_23|CHAIN
FTCETCGKSFKRKNHLEVHRRTH

SEQ ID NO: 80 - protein, 23 aa, Homo sapiens
  REGION 1..23: note = misc_feature - Q17R98_ZN827_817_839_23|CHAIN
FPCDVCGKVFGRQQTLSRHLSLH

SEQ ID NO: 81 - protein, 23 aa, Homo sapiens
  REGION 1..23: note = misc_feature - Q6DD87_ZN787_317_339_23|CHAIN
HICVECGEGFVQGAALRRHKKIH

SEQ ID NO: 82 - protein, 23 aa, Homo sapiens
  REGION 1..23: note = misc_feature - Q6DD87_ZN787_66_88_23|CHAIN
YICNECGKSFSHWSKLTRHQRTH

SEQ ID NO: 83 - protein, 23 aa, Homo sapiens
  REGION 1..23: note = misc_feature - Q6ZMY9_ZN517_233_255_23|CHAIN
FQCGECGKAFRQSTQLAAHHRVH

SEQ ID NO: 84 - protein, 23 aa, Homo sapiens
  REGION 1..23: note = misc_feature - Q96CK0_ZN653_556_578_23|CHAIN
LQCEICGYQCRQRASLNWHMKKH

SEQ ID NO: 85 - protein, 23 aa, Homo sapiens
  REGION 1..23: note = misc_feature - Q9BU19_ZN692_417_439_23|CHAIN
LQCEICGFTCRQKASLNWHQRKH

SEQ ID NO: 86 - protein, 23 aa, Homo sapiens
  REGION 1..23: note = misc_feature - Q6ZMY9_ZN517_317_339_23|CHAIN
YRCLRCGQRFIRGSSLLKHHRLH

SEQ ID NO: 87 - protein, 23 aa, Homo sapiens
  REGION 1..23: note = misc_feature - Q6ZMY9_ZN517_261_283_23|CHAIN
YACGECGKAFSRSSRLLQHQKFH

SEQ ID NO: 88 - protein, 23 aa, Homo sapiens
  REGION 1..23: note = misc_feature - Q8IZM8_ZN654_25_47_23|CHAIN
FACVICGRKFRNRGLMQKHLKNH

SEQ ID NO: 89 - protein, 23 aa, Homo sapiens
  REGION 1..23: note = misc_feature - Q17R98_ZN827_374_396_23|CHAIN
FQCPICGLVIKRKSYWKRHMVIH

SEQ ID NO: 90 - protein, 23 aa, Homo sapiens
  REGION 1..23: note = misc_feature - Q6ZMY9_ZN517_368_390_23|CHAIN
HECPVCGRPFRHNSLLLLHLRLH

SEQ ID NO: 91 - protein, 23 aa, Homo sapiens
  REGION 1..23: note = misc_feature - Q8N554_ZN276_496_518_23|CHAIN
YICDECGQTFKQRKHLLVHQMRH

SEQ ID NO: 92 - protein, 23 aa, Homo sapiens
  REGION 1..23: note = misc_feature - Q66K89_E4F1_463_485_23|CHAIN
FACAQCGKAFPKAYLLKKHQEVH

SEQ ID NO: 93 - protein, 23 aa, Homo sapiens
  REGION 1..23: note = misc_feature - Q6ZMY9_ZN517_424_446_23|CHAIN
FACTECGKAFRRSYTLNEHYRLH

SEQ ID NO: 94 - protein, 23 aa, Homo sapiens
  REGION 1..23: note = misc_feature - Q96NG8_ZN582_339_361_23|CHAIN
YECKECGKAFNQGSTLIRHQRIH

SEQ ID NO: 95 - protein, 23 aa, Homo sapiens
  REGION 1..23: note = misc_feature - Q8N554_ZN276_524_546_23|CHAIN
LQCEVCGFQCRQRASLKYHMTKH

SEQ ID NO: 96 - protein, 23 aa, Homo sapiens
  REGION 1..23: note = misc_feature - Q6DD87_ZN787_94_116_23|CHAIN
NACADCGKTFSQSSHLVQHRRIH

SEQ ID NO: 97 - protein, 23 aa, Homo sapiens
  REGION 1..23: note = misc_feature - Q6DD87_ZN787_122_144_23|CHAIN
YACLECGKRFSWSSNLMQHQRIH

SEQ ID NO: 98 - protein, 23 aa, Homo sapiens
  REGION 1..23: note = misc_feature - Q6ZMY9_ZN517_289_311_23|CHAIN
FACTECGKAFCRRFTLNEHGRIH

SEQ ID NO: 99 - protein, 23 aa, Homo sapiens
  REGION 1..23: note = misc_feature - Q96NG8_ZN582_283_305_23|CHAIN
YQCKECGKAFNRISHLKVHYRIH

SEQ ID NO: 100 - protein, 23 aa, Homo sapiens
  REGION 1..23: note = misc_feature - Q96NG8_ZN582_395_417_23|CHAIN
YQCKVCGRAFKRVSHLTVHYRIH

SEQ ID NO: 101 - protein, 23 aa, Homo sapiens
  REGION 1..23: note = misc_feature - Q6ZMY9_ZN517_452_474_23|CHAIN
YRCRACGRACSRLSTLIQHQKVH

SEQ ID NO: 102 - protein, 23 aa, Homo sapiens
  REGION 1..23: note = misc_feature - Q9UKT9_IKZF3_118_140_23|CHAIN
MNCDVCGLSCISFNVLMVHKRSH

SEQ ID NO: 103 - protein, 23 aa, Homo sapiens
  REGION 1..23: note = misc_feature - Q96NG8_ZN582_367_389_23|CHAIN
YECKVCGKAFRVSSQLKQHQRIH

SEQ ID NO: 104 - protein, 23 aa, Homo sapiens
  REGION 1..23: note = misc_feature - Q96NG8_ZN582_311_333_23|CHAIN
YACKECGKTFSHRSQLIQHQTVH

SEQ ID NO: 105 - protein, 23 aa, Homo sapiens
  REGION 1..23: note = misc_feature - Q96NG8_ZN582_423_445_23|CHAIN
YECKECGKAFSHCSQLIHHQVIH

SEQ ID NO: 106 - protein, 23 aa, Homo sapiens
  REGION 1..23: note = misc_feature - Q96NG8_ZN582_199_221_23|CHAIN
YKCKECGKAFKYGSRLIQHENIH

SEQ ID NO: 107 - protein, 23 aa, Homo sapiens
  REGION 1..23: note = misc_feature - Q96NG8_ZN582_227_249_23|CHAIN
YECKECGKAFNSGSNFIQHQRVH

SEQ ID NO: 108 - protein, 23 aa, Homo sapiens
  REGION 1..23: note = misc_feature - Q66K89_E4F1_491_513_23|CHAIN
FRCGDCGKLYKTIAHVRGHRRVH

SEQ ID NO: 109 - protein, 23 aa, Homo sapiens
  REGION 1..23: note = misc_feature - Q66K89_E4F1_519_541_23|CHAIN
YPCPKCGKRYKTKNAQQVHFRTH